SEQUENCE LISTING (183 sequences)

SEQ ID NO: 1 - skipped

SEQ ID NO: 2 - protein, 5 aa, synthetic construct
  VARIANT 3: note = X represents E or Q.
  REGION 1..5: note = each one of the amino acids being L- or D-amino acid.
SFXVR

SEQ ID NO: 3 - DNA, 21 bp, synthetic construct
  source 1..21: mol_type = other DNA
gccattggtattggggcttac

SEQ ID NO: 4 - protein, 10 aa, synthetic construct
  REGION 1..10: note = each one of the amino acids being L- or D-amino acid.
GDTNPKDDWF

SEQ ID NO: 5 - protein, 10 aa, synthetic construct
  REGION 1..10: note = each one of the amino acids being L- or D-amino acid.
  VARIANT 1: note = X represents T, or A and can be deleted. It can be represented as [X1]a, wherein X1 represnts T or A and a is 0 or 1.
  VARIANT 2: note = X represents W, N, Q, K or A and can be deleted. It can be represented as [X2]b, wherein X2 represents W, N, Q, K or A and b is 0 or 1.
  VARIANT 4: note = X represents P, N or L.
  VARIANT 7: note = 7; note = X represents A, V, L or I and it can be deleted. It can be represented as [X6]d, wherein X6 represents A, V, L or I and d is 0 or 1.
  VARIANT 8: note = X represents A, V, L or I and it can be deleted. It can be represented as [X7]e, wherein X7 represents A, V, L or I and e is 0 or 1.
  VARIANT 9: note = X represents F or P.
  VARIANT 10: note = It can be deleted. It can be represented as [Y]g, wherein g is 0 or 1.
XXDXEGXXXY

SEQ ID NO: 6 - protein, 10 aa, synthetic construct
  REGION 1..10: note = each one of the amino acids being L- or D-amino acid.
  VARIANT 1: note = X represents T, or A and it can be deleted. It can be represented as [X1]a, wherein X1 represents T or A, and a represents 0 or 1.
  VARIANT 2: note = X represents W, N, Q, K or A and it can be deleted. It can be represented as [X2]b, wherein X2 represents W, N, Q, K or A and b is 0 or 1.
  VARIANT 7: note = X represents A, V, L or I and it can be deleted. It can be represented as [X6]d, wherein X6 represents A, V, L or I and d is 0 or 1.
  VARIANT 8: note = X represents A, V, L or I, and it can be deleted. It can be represented as [X7]e, wherein X7 represents A, V, L or I, and e is 0 or 1.
  VARIANT 9: note = X represents F or P and can be deleted. It can be represented as [X8]f, wherein X8 represents For P and f is 0 or 1.
  VARIANT 10: note = It can be deleted. It can be represented as [Y]g wherein g is 0 or 1.
XXDPEGXXXY

SEQ ID NO: 7 - protein, 10 aa, synthetic construct
  REGION 1..10: note = each one of the amino acids being L- or D-amino acid.
  VARIANT 1: note = X represents T or A and it can be deleted. It can be represented as [X1]a, wherein X1 is T or A and a is 0 or 1.
  VARIANT 2: note = X represents W, N, Q, K or A and it can be deleted. It can be represented as [X2]b wherein X2 represents W, N, Q, K or A and b is 0 or 1.
  VARIANT 7: note = X represents A, V, L or I and it can be deleted. It can be represented as [X6]d wherein X6 represents A, V, L or I and d is 0 or 1.
  VARIANT 8: note = X represents A, V, L or I and it can be deleted. It can be represented as[X7]d wherein X7 represents A, V, L or I and e is 0 or 1.
  VARIANT 9: note = X represents F or P and it can be deleted. It can be represented as [X8]f wherein X8 represents F or P and f is 0 or 1.
  VARIANT 10: note = It can be deleted. It can be represented as [Y]g wherein g is 0 or 1.
XXDLEGXXXY

SEQ ID NO: 8 - protein, 10 aa, synthetic construct
  REGION 1..10: note = Each amino acid being an L-amino acid or a D-amino acid
  VARIANT 1: note = X represents T or A and it can be deleted. It can be represented as [X1]a wherein X1 represents T or A and a is 0 or 1.
  VARIANT 2: note = X represents W, N, Q, K or A and it can be deleted. It can be represented as [X2]b wherein X2 represents W, N, Q, K or A and b is 0 or 1.
  VARIANT 7: note = X represents A, V, L or I and it can be deleted. It can be represented as [X6]d wherein X6 represents A, V, L or I and d is 0 or 1.
  VARIANT 8: note = X represents A, V, L or I and it can be deleted. It can be represented as [X7]e wherein X7 represents A, V, L or I and d is 0 or 1.
  VARIANT 9: note = X represents F or P and it can be deleted. It can be represented as [X8]f wherein X8 is F or P and f is 0 or 1.
  VARIANT 10: note = It can be deleted. It can be represented as [Y]g wherein g is 0 or 1.
XXDNEGXXXY

SEQ ID NO: 9 - protein, 10 aa, synthetic construct
  REGION 1..10: note = Each amino acid being L- or a D-amino acid.
TWDPEGVIFY

SEQ ID NO: 10 - protein, 10 aa, synthetic construct
  REGION 1..10: note = Each amino acid being L- or D-amino acid
  VARIANT 1: note = It can be deleted. It can be represented as [T]a, wherein a is 0 or 1.
  VARIANT 2: note = X represents W, N, Q or K and it can be deleted. It can be represented as [X2]b, wherein X2 represents W, N, Q or K and b is 0 or 1.
  VARIANT 4: note = X represents P, N, a basic amino acid or a hydrophobic amino acid and it can be deleted. It can be represented as [X4]c wherein X4 represents P, N, a basic amino acid or a hydrophobic amino acid and c is 0 or 1.
  VARIANT 7: note = X represents A, V, L or I and it can be deleted. It can be represented as [X6]d wherein X6 represents A, V, L or I and d is 0 or 1.
  VARIANT 8: note = X represents A, V, L or I and it can be deleted. It can be represented as [X7]e wherein X7 represents A, V, L or I and e is 0 or 1.
  VARIANT 9: note = X represents F or P and it can be deleted. It can be represented as [X8]f wherein X8 represents F or P and f is 0 or 1.
  VARIANT 10: note = It can be deleted. It can be represented as [Y]g wherein g is 0 or 1.
TXDXEGXXXY

SEQ ID NO: 11 - protein, 10 aa, synthetic construct
  REGION 1..10: note = each aminoacid being an L- or D-amino acid.
  VARIANT 1: note = It can be deleted. It can be represented as [T]a wherein a is 0 por 1.
  VARIANT 2: note = X represents W, N, Q or K and it can be deleted. It can be represented as [X2]b wherein X2 represents W, N, Q or K and b is 0 or 1.
  VARIANT 4: note = X represents P, N or a basic amino acid.
  VARIANT 7: note = X represents A, V, L or I and it can be deleted. It can be represented as [X6]d wherein X6 represents A, V, L or I and d is 0 or 1.
  VARIANT 8: note = X represents A, V, L or I and it can be deleted. It can be represented as [X7]e wherein X7 represents A, V, L or I and e is 0 or 1.
  VARIANT 9: note = It can be deleted. It can be represented as [F]f whereinf is 0 or 1.
  VARIANT 10: note = It can be deleted. It can be represented as [Y]g wherein g is 0 or 1.
TXDXEGXXFY

SEQ ID NO: 12 - protein, 5 aa, synthetic construct
  REGION 1..5: note = Each amino acid being L- or D-amino acid
SFEVR

SEQ ID NO: 13 - protein, 10 aa, synthetic construct
  REGION 1..10: note = Each amino acid being L- or D-amino acid.
TKTSSSFEVR

SEQ ID NO: 14 - protein, 19 aa, synthetic construct
  REGION 1..19: note = Each amino acid being L- or D- amino acid.
VMTFDLTKITKTSSSFEVR

SEQ ID NO: 15 - protein, 20 aa, synthetic construct
  REGION 1..20: note = Each amino acid being L- or D-amino acid
GDTNPKDDWFMLGLRDGRPE

SEQ ID NO: 16 - protein, 20 aa, synthetic construct
  SITE 1..20: note = all D-amino acids
TKTSSSFEVRTWDPEGVIFY

SEQ ID NO: 17 - protein, 20 aa, synthetic construct
  SITE 1..20: note = all D-amino acids
TWDPEGVIFYGDTNPKDDWF

SEQ ID NO: 18 - protein, 30 aa, synthetic construct
  SITE 1..30: note = all D-amino acids
TKTSSSFEVRTWDPEGVIFYGDTNPKDDWF

SEQ ID NO: 19 - protein, 30 aa, synthetic construct
  SITE 1..30: note = all D-amino acids
TWDPEGVIFYGDTNPKDDWFMLGLRDGRPE

SEQ ID NO: 20 - protein, 29 aa, synthetic construct
  SITE 1..29: note = all D-amino acids
VMTFDLTKITKTSSSFEVRTWDPEGVIFY

SEQ ID NO: 21 - DNA, 20 bp, synthetic construct
  source 1..20: mol_type = other DNA
cccgaccaaggactttgttg

SEQ ID NO: 22 - DNA, 20 bp, synthetic construct
  source 1..20: mol_type = other DNA
ggcatcattgggcactcctt

SEQ ID NO: 23 - protein, 10 aa, synthetic construct
  SITE 1..10: note = all D-amino acids
TWDPEGVIFY

SEQ ID NO: 24 - protein, 9 aa, synthetic construct
  SITE 1..9: note = all D-amino acids
WDPEGVIFY

SEQ ID NO: 25 - protein, 9 aa, synthetic construct
  SITE 1..9: note = all D-amino acids
TDPEGVIFY

SEQ ID NO: 26 - protein, 9 aa, synthetic construct
  SITE 1..9: note = all D-amino acids
TWPEGVIFY

SEQ ID NO: 27 - protein, 9 aa, synthetic construct
  SITE 1..9: note = all D-amino acids
TWDEGVIFY

SEQ ID NO: 28 - protein, 9 aa, synthetic construct
  SITE 1..9: note = all D-amino acids
TWDPGVIFY

SEQ ID NO: 29 - protein, 9 aa, synthetic construct
  SITE 1..9: note = all D-amino acids
TWDPEVIFY

SEQ ID NO: 30 - protein, 9 aa, synthetic construct
  SITE 1..9: note = all D-amino acids
TWDPEGIFY

SEQ ID NO: 31 - protein, 9 aa, synthetic construct
  SITE 1..9: note = all D-amino acids
TWDPEGVFY

SEQ ID NO: 32 - protein, 9 aa, synthetic construct
  SITE 1..9: note = all D-amino acids
TWDPEGVIY

SEQ ID NO: 33 - protein, 9 aa, synthetic construct
  SITE 1..9: note = all D-amino acids
TWDPEGVIF

SEQ ID NO: 34 - protein, 8 aa, synthetic construct
  SITE 1..8: note = all D-amino acids
DPEGVIFY

SEQ ID NO: 35 - protein, 8 aa, synthetic construct
  SITE 1..8: note = all D-amino acids
WDPEGVIY

SEQ ID NO: 36 - protein, 8 aa, synthetic construct
  SITE 1..8: note = all D-amino acids
WDPEGVIF

SEQ ID NO: 37 - protein, 8 aa, synthetic construct
  SITE 1..8: note = all D-amino acids
TDPEGVIY

SEQ ID NO: 38 - protein, 8 aa, synthetic construct
  SITE 1..8: note = all D-amino acids
TDPEGVIF

SEQ ID NO: 39 - protein, 8 aa, synthetic construct
  SITE 1..8: note = all D-amino acids
TWDPEGVI

SEQ ID NO: 40 - protein, 7 aa, synthetic construct
  SITE 1..7: note = all D-amino acids
DPEGVFY

SEQ ID NO: 41 - protein, 7 aa, unidentified
  SITE 1..7: note = all D-amino acids
DPEGVIY

SEQ ID NO: 42 - protein, 7 aa, synthetic construct
  SITE 1..7: note = all D-amino acids
DPEGVIF

SEQ ID NO: 43 - protein, 7 aa, synthetic construct
  SITE 1..7: note = all D-amino acids
WDPEGVI

SEQ ID NO: 44 - protein, 7 aa, synthetic construct
  SITE 1..7: note = all D-amino acids
WDPEGVY

SEQ ID NO: 45 - protein, 7 aa, synthetic construct
  SITE 1..7: note = all D-amino acids
WDPEGVF

SEQ ID NO: 46 - protein, 6 aa, synthetic construct
  SITE 1..6: note = All D-amino acids
DPEGVI

SEQ ID NO: 47 - protein, 6 aa, synthetic construct
  SITE 1..6: note = all D-amino acids
WDPEGV

SEQ ID NO: 48 - protein, 6 aa, synthetic construct
  SITE 1..6: note = all D-amino acids
TWDPEG

SEQ ID NO: 49 - protein, 5 aa, synthetic construct
  SITE 1..5: note = all D-amino acids
WDPEG

SEQ ID NO: 50 - protein, 7 aa, synthetic construct
  SITE 1..7: note = all D-amino acids
WDPEGLY

SEQ ID NO: 51 - protein, 7 aa, synthetic construct
  SITE 1..7: note = all D-amino acids
DPEGLFY

SEQ ID NO: 52 - protein, 7 aa, synthetic construct
  SITE 1..7: note = all D-amino acids
DPEGVLY

SEQ ID NO: 53 - protein, 7 aa, synthetic construct
  SITE 1..7: note = all D-amino acids
WDNEGLY

SEQ ID NO: 54 - protein, 7 aa, synthetic construct
  SITE 1..7: note = all D-amino acids
NDPEGLY

SEQ ID NO: 55 - protein, 8 aa, synthetic construct
  SITE 1..8: note = all D-amino acids
NDPEGVIY

SEQ ID NO: 56 - protein, 6 aa, synthetic construct
  SITE 1..6: note = all D-amino acids
NDPEGY

SEQ ID NO: 57 - protein, 7 aa, synthetic construct
NDPEGLY

SEQ ID NO: 58 - protein, 7 aa, synthetic construct
  SITE 1: note = D-Asparagine
NDPEGLY

SEQ ID NO: 59 - protein, 7 aa, synthetic construct
  SITE 2: note = D-Aspartic Acid
NDPEGLY

SEQ ID NO: 60 - protein, 7 aa, synthetic construct
  SITE 3: note = D-Proline
NDPEGLY

SEQ ID NO: 61 - protein, 7 aa, synthetic construct
  SITE 4: note = D-Glutamic Acid
NDPEGLY

SEQ ID NO: 62 - protein, 7 aa, synthetic construct
  SITE 6: note = D-Leucine
NDPEGLY

SEQ ID NO: 63 - protein, 7 aa, synthetic construct
  SITE 7: note = D-Tyrosine
NDPEGLY

SEQ ID NO: 64 - DNA, 20 bp, synthetic construct
  source 1..20: mol_type = other DNA
gctgcaagcacagcctctct

SEQ ID NO: 65 - DNA, 20 bp, synthetic construct
  source 1..20: mol_type = other DNA
gaatgctggacaacatcctg

SEQ ID NO: 66 - DNA, 21 bp, synthetic construct
  source 1..21: mol_type = other DNA
gttcattagacatgcctcctg

SEQ ID NO: 67 - protein, 7 aa, synthetic construct
  SITE 1..7: note = All D-amino acids
NDVEGLY

SEQ ID NO: 68 - protein, 7 aa, synthetic construct
  SITE 1..7: note = All D-amino acids
NDAEGLY

SEQ ID NO: 69 - protein, 7 aa, synthetic construct
  SITE 1..7: note = All D-amino acids
NDLEGVY

SEQ ID NO: 70 - protein, 7 aa, synthetic construct
  SITE 1..7: note = All D-amino acids
NDLEGAY

SEQ ID NO: 71 - protein, 7 aa, synthetic construct
  SITE 1..7: note = All D-amino acids
NDLEGPY

SEQ ID NO: 72 - protein, 7 aa, synthetic construct
  SITE 1..7: note = all D-amino acids
NDEGLFY

SEQ ID NO: 73 - protein, 7 aa, synthetic construct
  SITE 1..7: note = All D-amino acids
NDLEGLY

SEQ ID NO: 74 - protein, 6 aa, synthetic construct
  SITE 1..6: note = all D-amino acids
NDEGLY

SEQ ID NO: 75 - protein, 6 aa, synthetic construct
  SITE 1..6: note = All D-amino acids
NDEGLY

SEQ ID NO: 76 - protein, 6 aa, synthetic construct
  SITE 1..6: note = All D-amino acids
NDEGLY

SEQ ID NO: 77 - DNA, 21 bp, synthetic construct
  source 1..21: mol_type = other DNA
tcttaactgccggatccacaa

SEQ ID NO: 78 - protein, 9 aa, synthetic construct
  REGION 1..9: note = each amino acid being L- or D-amino acid
TWPEGVIFY

SEQ ID NO: 79 - protein, 9 aa, synthetic construct
  REGION 1..9: note = Each amino acid being L- or D-amino acid
TWDEGVIFY

SEQ ID NO: 80 - protein, 9 aa, synthetic construct
  REGION 1..9: note = Each amino acid being L- or D-amino acid
TWDPGVIFY

SEQ ID NO: 81 - protein, 9 aa, synthetic construct
  PROPEP 1..9: note = Each amino acid being L- or D-amino acid
TWDPEVIFY

SEQ ID NO: 82 - protein, 10 aa, synthetic construct
  REGION 1..10: note = each amino acid being L-or D-amino acid, provided that at least one of the amino acids is D-amino acid
  MOD_RES 1..10: note = Amidation
TWDPEGVIFY

SEQ ID NO: 83 - protein, 10 aa, synthetic construct
  REGION 1..10: note = Each amino acid being L- or D-amino acid
  VARIANT 1: note = It can be deleted. It can be represented as [T]a wherein a is 0 or 1.
  VARIANT 2: note = X represents W, N, Q or K and it can be deleted. It can be represented as [X2]b wherein X2 represents W, N, Q or K and b is 0 or 1.
  VARIANT 4: note = X represents P, L or N
  VARIANT 7: note = X represents A, V, L or I and it can be deleted. It can be represented as [X6]d wherein X6 is A, V, L or I and d is 0 or 1.
  VARIANT 8: note = X represents A, V, L or I and it can be deleted. It can be represented as [X7]e wherein X7 is A, V, L or I and e is 0 or 1.
  VARIANT 9: note = X represents F or P and it can be deleted. It can be represented as [X8]f wherein X8 is F or P and f is 0 or 1.
  VARIANT 10: note = It can be deleted. It can be represented as [Y]g wherein g is 0 or 1.
TXDXEGXXXY

SEQ ID NO: 84 - protein, 10 aa, synthetic construct
  VARIANT 1: note = It can be deleted. It can be represented as [T]a wherein a is 0 or 1.
  VARIANT 2: note = X represents W, N, Q or K and it can be deleted. It can be represented as [X2]b wherein X2 represents W, N, Q or K and b is 0 or 1.
  VARIANT 7: note = X represents A, V, L or I and it can be deleted. It can be represented as [X6]d wherein d is 0 or 1.
  VARIANT 8: note = X represents A, V, L or I and it can be deleted. It can be represented as [X7]e wherein e is 0 or 1
  VARIANT 9: note = X represents F or P and it can be deleted. It can be represented as [X8]f wherein X8 represents F or P and f is 0 or 1.
  VARIANT 10: note = It can be deleted. It can be represented as [Y]g wherein g is 0 or 1.
  REGION 1..10: note = Each amino acid being L- or D-amino acid.
TXDPEGXXXY

SEQ ID NO: 85 - protein, 10 aa, synthetic construct
  VARIANT 1: note = It can be deleted. It can be represented as [T]a wherein a is 0 or 1.
  VARIANT 2: note = X represents W, N, Q or K and it can be deleted. It can be represented as [X2]b wherein X2 represents W, N, Q or K and b is 0 or 1.
  VARIANT 7: note = X represents A, V, L or I and it can be deleted. It can be represented as [X6]d wherein X6 represents A, V, L or I and d is 0 or 1.
  VARIANT 8: note = X represents A, V, L or I and it can be deleted. It can be represented as [X7]e wherein X7 represents A, V, L or I and e is 0 or 1.
  VARIANT 9: note = X represents F or P and it can be deleted. It can be represented as [X8]f wherein X8 represents F or P and f is 0 or 1.
  VARIANT 10: note = It can be deleted. It can be represented as [Y]g wherein g is 0 or 1.
  REGION 1..10: note = Each amino acid being L- or D-amino acid.
TXDLEGXXXY

SEQ ID NO: 86 - protein, 10 aa, synthetic construct
  VARIANT 1: note = It can be deleted. It can be represented as [T]a wherein a is 0 or 1.
  VARIANT 2: note = X represents W, N, Q or K and it can be deleted. It can be represented as [X2]b wherein X2 represents W, N, Q or K and b is 0 or 1.
  VARIANT 7: note = X represents A, V, L or I and it can be deleted. It can be represented as [X6]d wherein X6 represents A, V, L or I and d is 0 or 1.
  VARIANT 8: note = X represents A, V, L or I and it can be deleted. It can be represented as [X7]e wherein X7 represents A, V, L or I and d is 0 or 1.
  VARIANT 9: note = X represents F or P and it can be deleted. It can be represented as [X8]f wherein X8 represents F or P and f is 0 or 1.
  VARIANT 10: note = It can be deleted. It can be represented as [Y]g wherein g is 0 or 1.
  REGION 1..10: note = Each amino acid being L- or D-amino acid.
TXDNEGXXXY

SEQ ID NO: 87 - protein, 10 aa, synthetic construct
  REGION 1..10: note = Each amino acid being L- or D-amino acid
  VARIANT 1: note = It can be deleted. It can be represented as [T]a wherein a is 0 or 1.
  VARIANT 2: note = X represents W, N, Q or K and it can be deleted. It can be represented as [X2]b wherein X2 represents W, N, Q or K and b is 0 or 1.
  VARIANT 4: note = X represents P, L or N.
  VARIANT 7: note = X represents A, V, L or I, and it can be deleted. It can be represented as [X6]d wherein X6 represents A, V, L or I, and d is 0 or 1.
  VARIANT 8: note = X represents A, V, L or I, and it can be deleted. It can be represented as [X7]e wherein X7 represents A, V, L or I, and e is 0 or 1.
  VARIANT 9: note = It can be deleted. It can be represented as [F]f wherein f is 0 or 1.
  VARIANT 10: note = It can be deleted. It can be represented as [Y]g wherein g is 0 or 1.
TXDXEGXXFY

SEQ ID NO: 88 - protein, 10 aa, synthetic construct
  REGION 1..10: note = Each amino acid being L- or D-amino acid.
  VARIANT 1: note = It can b deleted. It can be represented as [T]a wherein a is 0 or 1.e
  VARIANT 2: note = X represents W, N, Q or K and it can be deleted. It can be represented as [X2]b wherein X2 represents W, N, Q or K and b is 0 or 1.
  VARIANT 7: note = X represents A, V, L or I and it can be deleted. It can be represented as [X6]d wherein X6 represents A, V, L or I and d is 0 or 1.
  VARIANT 8: note = X represents A, V, L or I and it can be deleted. It can be represented as [X7]e wherein X7 represents A, V, L or I and e is 0 or 1.
  VARIANT 9: note = It can be deleted. It can be represented as [F]f wherein f is 0 or 1.
  VARIANT 10: note = It can be deleted. It can be represented as[Y]g wherein g is 0 or 1.
TXDPEGXXFY

SEQ ID NO: 89 - protein, 10 aa, synthetic construct
  REGION 1..10: note = each amino acid beeing L- or D-amino acid
  VARIANT 1: note = It can be deleted. It can be represented as [T]a wherein a is 0 or 1.
  VARIANT 2: note = X represents W, N, Q or K and it can be deleted. It can be represented as [X2]b wherein X2 represents W, N, Q or K and b is 0 or 1.
  VARIANT 7: note = X represents A, V, L or I and it can be deleted. It can be represented as [X6]d wherein X6 represents A, V, L or I and d is 0 or 1.
  VARIANT 8: note = X represents A, V, L or I and it can be deleted. It can be represented as [X7]e wherein X7 represents A, V, L or I and e is 0 or 1.
  VARIANT 9: note = It can be deleted. It can be represented as [F]f wherein f is 0 or 1.
  VARIANT 10: note = It can be deleted. It can be represented as [Y]g wherein g is 0 or 1.
TXDNEGXXFY

SEQ ID NO: 90 - protein, 10 aa, synthetic construct
  REGION 1..10: note = each amino acid being L- or D-amino acid
  VARIANT 1: note = It can be deleted. It can be represented as [T]a wherein a is 0 or 1.
  VARIANT 2: note = X represents W, N, Q or K and it can be deleted. It can be represented as [X2]b wherein X2 represents W, N, Q or K and b is 0 or 1.
  VARIANT 7: note = X represents A, V, L or I and it can be deleted. It can be represented as [X6]d wherein X6 represents A, V, L or I and d is 0 or 1.
  VARIANT 8: note = X represents A, V, L or I and it can be deleted. It can be represented as [X7]e wherein X7 represents A, V, L or I and e is 0 or 1.
  VARIANT 9: note = It can be deleted. It can be represented as [F]f wherein f is 0 or 1.
  VARIANT 10: note = It can be deleted. It can be represented as [Y]g wherein g is 0 or 1.
TXDLEGXXFY

SEQ ID NO: 91 - protein, 10 aa, synthetic construct
  REGION 1..10: note = each amino acid being L- or D-amino acid
  VARIANT 1: note = X represents T or A and it can be deleted. It can be represented as [X1]a wherein X1 represents T or A and a is 0 or 1.
  VARIANT 2: note = X represents K or A and it can be deleted. It can be represented as [X2]b wherein X2 represents K or A and b is 0 or 1.
  VARIANT 3: note = X represents T or A and it can be deleted. It can be represented as [X3]c wherein X3 represents T or A and c is 0 or 1.
  VARIANT 4: note = X represents S or A and it can be deleted. It can be represented as [X4]d wherein X4 represents S or A and d is 0 or 1.
  VARIANT 5: note = X represents S or A and it can be deleted. It can be represented as [X5]e wherein X5 represents S or A and e is 0 or 1.
  VARIANT 8: note = X represents E or Q.
XXXXXSFXVR

SEQ ID NO: 92 - protein, 10 aa, synthetic construct
  REGION 1..10: note = each amino acid being L- or D- amino acid
  MOD_RES 1: note = Acetylation - the N-terminus is NHC(O)CH3
TKTSSSFEVR

SEQ ID NO: 93 - protein, 10 aa, synthetic construct
  REGION 1..10: note = each amino acid being L- or D-amino acid
  MOD_RES 1: note = palmitoylated - the N-terminus is -NHC(O)(CH2)14CH3
TKTSSSFEVR

SEQ ID NO: 94 - protein, 10 aa, synthetic construct
  REGION 1..10: note = each amino acid being L- or D-amino acid
TKTSSSFQVR

SEQ ID NO: 95 - protein, 10 aa, synthetic construct
  REGION 1..10: note = each amino acid being L- or D-amino acid
  MOD_RES 2: note = Acetylated Lysine
TKTSSSFEVR

SEQ ID NO: 96 - protein, 10 aa, synthetic construct
  REGION 1..10: note = each amino acid being L- or D- amino acid
  MOD_RES 2: note = Palmitoylated Lysine
TKTSSSFEVR

SEQ ID NO: 97 - protein, 9 aa, synthetic construct
  REGION 1..9: note = each amino acid being L- or D-amino acid
KTSSSFEVR

SEQ ID NO: 98 - protein, 8 aa, synthetic construct
  REGION 1..8: note = each amino acid being L- or D-amino acid
TSSSFEVR

SEQ ID NO: 99 - protein, 7 aa, synthetic construct
  REGION 1..7: note = each amino acid being L- or D-amino acid
SSSFEVR

SEQ ID NO: 100 - protein, 6 aa, synthetic construct
  REGION 1..6: note = each amino acid being L- or D-amino acid
SSFEVR

SEQ ID NO: 101 - protein, 5 aa, synthetic construct
  REGION 1..5: note = each amino acid being L-or D-amino acid
SFEVR

SEQ ID NO: 102 - protein, 10 aa, synthetic construct
  REGION 1..10: note = each amino acid being L- or D- amino acid
AKTSSSFEVR

SEQ ID NO: 103 - protein, 10 aa, synthetic construct
  REGION 1..10: note = each amino acid being L- or D- amino acid
TATSSSFEVR

SEQ ID NO: 104 - protein, 10 aa, synthetic construct
  REGION 1..10: note = each amino acid being L- or D- amino acid
TKASSSFEVR

SEQ ID NO: 105 - protein, 10 aa, synthetic construct
  REGION 1..10: note = each amino acid being L- or D-amino acid
TKTASSFEVR

SEQ ID NO: 106 - protein, 10 aa, synthetic construct
  REGION 1..10: note = each amino acid being L- or D-amino acid
TKTSASFEVR

SEQ ID NO: 107 - protein, 10 aa, synthetic construct
  REGION 1..10: note = each amino acid is L- or D-amino acid
  MOD_RES 10: note = Amidation - The C-terminus is CONH2
TWDPEGVIFY

SEQ ID NO: 108 - protein, 10 aa, synthetic construct
  REGION 1..10: note = each amino acid being L- or D-amino acid
  VARIANT 1: note = X represents T, or A and it can be deleted. It can be represented as [X1]a wherein X1 represents T, or A and a is 0 or 1.
  VARIANT 2: note = X represents W, N, or A.
  VARIANT 3: note = X represents D or N.
  VARIANT 4: note = X represents P, or L and it can be deleted. It can be represented as [X4]c wherein X4 represents P, or L and c is 0 or 1.
  VARIANT 5: note = X represents E or Q.
  VARIANT 7: note = It can be deleted. It can be represented as [V]d wherein d is 0 or 1.
  VARIANT 8: note = X represents L or I
  VARIANT 9: note = It can be deleted. It can be represented as [F]f wherein f is 0 or 1.
XXXXXGVXFY

SEQ ID NO: 109 - protein, 10 aa, synthetic construct
  REGION 1..10: note = each amino acid is L- or D-amino acid
TWNPEGVIFY

SEQ ID NO: 110 - protein, 10 aa, synthetic construct
  REGION 1..10: note = each amino acid being L- or D-amino acid
TWDPQGVIFY

SEQ ID NO: 111 - protein, 10 aa, synthetic construct
  REGION 1..10: note = each amino acid being L- or D- amino acid
TNDPEGVIFY

SEQ ID NO: 112 - protein, 10 aa, synthetic construct
  REGION 1..10: note = each amino acid being L- or D-amino acid
  MOD_RES 1: note = Acetylated Tryptophan
TWDPEGVIFY

SEQ ID NO: 113 - protein, 10 aa, synthetic construct
  REGION 1..10: note = each amino acid being L- or D-amino acid
  MOD_RES 2: note = Palmitoylated Tryptophan
TWDPEGVIFY

SEQ ID NO: 114 - protein, 10 aa, synthetic construct
  REGION 1..10: note = each amino acid being L- or D-amino acid
TWDPEGVLFY

SEQ ID NO: 115 - protein, 10 aa, synthetic construct
TWDPEGVLFY

SEQ ID NO: 116 - protein, 10 aa, synthetic construct
  REGION 1..10: note = each amino acid being L- or D-amino acid
AWDPEGVLFY

SEQ ID NO: 117 - protein, 10 aa, synthetic construct
  REGION 1..10: note = each amino acid being L- or D-amino acid
TADPEGVLFY

SEQ ID NO: 118 - protein, 10 aa, synthetic construct
  REGION 1..10: note = each amino acid being L- or D-amino acid
TNDPEGVLFY

SEQ ID NO: 119 - protein, 10 aa, synthetic construct
  REGION 1..10: note = each amino acid being L- or D-amino acid
ANDPEGVLFY

SEQ ID NO: 120 - protein, 9 aa, synthetic construct
  REGION 1..9: note = each amino acid being L- or D-amino acid
NDPEGVLFY

SEQ ID NO: 121 - protein, 7 aa, synthetic construct
  REGION 1..7: note = each amino acid being L- or D-amino acid
NDEGLFY

SEQ ID NO: 122 - protein, 7 aa, synthetic construct
  REGION 1..7: note = each amino acid being L- or D-amino acid
NDPEGLY

SEQ ID NO: 123 - protein, 7 aa, synthetic construct
  REGION 1..7: note = each amino acid being L- or D-amino acid
NDLEGLY

SEQ ID NO: 124 - protein, 7 aa, synthetic construct
NDLEGLY

SEQ ID NO: 125 - DNA, 20 bp, synthetic construct
  source 1..20: mol_type = other DNA
agggttcgattccggagagg

SEQ ID NO: 126 - DNA, 22 bp, synthetic construct
  source 1..22: mol_type = other DNA
caactttaatatacgctattgg

SEQ ID NO: 127 - protein, 20 aa, synthetic construct
  SITE 1..20: note = AllD-amino acids
  MOD_RES 1: note = Acetylated - the N-terminus is NHC(O)CH3
TKTSSSFEVRTWDPEGVIFY

SEQ ID NO: 128 - protein, 20 aa, synthetic construct
  SITE 1..20: note = all D-amino acids
  MOD_RES 1..20: note = palmitoylated - the N-terminus is -NHC(O)(CH2)14CH3
TKTSSSFEVRTWDPEGVIFY

SEQ ID NO: 129 - protein, 20 aa, synthetic construct
  SITE 1..20: note = all D-amino acids
  MOD_RES 20: note = Amidation - The C-terminus is CONH2
TKTSSSFEVRTWDPEGVIFY

SEQ ID NO: 130 - protein, 20 aa, synthetic construct
  SITE 1..20: note = all D-amino acids
  MOD_RES 20: note = The C-terminus is -CONHCH3
TKTSSSFEVRTWDPEGVIFY

SEQ ID NO: 131 - protein, 20 aa, synthetic construct
  SITE 1..20: note = all D-amino acids
TKTSSSFEVRTWNPEGVIFY

SEQ ID NO: 132 - protein, 20 aa, synthetic construct
  SITE 1..20: note = all D-amino acids
TKTSSSFEVRTWDPQGVIFY

SEQ ID NO: 133 - protein, 20 aa, synthetic construct
  SITE 1..20: note = all D-amino acids
TKTSSSFEVRTNDPEGVIFY

SEQ ID NO: 134 - protein, 20 aa, synthetic construct
  SITE 1..20: note = all D-amino acids
TKTSSSFQVRTWDPEGVIFY

SEQ ID NO: 135 - protein, 20 aa, synthetic construct
  SITE 1..20: note = all D-amino acids
  MOD_RES 2: note = acetylated Lysine
TKTSSSFEVRTWDPEGVIFY

SEQ ID NO: 136 - protein, 20 aa, synthetic construct
  SITE 1..20: note = all D-amino acids
  MOD_RES 12: note = Acetylated Tryptophan
TKTSSSFEVRTWDPEGVIFY

SEQ ID NO: 137 - protein, 20 aa, synthetic construct
  SITE 1..20: note = all D-amino acids
  MOD_RES 2: note = Palmitoylated Lysine
TKTSSSFEVRTWDPEGVIFY

SEQ ID NO: 138 - protein, 20 aa, synthetic construct
  SITE 1..20: note = all D-amino acids
  MOD_RES 12: note = Palmitoylated Tryptophan
TKTSSSFEVRTWDPEGVIFY

SEQ ID NO: 139 - protein, 20 aa, synthetic construct
  SITE 1..20: note = all D-amino acids
TKTSSSFEVRTWDPEGVLFY

SEQ ID NO: 140 - protein, 19 aa, synthetic construct
  SITE 1..19: note = all D-amino acids
KTSSSFEVRTWDPEGVIFY

SEQ ID NO: 141 - protein, 18 aa, synthetic construct
  SITE 1..18: note = all D-amino acids
TSSSFEVRTWDPEGVIFY

SEQ ID NO: 142 - protein, 17 aa, synthetic construct
  SITE 1..17: note = all D-amino acids
SSSFEVRTWDPEGVIFY

SEQ ID NO: 143 - protein, 16 aa, synthetic construct
  SIGNAL 1..16: note = all D-amino acids
SSFEVRTWDPEGVIFY

SEQ ID NO: 144 - protein, 15 aa, synthetic construct
  SITE 1..15: note = all D-amino acids
SFEVRTWDPEGVIFY

SEQ ID NO: 145 - protein, 20 aa, synthetic construct
  SITE 1..20: note = all D-amino acids
AKTSSSFEVRTWDPEGVIFY

SEQ ID NO: 146 - protein, 20 aa, synthetic construct
  SITE 1..20: note = all D-amino acids
TATSSSFEVRTWDPEGVIFY

SEQ ID NO: 147 - protein, 20 aa, synthetic construct
  SITE 1..20: note = all D-amino acids
TKASSSFEVRTWDPEGVIFY

SEQ ID NO: 148 - protein, 20 aa, synthetic construct
  SITE 1..20: note = all D-amino acids
TKTASSFEVRTWDPEGVIFY

SEQ ID NO: 149 - protein, 20 aa, synthetic construct
  SITE 1..20: note = all D-amino acids
TKTSASFEVRTWDPEGVIFY

SEQ ID NO: 150 - protein, 20 aa, synthetic construct
  SITE 1..20: note = all D-amino acids
TKTSSSFEVRTWDPEGVLFY

SEQ ID NO: 151 - protein, 20 aa, synthetic construct
TKTSSSFEVRTWDPEGVLFY

SEQ ID NO: 152 - protein, 20 aa, synthetic construct
  SITE 1..20: note = all D-amino acids
TKTSSSFEVRAWDPEGVLFY

SEQ ID NO: 153 - protein, 20 aa, synthetic construct
  SITE 1..20: note = all D-amino acids
TKTSSSFEVRTADPEGVLFY

SEQ ID NO: 154 - protein, 20 aa, synthetic construct
  SITE 1..20: note = all D-amino acids
TKTSSSFEVRTNDPEGVLFY

SEQ ID NO: 155 - protein, 20 aa, synthetic construct
  SITE 1..20: note = all D-amino acids
TKTSSSFEVRAWDPEGVLFY

SEQ ID NO: 156 - protein, 20 aa, synthetic construct
  SITE 1..20: note = all D-amino acids
TKTSSSFEVRANDPEGVLFY

SEQ ID NO: 157 - protein, 19 aa, synthetic construct
  SITE 1..19: note = all D-amino acids
TKTSSSFEVRNDPEGVLFY

SEQ ID NO: 158 - protein, 17 aa, synthetic construct
  SITE 1..17: note = all D-amino acids
TKTSSSFEVRNDEGLFY

SEQ ID NO: 159 - protein, 17 aa, synthetic construct
  SITE 1..17: note = all D-amino acids
TKTSSSFEVRNDPEGLY

SEQ ID NO: 160 - protein, 17 aa, synthetic construct
  SITE 1..17: note = all D-amino acids
TKTSSSFEVRNDLEGLY

SEQ ID NO: 161 - protein, 17 aa, synthetic construct
TKTSSSFEVRNDLEGLY

SEQ ID NO: 162 - DNA, 19 bp, synthetic construct
  source 1..19: mol_type = other DNA
gcccaaacctgatggcatt

SEQ ID NO: 163 - DNA, 25 bp, synthetic construct
  source 1..25: mol_type = other DNA
catcttctcaaaattcgagtgacaa

SEQ ID NO: 164 - DNA, 23 bp, synthetic construct
  source 1..23: mol_type = other DNA
tgggagtagacaaggtacaaccc

SEQ ID NO: 165 - DNA, 20 bp, synthetic construct
  source 1..20: mol_type = other DNA
agggctaccatgccaacttc

SEQ ID NO: 166 - DNA, 19 bp, synthetic construct
  source 1..19: mol_type = other DNA
ccacgtagtagacgatggg

SEQ ID NO: 167 - DNA, 23 bp, synthetic construct
  source 1..23: mol_type = other DNA
tcgtggaaatgagaaaagagttg

SEQ ID NO: 168 - DNA, 23 bp, synthetic construct
  source 1..23: mol_type = other DNA
agtgcatcatcgttgttcataca

SEQ ID NO: 169 - DNA, 22 bp, synthetic construct
  source 1..22: mol_type = other DNA
ctggtgtgtgacgttcccatta

SEQ ID NO: 170 - DNA, 18 bp, synthetic construct
  source 1..18: mol_type = other DNA
ccgacagcacgaggcttt

SEQ ID NO: 171 - DNA, 22 bp, synthetic construct
  source 1..22: mol_type = other DNA
ctttggctatgggcttccagtc

SEQ ID NO: 172 - DNA, 24 bp, synthetic construct
  source 1..24: mol_type = other DNA
gcaaggaggacagagtttatcgtg

SEQ ID NO: 173 - DNA, 19 bp, synthetic construct
  source 1..19: mol_type = other DNA
gctcctcttaggggccact

SEQ ID NO: 174 - DNA, 20 bp, synthetic construct
  source 1..20: mol_type = other DNA
attggggacccttaggccat

SEQ ID NO: 175 - DNA, 19 bp, synthetic construct
  source 1..19: mol_type = other DNA
gagacccctgtgtcggttc

SEQ ID NO: 176 - DNA, 22 bp, synthetic construct
  source 1..22: mol_type = other DNA
ctgcgtgtgtgaaatgtcattg

SEQ ID NO: 177 - DNA, 21 bp, synthetic construct
  source 1..21: mol_type = other DNA
tgttcctcttaatcctgccca

SEQ ID NO: 178 - DNA, 21 bp, synthetic construct
  source 1..21: mol_type = other DNA
ccaacctgcacaagttccctt

SEQ ID NO: 179 - protein, 10 aa, synthetic construct
  REGION 1..10: note = each amino acid being L- or D-amino acid
  MOD_RES 10: note = The C-terminus is -CONHCH3
TWDPEGVIFY

SEQ ID NO: 180 - protein, 21 aa, synthetic construct
  REGION 1..21: note = each one of the amino acids being L- or D-amino acid.
ATKTSASFEVRTWDPEAVIFY

SEQ ID NO: 181 - protein, 30 aa, synthetic construct
  REGION 1..30: note = each one of the amino acids being L- or D-amino acid
TKTASAFEVRTWDPEGVIFYGDTNPKDDWF

SEQ ID NO: 182 - protein, 20 aa, synthetic construct
  REGION 1..20: note = each one of the amino acids being L- or D-amino acid
TKTASAFEVRTWDPEGVIFY

SEQ ID NO: 183 - protein, 30 aa, synthetic construct
  REGION 1..30: note = each one of the amino acids being L- or D-amino acid
TKTSASFEVRTWDPEGVIFYADTNPKDDWF

(19) United States
(12) Patent Application Publication
(10) Pub. No.: US 2026/0297136 A1
(43) Pub. Date: Oct. 1, 2026

(54) PEPTIDES WITH LIPOLYTIC, ANTI-LIPOGENIC, ANTIFIBROTIC AND ANTI-INFLAMMATORY ACTIVITY

(71) Applicants: FUNDACIÓ HOSPITAL UNIVERSITARI VALL D'HEBRON - INSTITUT DE RECERCA, BARCELONA (ES); ENDOLIPID THERAPEUTICS, S.L., BARCELONA (ES)

(72) Inventors: DAVID MARTINEZ SELVA, BARCELONA (ES); RAFAEL SIMÓ CANONGE, SANT CUGAT DEL VALLÈS (ES)

(21) Appl. No.: 19/489,417

(22) PCT Filed: Jun. 1, 2024
(86) PCT No.: PCT/EP2024/065136

§ 371 Date: Dec. 2, 2025

(30) Foreign Application Priority Data
Jun. 2, 2023 (EP) 23382535.5

(51) Int. Cl. C07K 14/00; A61K 9/00; A61K 38/00; A61P 1/16; A61P 3/04

(52) U.S. Cl. CPC C07K 14/001; A61K 9/0019; A61P 1/16; A61P 3/04; A61K 38/00

(57) ABSTRACT

Peptides, or pharmaceutically acceptable salts thereof, the peptides having a length of 10 to 40 amino acids, each one including a peptide fragment F1 with SEQ ID NO. 9: TWDPEGVIFY or a sequence variant thereof having at least 80% sequence identity with SEQ ID NO.9. The peptides further include a peptide fragment F2 at the N-terminal end of peptide fragment F1, with SEQ ID NO. 13 (TKTSSSFEVR) or a sequence variant thereof with at least 80% sequence identity with SEQ ID NO.13; or SEQ ID NO. 14 (VMTFDLTKITKTSSSFEVR) or that is a sequence variant thereof with at least 80% sequence identity with SEQ ID NO.14; and/or a peptide fragment F3 at the C-terminal end of peptide fragment F1, with SEQ ID NO. 4 (GDTNPKDDWF) or a variant thereof with at least 80% sequence identity with SEQ ID NO.4; or with SEQ ID NO. 15 (GDTNPKDDWFMLGLRDGRPE) or a variant thereof with at least 80% sequence identity with SEQ ID NO.15.

This application claims the benefit of European Patent Application no. EP23382535.5 filed on 2 Jun. 2023.

### TECHNICAL FIELD

The present invention relates to the field of peptides and their medical use in therapy of diseases associated to the accumulation of fat in an organ, to inflammation and/or fibrosis, in particular in the therapy of Non-alcoholic fatty liver disease (NAFLD) and obesity. It also relates to their use in cosmetics.

### BACKGROUND ART

Non-alcoholic fatty liver disease (NAFLD) is a leading cause of liver-related morbidity and mortality. It affects between 17% and 46% of adults. It is characterized by the accumulation of fat inside the liver cells, which causes inflammation and scarring of the organ.

NAFLD encompasses a spectrum of conditions which ranges from fatty liver to cirrhosis. The main stages of NAFLD are (i) fatty liver (steatosis), (ii) non-alcoholic steatohepatitis (NASH), (iii) hepatic fibrosis, and (iv) cirrhosis.

Fatty liver is characterized by an excess of fat in the liver but very little or no inflammation or cell damage. In NASH, accumulation of fat is accompanied by inflammation and liver cells damage. Inflammation and cell damage can lead to scarring and the appearance of fibrosis. Thus, persistent inflammation can cause scar tissue formation and hepatic fibrosis, which can ultimately lead to cirrhosis. Thus, NAFLD ha a complex pathophysiology, that includes lipid accumulation, inflammation and fibrosis.

The well-known primary causes of NAFLD are obesity, type II diabetes, dyslipidemia, and insulin resistance. Interestingly, increases in adipocyte volume and tissue mass due to obesity can result in inflammation, further dysregulation in adipose tissue function, and eventually adipose tissue fibrosis.

The main treatment of NAFLD consist in reducing corporal weight. Weight loss can reduce fat, inflammation, and fibrosis—or scarring—in the liver. However, apart from controlling the conditions that contribute to fatty liver disease, efficient treatments of NASH are missing. In EP 3003351 A1 it is shown that sex hormone-binding globulin (SHBG) protein has lipolytic and anti-lipogenic activity, and that it can be useful for the treatment of hepatic steatosis or obesity. However, the specific region of the protein that could be used as a safer, and easier to synthesize, active ingredient is not shown.

Therefore, there is a need in the art for efficient treatments of complex diseases that involve lipid accumulation, inflammation and fibrosis, such as, NAFLD or obesity.

### SUMMARY OF THE INVENTION

the authors of the present invention have identified short length peptides that have anti-lipogenic, anti-fibrotic, lipolytic and anti-inflammatory activity and that can be useful for the prevention and treatment of diseases characterized in an increased lipogenesis, fibrosis, and inflammation, such as non-alcoholic fatty liver disease (NAFLD), obesity, lipohyperdystrophies, inflammatory diseases, or fibrosis.

Thus, in a first aspect, the invention relates to a peptide or a pharmaceutically acceptable salt thereof, wherein the peptide has a length of 9 to 49 amino acids, each one of the amino acids being L- or D-amino acid and comprises:
a peptide fragment F1 with a length of 4-10 amino acids, each one of the amino acids being L- or D-amino acid, and with sequence

SEQ ID NO. 1:
(X¹)ₐ(X²)_{b}(X³)(X⁴)_{c}(X⁵)G(X⁶)_{d}(X⁷)ₑ(X⁸)_{f}(Y)_{g}

wherein:
X¹ represents T, or A,
X² represents W, N, Q, K or A,
X³ represents D or N,
X⁴ represents P, N, a basic amino acid or a hydrophobic amino acid, preferably P, N, L, V, or A,
X⁵ represents E or Q,
X⁶ and X⁷ are the same or different and represent A, V, L or I,
X⁸ represents F or P,
subindexes “a”, “b”, “c”, “d”, “e”, “f” and “g” are integer numbers independently selected from 0 and 1 and at least two of them represent 1,
wherein the peptide further comprises:
   i) a peptide fragment F2 at the N-terminal end of peptide fragment F1, with a length of 5 to 19 amino acids, each one of the amino acids being L- or D-amino acid, and that comprises sequence SEQ ID NO.2 (SF(X⁹)VR), wherein X⁹ represents E or Q, and/or
   ii) a peptide fragment F3 at the C-terminal end of peptide fragment F1, with a length of 10 to 20 amino acids, each one of the amino acids being L- or D-amino acid, and that comprises SEQ ID NO. 4 (GDTNPKDDWF),
   particularly, wherein the peptide has a length of 10-40 amino acids, and consists of a peptide fragment F1 and, additionally: of a peptide fragment F2 at the N-terminal end of peptide fragment F1 and/or of a peptide fragment F3 at the C-terminal end of peptide fragment F1, wherein:
   i) peptide fragment F1 has SEQ ID NO. 9: TWDPEGVIFY or is a variant thereof that has at least 80% sequence identity with SEQ ID NO.9, wherein each one of the amino acids of peptide fragment F1 is a L- or D-amino acid;
   ii) peptide fragment F2 has SEQ ID NO. 13 (TKTSSSFEVR) or is a variant thereof with at least 80% sequence identity with SEQ ID NO.13; or has SEQ ID NO. 14 (VMTFDLTKITKTSSSFEVR) or is a variant thereof with at least 80% sequence identity with SEQ ID NO.14; and wherein each one of the amino acids of peptide fragment F2 is a L- or D-amino acid;
   and
   iii) peptide fragment F3 has SEQ ID NO. 4 (GDTNPKDDWF) or is a variant thereof with at least 80% sequence identity with SEQ ID NO.4; or has SEQ ID NO. 15 (GDTNPKDDWFMLGLRDGRPE) or is a variant thereof with at least 90% sequence identity with SEQ ID NO.15; and wherein each one of the amino acids of peptide fragment F3 is a L- or D-amino acid.

The examples below show that peptides falling within the scope of the first aspect have the advantageous effects mentioned above (lipolytic, anti-lipogenic, antifibrotic and anti-inflammatory). For example, peptide with SEQ ID NO. 16 reduces the mRNA expression of key hepatic lipogenic enzymes in HepG2 cells, reduces collagen fibrosis (collagen 1A expression) induced by TGFβ1 in LX-2 cells, and induces lipolysis in adipocytes (see example 3).

Furthermore, as disclosed in Example 4, mice that received subcutaneous administration of the peptide with SEQ ID NO.16 while being treated to develop obesity and fatty liver disease, showed a reduction of total body weight gain, liver weight, subcutaneous and visceral adipose tissue, of the expression of different indicators of hepatic lipogenesis and of hepatic lipid accumulation, reduced steatosis scores, and reduced hepatic inflammation as compared to control mice simply treated to develop obesity and fatty liver disease but with no SEQ ID NO. 16 peptide administration. Thus, the authors of the present invention have shown that peptide with SEQ ID NO. 16, prevents the development of symptoms associated to obesity and NAFLD in an animal model of the diseases, including lipid accumulation, fibrosis and inflammation.

As disclosed in Example 6, a mouse model of obesity and fatty liver disease that received subcutaneous administration of SEQ ID NO. 16 showed a reduction of body weight gain, of liver weight, of serum levels of glucose, Alanine transaminase (ALT) and Aspartate transaminase (AST), of hepatic lipid accumulation and of hepatic lipogenesis indicators, of steatosis scores, as well as a reduction in hepatic and adipose tissue inflammation as compared to obesity and fatty liver disease mouse model that did not receive SEQ ID NO. 16 administration. In addition, mouse model that received SEQ ID NO. 16 administration did not show hepatic fibrosis, contrary to those with no SEQ ID NO. 16 administration. Thus, the authors of the present invention have shown that peptide with SEQ ID NO. 16 can revert the symptomatology associated to obesity and NAFLD in an animal model of the diseases, in particular lipid accumulation, fibrosis and inflammation.

Furthermore, the authors have found that subcutaneous administration of peptide with SEQ ID NO. 16 to a mouse model of fibrosis reduces the symptomatology of the disease.

In a second aspect, the invention relates to a pharmaceutical composition comprising a therapeutical effective amount of the peptide, or pharmaceutically acceptable salt thereof, according to the first aspect of the invention, and one or more therapeutically acceptable carriers.

In a third aspect, the invention relates to the peptide, or a pharmaceutically acceptable salt thereof, according to the first aspect of the invention, or the pharmaceutical composition according to the second aspect of the invention, for use in therapy.

In a fourth aspect, the invention relates to the peptide, or a pharmaceutically acceptable salt thereof, according to the first aspect of the invention, or the pharmaceutical composition according to the second aspect of the invention, for use in the treatment and/or prevention of a condition or disease caused or associated to the accumulation of fat in an organ or tissue, particularly in an organ, inflammation and/or fibrosis.

In a fifth aspect, the invention relates to a cosmetic composition comprising the peptide, or pharmaceutically acceptable salt thereof, of the first aspect of the invention, and one or more cosmetically acceptable carriers.

In a sixth aspect, the invention relates to the use of the peptide or a pharmaceutically acceptable salt thereof, according to the first aspect of the invention, or the cosmetic composition according to the fifth aspect of the invention, in cosmetics.

In a seventh aspect, the invention relates to a kit of parts comprising:
a peptide, or pharmaceutically acceptable salt thereof, according to the first aspect of the invention, the pharmaceutical composition according to the second aspect of the invention, or the cosmetic composition according to the fifth aspect of the invention, and
a device selected from the group consisting of an injection device, an implant, or a biodegradable container.

In an eighth aspect of the invention, the invention relates to a device, such as an injection device, an implant, or a biodegradable container, comprising the peptide or pharmaceutically acceptable salt thereof according to he first aspect, the pharmaceutical composition according to the second aspect of the invention, or the cosmetic composition according to the fifth aspect of the invention.

### BRIEF DESCRIPTION OF THE DRAWINGS

FIG. 1 shows the peptide synthesis flow.

FIG. 2. Human hepatoblastoma cell line HepG2 was treated with vehicle (DMSO) in the absence or presence of 30 mM fructose and Telmisartan (5 μg/ml), Semaglutide (10 nM) or GP6 (10 or 40 μg/ml) for 5 days. RNA was extracted and mRNA levels of Acetyl-CoA Carboxylase (ACC) (panel A) and Peroxisome proliferator-activated receptor gamma (PPARγ) (panel B) relative to 18S were measured by RTPCR.

FIG. 3 Human hepatic stellate cell line LX-2 was treated with vehicle (DMSO) or TGFβ1 (10 ng/ml) in the absence or presence of GP6 (10 or 40 μg/ml) for 24 h. RNA was extracted and mRNA levels of collagen 1A1 (Col1A1) relative to 18S were measured by RTPCR.

FIG. 4 Mature human subcutaneous adipocytes were treated with vehicle (DMSO), 1 μM of Isoproterenol, Semaglutide (10 nM) or GP6 (25, 50 or 100 μg/ml) for 16 h. Glycerol concentration was assessed using a Lipolysis Assay Kit (Tebu-bio).

FIG. 5 Human hepatoblastoma cell line HepG2 was treated with vehicle (DMSO) or 10 μM of GP4, GP7, GP8 or GP9 for 24 h. RNA was extracted and mRNA levels of Acetyl-CoA Carboxylase (ACC), Fatty Acid Synthase (FAS) and ATP-Citrate Lyase (ACLY) relative to 18S were measured by RTPCR.

FIG. 6. Human hepatic stellate cell line LX-2 was treated with vehicle (DMSO) or TGFβ1 (10 ng/ml) in the absence or presence of 8 μM of GP8 or GP9 for 24 h. RNA was extracted and mRNA levels of collagen 1A1 (Col1A1) relative to 18S were measured by RTPCR.

FIG. 7. Mature human subcutaneous adipocytes were treated with vehicle (DMSO), 3 μM of Isoproterenol or 10 μM of GP4, GP8 or GP9 for 16 h. Glycerol concentration was assessed using a Lipolysis Assay Kit (Tebu-bio). Lipolysis induction was calculated with respect to the control cells treated with DMSO.

FIG. 8. Total body weight (panel A) and body weight increment (panel B) were measured at the end of the experiment in vehicle and GP6 treated mice. Mpk: mg/kg; SC: subcutaneous administration; QD: once a day administration.

FIG. 9. Liver (panel A) and adipose tissue from subcutaneous (panel B), visceral (panel C) and brown (panel D) origin were weighed at the end of the experiment in vehicle and GP6 treated mice. Mpk: mg/kg; SC: subcutaneous administration; QD: once a day administration.

FIG. 10. H&E histological examination of livers from vehicle and GP6 treated mice at the end of the experiment. Mpk: mg/kg; SC: subcutaneous administration; QD: once a day administration.

FIG. 11. Liver triglyceride (TAG) content (panel A) of vehicle and GP6 treated mice at the end of the experiment. Brunt score (panel B), Steatosis (panel C) and % of steatosis (panel D) were analyzed in H&E histological sections. Mpk: mg/kg; SC: subcutaneous administration; QD: once a day administration.

FIG. 12. Peroxisome Proliferator Activator Receptor gamma (PPARY) (panel B), Acetyl-CoA Carboxylase (ACC) (panel A) and Fatty Acid Synthase (FAS) (panel C) mRNA levels relative to 18S were measured by RTPCR in livers from vehicle and GP6 treated mice. Mpk: mg/kg; SC: subcutaneous administration; QD: once a day administration.

FIG. 13. F4/80 (panel D), TNFα (panel A), IL1β (panel B) and IL6 (panel C) mRNA levels relative to 18S were measured by RTPCR in livers from vehicle and GP6 treated mice. Mpk: mg/kg; SC: subcutaneous administration; QD: once a day administration.

FIG. 14. F4/80 mRNA levels relative to 18S were measured by RTPCR in adipose tissue of control, vehicle and GP6 treated mice. Mpk: mg/kg; SC: subcutaneous administration; QD: once a day administration.

FIG. 15. Col1A1 mRNA levels relative to 18S were measured by RTPCR in adipose tissue of control, vehicle and GP6 treated mice. Mpk: mg/kg; SC: subcutaneous administration; QD: once a day administration.

FIG. 16. Body weight increment (panel A) and liver weight (panel B) were measured at the end of the experiment in vehicle and GP6 treated mice. Mpk: mg/kg; SC: subcutaneous administration; QD: once a day administration.

FIG. 17. AST (panel A) and ALT (panel B) activity levels in serum were measured at the end of the experiment in vehicle and GP6 treated mice. Mpk: mg/kg; SC: subcutaneous administration; QD: once a day administration.

FIG. 18. Picrosirius staining of livers from vehicle and GP6 treated mice at the end of the experiment (panel A), collagen staining pointed by black arrows. Picrosirius red quantification using ImageJ software of all sections (Panel B). Fibrosis stage determination by the pathologist of all sections. Mpk: mg/kg; SC: subcutaneous administration; QD: once a day administration.

FIG. 19. Col1A1 mRNA levels relative to 18S were measured by RTPCR in livers of vehicle and GP6 treated mice. Mpk: mg/kg; SC: subcutaneous administration; QD: once a day administration.

FIG. 20. F4/80 mRNA levels relative to 18S were measured by RTPCR in livers of vehicle and GP6 treated mice. Mpk: mg/kg; SC: subcutaneous administration; QD: once a day administration.

FIG. 21. Total body weight (panel A) and liver weight (panel B) were measured at the end of the experiment in control, vehicle, Telmisartan and GP6 treated mice. Mpk: mg/kg; SC: subcutaneous administration; QD: once a day administration.

FIG. 22. Glucose and AST activity levels in serum were measured at the end of the experiment in control, vehicle, Telmisartan and GP6 treated mice. Mpk: mg/kg; SC: subcutaneous administration; QD: once a day administration.

FIG. 23. H&E histological examination of livers from control, vehicle and GP6 treated mice at the end of the experiment. Mpk: mg/kg; SC: subcutaneous administration; QD: once a day administration.

FIG. 24. Liver triglyceride (TAG) content (panel A) of control, vehicle, Telmisartan and GP6 treated mice at the end of the experiment. Steatosis (panel C) and Brunt score (panel B) analysis were performed in H&E histological sections. Mpk: mg/kg; SC: subcutaneous administration; QD: once a day administration.

FIG. 25. Peroxisome Proliferator Activator Receptor gamma (PPARY) (panel A) and Acetyl-CoA Carboxylase (ACC) (panel B) mRNA levels relative to 18S were measured by RTPCR in livers of control, vehicle and GP6 treated mice. Mpk: mg/kg; SC: subcutaneous administration; QD: once a day administration.

FIG. 26. Picrosirius staining of livers of control, vehicle and GP6 treated mice at the end of the experiment. White arrows show normal collagen deposits and black arrows show collagen deposits from increase hepatic fibrosis. Mpk: mg/kg; SC: subcutaneous administration; QD: once a day administration.

FIG. 27. Col1A1 (panel A) and TGFβ1 (panel B) mRNA levels relative to 18S were measured by RTPCR in livers of control, vehicle and GP6 treated mice. Mpk: mg/kg; SC: subcutaneous administration; QD: once a day administration.

FIG. 28. F4/80 (panel A) and TNFα mRNA levels relative to 18S were measured by RTPCR in livers of control, vehicle and GP6 treated mice. Mpk: mg/kg; SC: subcutaneous administration; QD: once a day administration.

FIG. 29. F4/80 (panel A) and IL1β (Panel B) mRNA levels relative to 18S were measured by RTPCR in livers of control, vehicle and GP6 treated mice. Mpk: mg/kg; SC: subcutaneous administration; QD: once a day administration.

FIG. 30. Col1A1 mRNA levels relative to 18S were measured by RTPCR in adipose tissue of control, vehicle and GP6 treated mice. Mpk: mg/kg; SC: subcutaneous administration; QD: once a day administration.

FIG. 31. Col1A1 mRNA levels relative to 18S were measured by RTPCR in heart of control, vehicle and GP6 treated mice. Mpk: mg/kg; SC: subcutaneous administration; QD: once a day administration.

FIG. 32. Effect of GP6 and modified peptides on hepatic lipogenesis in HepG2 cells. Each bar within each group represents mRNA levels of a key hepatic lipogenic enzyme, from left to right: Acetyl-CoA Carboxylase (ACC), ATP Citrate Lyase (ACLY) and Fatty Acid Synthase (FAS), and a key transcription factor regulating several lipogenic enzymes such as PPARγ (PPARγ).

FIG. 33. Effect of different peptides and SHBG on fibrosis in LX-2 cells.

FIG. 34. Effects of different peptides and SHBG in reducing hepatic lipogenic key enzymes in the presence of 30 mM fructose. Each bar within each group represents mRNA levels of a key hepatic lipogenic enzyme, from left to right: Acetyl-CoA Carboxylase (ACC), ATP Citrate Lyase (ACLY) and Fatty Acid Synthase (FAS), and a key transcription factor regulating several lipogenic enzymes such as PPARγ (PPARγ).

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

For the purposes of the present invention, any ranges given include both the lower and the upper end-points of the range.

### I—Peptide of the Invention

In a first aspect, the invention relates to a peptide or a pharmaceutically acceptable salt thereof, wherein the peptide has a length of 9 to 49 amino acids, particularly of 10-40 amino acids, each one of the amino acids being L- or D-amino acid, and that comprises peptide fragment F1, and additionally fragment/s F2 and/or F3 as defined above. In one embodiment, the peptide of the first aspect has a length of 9 to 49 amino acids, particularly of 10-40 amino acids, each one of the amino acids being L- or D-amino acid, and consists of peptide fragment F1, and additionally fragment/s F2 and/or F3 as defined above. More particularly, the peptide of the first aspect has a length of 10-40 amino acids, and:
i) peptide fragment F1 has SEQ ID NO. 9: TWDPEGVIFY or is a variant thereof that has at least 80% sequence identity with SEQ ID NO.9, wherein each one of the amino acids of peptide fragment F1 is a L- or D-amino acid;
ii) peptide fragment F2 has SEQ ID NO. 13 (TKTSSSFEVR) or is a variant thereof with at least 80% sequence identity with SEQ ID NO.13; or has SEQ ID NO. 14 (VMTFDLTKITKTSSSFEVR) or is a variant thereof with at least 80% sequence identity with SEQ ID NO.14; and wherein each one of the amino acids of peptide fragment F2 is a L- or D-amino acid;
and
iii) peptide fragment F3 has SEQ ID NO. 4 (GDTNPKDDWF) or is a variant thereof with at least 80% sequence identity with SEQ ID NO.4; or has SEQ ID NO. 15 (GDTNPKDDWFMLGLRDGRPE) or is a variant thereof with at least 80% sequence identity with SEQ ID NO.15; and wherein each one of the amino acids of peptide fragment F3 is a L- or D-amino acid.

The term “peptide”, or “polypeptide”, as used herein, refers to the term commonly known by a skilled person. In particular, the term refers to short chains of amino acids, typically between 2 and 50 amino acids in length, linked by peptide bonds. Peptide bonds linking the amino acids of a peptide generally form through a dehydration reaction and join the carboxyl group of one amino acid to the amine group of the next amino acid in a head-to-tail manner to form a peptide chain. This leaves a free carboxylic group at one end of the peptide, called the C-terminus, and a free amine group on the other end called the N-terminus. When the protein is translated from messenger RNA, it is created from N-terminus to C-terminus. The convention is to write the amino acid sequence of the peptide from N- to C-terminus, and thus to put the C-terminal end on the right.

In an embodiment of the first aspect of the invention, fragment F2 is directly linked to the N-terminus of fragment F1. In another embodiment, the peptide of the invention does not comprise fragment F3, and fragment F2 is directly linked to the N-terminus of fragment F1. In another embodiment, the peptide of the invention consists of fragment F2 and fragment F1 and fragment F2 is directly linked to the N-terminus of fragment F1.

In another embodiment of the first aspect of the invention, fragment F3 is directly linked to the C-terminus of fragment F1. In another embodiment, the peptide of the invention does not comprise fragment F2 and fragment F3 is directly linked to the C-terminus of fragment F1. In another embodiment, the peptide of the invention consists of fragments F1 and fragment F3 and fragment F3 is directly linked to the C-terminus of fragment F1.

In another embodiment of the first aspect of the invention, the peptide of the invention comprises fragments F2, F1 and F3 and fragment F2 is directly linked to the N-terminus of fragment F1 and fragment F3 is directly linked to the C-terminus of fragment F1. In another embodiment of the peptide of the invention, the peptide consists of fragments F2, F1 and F3 and fragment F2 is directly linked to the N-terminus of fragment F1 and fragment F3 is directly linked to the C-terminus of fragment F1.

As well understood by a skilled person, the term “directly linked”, as used herein, refers to the fact that the C-terminal amino acid of a peptide fragment (i.e. the last amino acid of the amino acid chain of the peptide) is linked to the N-terminal amino acid of another peptide fragment (the first amino acid of the amino acid chain of said peptide) by a peptide bond. Thus, in a particular embodiment, the term “directly linked” in the context of two peptide fragments refers to the fact that the two fragments are directly linked by a peptide bond. In a more particular embodiment, the term “directly linked” in the context of two peptide fragments refers to the fact that the C-terminal amino acid of one fragment is linked to the N-terminal amino acid of the other fragment by a peptide bond.

In a particular embodiment of the first aspect, peptide fragment F2 is linked to the N-terminus of peptide fragment F1 by a peptide bond, particularly by a peptide bond between the C-terminal amino acid of fragment F2 and the N-terminal amino acid of fragment F1. In another particular embodiment, peptide fragment F3 is linked to the C-terminus of peptide fragment F1 by a peptide bond, particularly by a peptide bond between the C-terminal amino acid of fragment F1 and the N-terminal amino acid of fragment F3.

The expression “length of a peptide”, or “length of a fragment peptide”, as used herein, refers to the number of amino acids forming said peptide or peptide fragment. As well understood by a skilled person, a peptide with a length of 4-10 amino acids is peptide that has 4-10 amino acids, and a peptide with a length of 15 amino acids is a peptide that has 15 amino acids or whose number of amino acids is 15. Therefore, the expression “the peptide has a length of 9 to 49 amino acids” or “the peptide has 9 to 49 amino acids”, or a “peptide with a length of 9-49 amino acids” as used herein, refers to a peptide whose number of amino acids is of 9-49.

In an embodiment, the peptide of the invention has a length of 9-49, 10-49, 11-49, 12-49, 13-49, 14-49, 15-49, 16-49, 17-49, 18-49, 18-49, 19-49 or 20-49 amino acids, particularly 14-49. In a particular embodiment, the length of the peptide of the invention is of 15-49. In another particular embodiment, the length of the peptide of the invention is of 20-49. In another particular embodiment, the length of the peptide of the inventions is of 10-48, 11-48, 12-48, 13-48, 14-48, 15-48, 16-48, 17-48, 18-48, 18-48, 19-48 or 20-48 amino acids, more particularly, 10-48 amino acids. In another embodiment, the peptide of the invention has a length of 9-49, 9-48, 9-47, 9-46, 9-45, 9-44, 9-43, 9-42, 9-41, 9-40, 9-39-, 9-38, 9-37, 9-36, 9-35, 9-34, 9-33, 9-32, 9-31, 9-30, 9-29, 9-28, 9-27, 9-26, 9-25, 9-24, 9-23, 9-22, 9-21, 9-20 amino acids, particularly 9-30 amino acids. In another embodiment, the peptide of the invention has a length of 10-48, 11-45, 12-40, 12-35, 15-35, 15-30 amino acids, particularly 15-30 amino acids. In another embodiment, the peptide of the invention has a length of 20-30 amino acids. In another embodiment, the peptide of the invention has a length of 15-20 amino acids. In a more particularly embodiment, the peptide of the invention has a length of 12-35, even more particularly 15-30 amino acids. In another embodiment, the length of the peptide of the invention is selected from the list consisting of 15, 16, 17, 18, 19, 20, 28 and 30 amino acids. In another embodiment, the length of the peptide of the invention is selected from the list consisting of 20, 28, 29 and 30 amino acids.

In another more particular embodiment, the length of the peptide of the invention is selected from the list consisting of 20, 29 and 30 amino acids. In another particular embodiment, the peptide of the invention has a length of 20 amino acids. In another particular embodiment, the peptide of the invention has a length of 28 amino acids. In another particular embodiment, the peptide of the invention has a length of 29 amino acids. In another particular embodiment, the peptide of the invention has a length of 30 amino acids.

The term “peptide fragment” or “fragment” as used herein refers to a peptide subunit of the corresponding peptide. Thus, said fragment is a short chain of amino acids comprised within a peptide and with a length shorter than that of said peptide, i.e. with a number of amino acids lower than the number of amino acids of said peptide.

In the context of the peptide of the invention, the peptide fragments referred to as F1, F2 and F3 herein are amino acid chains that have a length shorter than that of the peptide of the invention. Thus, in an embodiment, the length of the peptide of the invention is equal or higher than the sum of the lengths of the fragments comprised in the peptide of the invention and that are defined as F1, F2 and F3. As well understood by a skilled person, if the length of the peptide of the invention is higher than the sum of the lengths of said fragments, the peptide of the invention comprises, in addition to said fragments, additional amino acids. In a more particular embodiment, the length of the peptide of the invention is equal to the sum of the lengths of the fragments comprised in the peptide of the invention and that are defined as F1, F2 and F3. In a particular embodiment, the length of the peptide of the invention is equal or higher than the sum of the lengths of the fragments defined as F1, F2, and F3 which are comprised in the peptide of the invention.

In a particular embodiment, the length of the peptide of the invention is equal to the sum of the lengths of the fragments defined as F1, F2, and F3 which are comprised in the peptide of the invention.

In an embodiment of the first aspect of the invention, peptide fragment F1 has a length of 4-10, 5-10, 6-10, 7-10, 8-10, or 9-10 amino acids. In a particular embodiment, fragment F1 has a length of 5-10. In a more particular embodiment, fragment F1 has a length of 10 amino acids.

In another embodiment of the first aspect of the invention, peptide fragment F2 has a length of 5-19, 6-19, 7-19, 8-19, 9-19, 10-19, 10-19, 11-19, 12-19, 13-19, 14-19, 15-19, 16-19, 17-19, or 18-19 amino acids. In a particular embodiment, the peptide fragment F2 has a length of 19 amino acids. In another embodiment, the peptide fragment F2 has a length of 5-10, 6-10, 7-10, 8-10, or 9-10 amino acids. In a particular embodiment, peptide fragment F2 has a length of 5, 6, 7, 8, 9 or 10 amino acids, more particularly 10 amino acids.

In another embodiment of the peptide of the invention, peptide fragment F3 has a length of 10-20, 11-20, 12-20, 13-20, 14-20, 15-20, 16-20, 17-20, 18-20, or 19-20 amino acids. In a particular embodiment, fragment F3 has a length of 20 amino acids. In a more particular embodiment, fragment F3 has a length of 10 amino acids.

The term “basic amino acid”, or “basic and polar amino acid”, as used herein, refers to amino acids with side chains containing nitrogen and resembling ammonia which is a base (such as amines, guanidines, or imidazole). Non-limitative examples of basic amino acids are Lys, Arg, and His. In a particular embodiment, the basic amino acids referred herein are Lys, Arg, and/or His.

The term “Hydrophobic amino acids”, as used herein, refers to amino acids having hydrophobic side chains, which tend to be repelled from water. When part of a protein, these amino acids typically form the hydrophobic core of proteins, which is isolated from the polar solvent. Non-limitative examples of hydrophobic amino acids include glycine (Gly), alanine (Ala), valine (Val), leucine (Leu), isoleucine (Ile), proline (Pro), phenylalanine (Phe), methionine (Met), and tryptophan (Trp). In a particular embodiment, the hydrophobic amino acids referred herein are selected from the list consisting of glycine (Gly), alanine (Ala), valine (Val), leucine (Leu), isoleucine (Ile), proline (Pro), phenylalanine (Phe), methionine (Met), and tryptophan (Trp). In another embodiment, the hydrophobic amino acids referred herein are selected from the list consisting of alanine (Ala), valine (Val), leucine (Leu), isoleucine (Ile), proline (Pro), phenylalanine (Phe) and cysteine (Cys). In a particular embodiment, the hydrophobic amino acids referred herein are selected from the list consisting of alanine (Ala), valine (Val), and leucine (Leu).

As used herein, the term “pharmaceutically acceptable salt”, or “acceptable salt” or “salt” refers to those salts which are, within the scope of sound medical judgment, suitable for use in contact with the tissues of humans and lower animals without undue toxicity, irritation, allergic response and the like, and are commensurate with a reasonable benefit/risk ratio. Appropriate acceptable salts are well known in the art. Examples of pharmaceutical acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other acceptable salts include adipate, alginate, ascorbate, aspartate, benzenesulfonate, benzoate, bisulphate, borate, butyrate, camphorate, camphorsulfonate, citrate, cyclopentanepropionate, digluconate, dodecylsulphate, ethanesulfonate, formate, fumarate, glucoheptonate, glycerophosphate, gluconate, hemisulphate, heptanoate, hexanoate, hydroiodide, 2-hydroxy-ethanesulfonate, lactobionate, lactate, laurate, lauryl sulphate, malate, maleate, malonate, methanesulfonate, 2-naphthalenesulfonate, nicotinate, nitrate, oleate, oxalate, palmitate, pamoate, pectinate, persulphate, 3-phenylpropionate, phosphate, picrate, pivalate, propionate, stearate, succinate, sulphate, tartrate, thiocyanate, p-toluenesulfonate, undecanoate, valerate salts, and the like. Salts derived from appropriate bases include alkali metal, alkaline earth metal, and ammonium. Representative alkali or alkaline earth metal salts include sodium, lithium, potassium, calcium, magnesium, and the like. Further acceptable salts include, when appropriate, nontoxic ammonium, quaternary ammonium, and amine cations formed using counterions such as halide, hydroxide, carboxylate, sulphate, phosphate, nitrate, lower alkyl sulfonate and aryl sulfonate.

In an embodiment, fragment F1 of the peptide of the invention has a length of 4-10, 5-10, 6-10, 7-10, 8-10, 9-10, or 10 amino acids, more particularly, 4-10 amino acids, each one of the amino acids being L- or D-amino acid, and has a sequence SEQ ID NO. 5: (X¹)ₐ(X²)_{b}D (X⁴)E G(X⁶)_{d}(X⁷)ₑ(X⁸)_{f}(Y)_{g},
wherein:
X¹ represents T, or A,
X² represents W, N, Q, K or A;
X⁴ represents P, N or L,
X⁶ and X⁷ are the same or different and represent A, V, L or I;
X⁸ represents F or P,
subindexes “a”, “b”, “d”, “e”, “f” and “g”” are integer numbers independently selected from 0 and 1.

In another embodiment, fragment F1 of the peptide of the invention has a length of 4-10, 5-10, 6-10, 7-10, 8-10, 9-10, or 10 amino acids, more particularly, 4-10 amino acids, each one of the amino acids being L- or D-amino acid, and has a sequence SEQ ID NO. 6: (X¹)ₐ(X²)_{b}D P E G(X⁶)_{d}(X⁷)ₑ(X⁸)_{f}(Y)_{g}
wherein:
X¹ represents T, or A,
X² represents W, N, Q, K or A,
X⁶ and X⁷ are the same or different and represent A, V, L or I,
X⁸ represents F or P,
subindexes “a”, “b”, “d”, “e”, “f” and “g” are integer numbers independently selected from 0 and 1;

In another embodiment, fragment F1 of the peptide of the invention has a length of 4-10, 5-10, 6-10, 7-10, 8-10, 9-10, or 10 amino acids, more particularly, 4-10 amino acids, each one of the amino acids being L- or D-amino acid, and has a sequence SEQ ID NO. 7: (X¹)ₐ(X²)_{b}D L E G(X⁶)_{d}(X⁷)ₑ(X⁸)_{f}(Y)_{g},
wherein:
X¹ represents T, or A,
X² represents W, N, Q, K or A;
X⁶ and X⁷ are the same or different and represent A, V, L or I;
X⁸ represents F or P,
subindexes “a”, “b”, “d”, “e”, “f” and “g” are integer numbers independently selected from 0 and 1.

In another embodiment, peptide fragment F1 of the peptide of the invention has a length of 4-10, 5-10, 6-10, 7-10, 8-10, 9-10, or 10 amino acids, more particularly, 4-10 amino acids, each one of the amino acids being L- or D-amino acid, and has a sequence SEQ ID NO. 8: (X¹)ₐ(X²)_{b}D N E G(X⁶)_{d}(X⁷)ₑ(X⁸)_{f}(Y)_{g},
wherein:
X¹ represents T, or A,
X² represents W, N, Q K or A;
X⁶ and X⁷ are the same or different and represent A, V, L or I;
X⁸ represents F or P,
subindexes “a”, “b”, “d”, “e”, “f” and “g” are integer numbers independently selected from 0 and 1.

In another embodiment, the peptide fragment F1 of the peptide of the invention has a sequence selected from the group consisting of SEQ ID NO. 78-82.

In another embodiment, the peptide fragment F1 of the peptide of the invention has a length of 5-9, 6-9, 7-9, 8-9, or 9 amino acids, particularly 5-9 amino acids, each one of the amino acids being L- or D-amino acid and has sequence SEQ ID NO.10: (T)ₐ(X²)_{b}D(X⁴)_{c}EG(X⁶)_{d}(X⁷)ₑ(X⁸)_{f}(Y)_{g}
wherein:
X² represents W, N, Q or K
X⁴ represents P, N, a basic amino acid or a hydrophobic amino acid, preferably P, N, L, V, or A,
X⁶ and X⁷ are the same or different and represent A, V, L or I,
X⁸ represents F or P,
subindexes “a”, “b”, “c”, “d” “e”, “f” and “g” are integer numbers independently selected from 0 and 1; provided that two, three, four, five or six of the subindexes “a”, “b”, “c” “d”, “e”, “f” and “g” represent 1; and the remaining subindexes represent zero.

In a particular embodiment, the peptide fragment F1 of the peptide of the invention has a length of 5-9, 6-9, 7-9, 8-9, or 9 amino acids, particularly 5-9 amino acids, each one of the amino acids being L- or D-amino acid, and has SEQ ID NO. 10, wherein:
X² represents N,
X⁴ represents P, L, V, or A,
X⁶ and X⁷ are the same or different and represent L, V, or A, and/or
X⁸ represents F or P.

In another embodiment the peptide fragment F1 of the peptide of the invention has a length of 5-9, 6-9, 7-9, 8-9, or 9 amino acids, particularly 5-9 amino acids, each one of the amino acids being L- or D-amino acid, and has SEQ ID NO. 83: (T)ₐ(X²)_{b}D(X⁴)EG(X⁶)_{d}(X⁷)ₑ(X⁸)_{f}(Y)_{g}, wherein a, b, d, e, f, g, X², X⁶, X⁷ and X⁸ are as defined in SEQ ID NO. 10 and X⁴ represents P, L or N.

In another embodiment the peptide fragment F1 of the peptide of the invention has a length of 5-9, 6-9, 7-9, 8-9, or 9 amino acids, particularly 5-9 amino acids, each one of the amino acids being L- or D-amino acid, and has SEQ ID NO. 84: (T)ₐ(X²)_{b}DPEG(X⁶)_{d}(X⁷)ₑ(X⁸)(Y)_{g}, wherein a, b, d, e, f, g, X², X⁶, X⁷ and X⁸ are as defined in SEQ ID NO. 10.

In another embodiment, the peptide fragment F1 of the peptide of the invention has a length of 5-9, 6-9, 7-9, 8-9, or 9 amino acids, particularly 5-9 amino acids, each one of the amino acids being L- or D-amino acid, and has a SEQ ID NO. 85: (T)ₐ(X²)_{b}DLEG(X⁶)_{d}(X⁷)ₑ(X⁸)(Y)_{g}, wherein a, b, d, e, f, g, X², X⁶, X⁷ and X⁸ are as defined in SEQ ID NO. 10.

In another embodiment the peptide fragment F1 of the peptide of the invention has a length of 5-9, 6-9, 7-9, 8-9, or 9 amino acids, particularly 5-9 amino acids, each one of the amino acids being L- or D-amino acid, and has SEQ ID NO. 86: (T)ₐ(X²)_{b}DNEG(X⁶)_{d}(X⁷)ₑ(X⁸)(Y)_{g}, wherein a, b, d, e, f, g, X², X⁶, X⁷ and X⁸ are as defined in SEQ ID NO. 10.

In another embodiment, fragment F1 of the peptide of the invention has a sequence selected from the group consisting of SEQ ID NO. 67-76 In another embodiment, the peptide fragment F1 of the peptide of the invention has a length of 5-9, 6-9, 7-9, 8-9, or 9 amino acids, particularly 5-9 amino acids, each one of the amino acids being L- or D-amino acid and has sequence SEQ ID NO.11: (T)ₐ(X²)_{b}D(X⁴)EG(X⁶)_{d}(X⁷)ₑ(F)_{f}(Y)_{g}
wherein:
X² represents W, N, Q or K,
X⁴ represents P, N or a basic amino acid,
X⁶ and X⁷ are the same or different and represent A, V, L or I,
subindexes “a”, “b”, “d”, “e”, “f”, and “g” are integer numbers independently selected from 0 and 1; provided that one, two, three, four or five of the subindexes “a”, “b”, “d” “e”, “f”, and “g” represent 1; and the remaining subindexes represent zero.

In a particular embodiment the peptide fragment F1 is as defined in the embodiment above, wherein:
X² represents W or N,
X⁴ represents P or N, and/or
X⁶ or X⁷ represents V, L or I.

In another embodiment the peptide fragment F1 of the peptide of the invention has a length of 5-9, 6-9, 7-9, 8-9, or 9 amino acids, particularly 5-9 amino acids, each one of the amino acids being L- or D-amino acid, and has a SEQ ID NO. 87: (T)ₐ(X²)_{b}D(X⁴)EG(X⁶)_{d}(X⁷)ₑ(F)_{f}(Y)_{g}, wherein a, b, d, e, f, g, X², X⁶ and X⁷ are as defined in SEQ ID NO. 11 and X⁴ represents P, L or N.

In another embodiment the peptide fragment F1 of the peptide of the invention has a length of 5-9, 6-9, 7-9, 8-9, or 9 amino acids, particularly 5-9 amino acids, each one of the amino acids being L- or D-amino acid, and has SEQ ID NO. 88: (T)ₐ(X²)_{b}DPEG(X⁶)_{d}(X⁷)ₑ(F)(Y)_{g}, wherein a, b, d, e, f, g, X², X⁶ and X⁷ are as defined in SEQ ID NO. 11.

In another embodiment, the peptide fragment F1 of the peptide of the invention has a length of 5-9, 6-9, 7-9, 8-9, or 9 amino acids, particularly 5-9 amino acids, each one of the amino acids being L- or D-amino acid, and has a SEQ ID NO. 89: (T)ₐ(X²)_{b}DNEG(X⁶)_{d}(X⁷)ₑ(F)_{f}(Y)_{g}, wherein a, b, d, e, f, g, X², X⁶, X⁷ and X⁸ are as defined in SEQ ID NO. 11.

In another embodiment, the peptide fragment F1 of the peptide of the invention has a length of 5-9, 6-9, 7-9, 8-9, or 9 amino acids, particularly 5-9 amino acids, each one of the amino acids being L- or D-amino acid, and has a SEQ ID NO. 90: (T)ₐ(X²)_{b}DLEG(X⁶)_{d}(X⁷)ₑ(F)(Y)_{g}, wherein a, b, d, e, f, g, X², X⁶, X⁷ and X⁸ are as defined in SEQ ID NO. 11.

In another embodiment the peptide fragment F1 of the peptide of the invention has a sequence selected from the group consisting of: SEQ ID NO. 23-63.

In another embodiment, the peptide of the invention wherein fragment F1 has a sequence selected from the group consisting of SEQ ID NO. 10, 11, 83-90, 23-63, 67-76, has a length of 10-48, 11-48, 12-48, 13-48, 14-48, 15-48, 16-48, 17-48, 18-48, 18-48, 19-48, 20-48 amino acids, more particularly, 10-48 amino acids.

In a particular embodiment, peptide fragment F1 has SEQ ID NO. 9: TWDPEGVIFY or is a sequence variant thereof that has at least 70% sequence identity with SEQ ID NO.9, wherein each one of the amino acids of peptide fragment F1 is a L- or D-amino acid. In another particular embodiment, peptide fragment F1 is a sequence variant of SEQ ID NO. 9 that has at least 80% sequence identity with SEQ ID NO.9, wherein each one of the amino acids of peptide fragment F1 is a L- or D-amino acid. In another particular embodiment, peptide fragment F1 is a sequence variant of SEQ ID NO. 9 that has at least 90% sequence identity with SEQ ID NO.9, wherein each one of the amino acids of peptide fragment F1 is a L- or D-amino acid.

The term “sequence variant” is defined below. In a particular embodiment, the sequence variant thereof, in the context of variants of a peptide fragment with a specific sequence, substantially maintains or improves the activities of the reference peptide fragment from which it derives in the peptide of the invention. Thus, in another particular embodiment, the peptide of the invention that comprises a sequence variant of a peptide fragment, substantially maintains or improves the activity of the peptide reference, wherein the peptide reference has the same sequence as the former, except that it comprises the reference peptide fragment, instead of the sequence variant thereof.

Thus, in a particular embodiment, the peptide fragment F1 referred in any of the embodiment herein that is a variant of SEQ ID NO. 9, substantially maintains or improves the activities of peptide fragment F1 with SEQ ID NO.9 in the peptide of the invention. Thus, in another particular embodiment, the peptide of the invention wherein fragment F1 is a sequence variant of SEQ ID NO.9, substantially maintains or improves the activity of the same peptide comprising instead of F1 as a sequence variant of SEQ ID NO.9, F1 with SEQ ID NO.9.

As used herein, by “substantially maintains or improves the activity of the reference peptide fragment” is meant that the sequence variant of said peptide fragment has at least 90%, more particularly at least 95%, or even more particularly at least 99% of the activity of the reference peptide fragment in the peptide of the invention or when part of the peptide of the invention. Thus, in another particular embodiment, the peptide of the invention that comprises a sequence variant of a peptide fragment, has at least 90%, more particularly at least 95%, or even more particularly at least 99% of the activity of the peptide reference, wherein the peptide reference has the same sequence as the former, except that it comprises the reference peptide fragment, instead of the sequence variant thereof.

In another particular embodiment, the activity of the peptide fragment or of the peptide of the invention, as used herein, refers to the lipolytic, anti-lipogenic, antifibrotic and/or anti-inflammatory activities of the peptide fragment or peptide, respectively. In a particular embodiment, it refers to the anti-lipogenic activity. In another particular embodiment, it refers to the anti-lipogenic and antifibrotic activities. In another particular embodiment, it refers to the anti-lipogenic and lipolytic activities. In another particular embodiment, it refers to the anti-lipogenic and anti-inflammatory activities. In another particular embodiment, it refers to the anti-lipogenic, antifibrotic and lipolytic activities. In another particular embodiment, it refers to the anti-lipogenic, antifibrotic and anti-inflammatory activities. In another particular embodiment, it refers to the anti-lipogenic, lipolytic and anti-inflammatory activities. In another particular embodiment, it refers to the anti-lipogenic, antifibrotic, lipolytic and anti-inflammatory activities. In another particular embodiment, it refers to the antifibrotic activity. In another particular embodiment, it refers to the antifibrotic and lipolytic. In another particular embodiment, it refers to the antifibrotic and anti-inflammatory activities. In another particular embodiment, it refers to the antifibrotic, lipolytic and anti-inflammatory activities. In another particular embodiment, it refers to the lipolytic activity. In another particular embodiment, it refers to the anti-inflammatory activity. In another particular embodiment, it refers to the lipolytic and anti-inflammatory activities. Particularly, it refers to the anti-lipogenic or antifibrotic activity. More particularly, it refers to the anti-lipogenic and antifibrotic activity.

Methods to determine the activity of the peptide of the invention, in particular the lipolytic, anti-lipogenic, antifibrotic and anti-inflammatory activities of a peptide, are provided below and apply herein. As well understood by a skilled person, methods to determine if a peptide fragment of interest maintains the activity of the reference peptide fragment in the peptide of the invention, comprise comparing the corresponding activity of the peptide of the invention comprising the peptide fragment of interest, with that of the same peptide, comprising instead of the peptide fragment of interest, the reference peptide fragment. In a more particular embodiment, peptide fragment F1 of the peptide of the invention has sequence SEQ ID NO. 9: TWDPEGVIFY, wherein each one of the amino acids is L- or D-amino acid. In another particular embodiment, the peptide of the invention comprising SEQ ID NO. 9 as fragment F1, has a length of 14-48 amino acids.

In an embodiment of the peptide of the invention, the peptide fragment F2 has a length of 5 to 19 amino acids, each one of the amino acids being L- or D-amino acid, and comprises sequence SEQ ID NO.2 (SF(X⁹)VR), wherein X⁹ represents E or Q. In a particular embodiment, the peptide fragment F2 consists of sequence SEQ ID NO.2 (SF(X⁹)VR), wherein X⁹ represents E or Q wherein each one of the amino acids is L- or D-amino acid.

In a particular embodiment of the peptide or pharmaceutically acceptable salt thereof of the invention, the peptide fragment F2 comprises sequence SEQ ID NO. 12 (SFEVR). In another particular embodiment of the peptide or pharmaceutically acceptable salt thereof of the invention, the peptide fragment F2 consists of sequence SEQ ID NO. 12 (SFEVR).

In a particular embodiment, the peptide fragment F2 of the peptide of the invention has SEQ ID NO. 13 (TKTSSSFEVR) or is a sequence variant thereof with at least 70% sequence identity with SEQ ID NO.13.

In another particular embodiment, the peptide fragment F2 is a sequence variant thereof with at least 80% sequence identity with SEQ ID NO.13. In another particular embodiment, the peptide fragment F2 is a sequence variant thereof with at least 90% sequence identity with SEQ ID NO.13.

In another particular embodiment, the peptide fragment F2 of the peptide of the invention has SEQ ID NO. 14 (VMTFDLTKITKTSSSFEVR) or is a sequence variant thereof with at least 70% sequence identity with SEQ ID NO.14. In another particular embodiment, the peptide fragment F2 is a sequence variant thereof with at least 80% sequence identity with SEQ ID NO.14. In another particular embodiment, the peptide fragment F2 is a sequence variant thereof with at least 90% sequence identity with SEQ ID NO.14.

The term “sequence variant of a peptide fragment” has been defined above in the context of fragment F1 and also applies herein. Thus, in a particular embodiment, the peptide fragment F2 referred in any of the embodiment herein that is a variant of SEQ ID NO. 13, substantially maintains or improves the activities of peptide fragment F2 with SEQ ID NO.13 in the peptide of the invention. Thus, in another particular embodiment, the peptide of the invention wherein fragment F2 is a sequence variant of SEQ ID NO.13, substantially maintains or improves the activity of the same peptide comprising instead of F1 as a sequence variant of SEQ ID NO.13, F1 with SEQ ID NO.13.

In another particular embodiment, the peptide fragment F2 referred in any of the embodiment herein that is a variant of SEQ ID NO. 14, substantially maintains or improves the activities of peptide fragment F2 with SEQ ID NO.14 in the peptide of the invention. Thus, in another particular embodiment, the peptide of the invention wherein fragment F2 is a sequence variant of SEQ ID NO.14, substantially maintains or improves the activity of the same peptide comprising instead of F2 as a sequence variant of SEQ ID NO.14, F2 with SEQ ID NO.14.

In an embodiment, the peptide fragment F2 of the peptide of the invention comprises SEQ ID NO. 13 (TKTSSSFEVR). In another embodiment, the peptide fragment F2 of the peptide of the invention consists of SEQ ID NO. 13 (TKTSSSFEVR). In another embodiment, the peptide fragment F2 of the peptide of the invention comprises SEQ ID NO. 14 (VMTFDLTKITKTSSSFEVR). In another embodiment, the peptide fragment F2 of the peptide of the invention consists of SEQ ID NO. 14 (VMTFDLTKITKTSSSFEVR).

In an embodiment of the peptide of the invention, the peptide fragment F3 has a length of 10 to 20 amino acids and comprises SEQ ID NO. 4 (GDTNPKDDWF).

In an embodiment, the peptide fragment F3 of the peptide of the invention comprises SEQ ID NO. 4 (GDTNPKDDWF). In an embodiment, the peptide fragment F3 of the peptide of the invention consists of SEQ ID NO. 4 (GDTNPKDDWF).

In a particular embodiment of the peptide of the invention, the peptide fragment F3 has SEQ ID NO. 4 (GDTNPKDDWF) or is a sequence variant thereof with at least 70% sequence identity with SEQ ID NO.4.

In another particular embodiment of the peptide of the invention, the peptide fragment F3 is a sequence variant thereof with at least 80% sequence identity with SEQ ID NO.4. In another particular embodiment of the peptide of the invention, the peptide fragment F3 is a sequence variant thereof with at least 90% sequence identity with SEQ ID NO.4.

In an embodiment, the peptide fragment F3 of the peptide of the invention comprises SEQ ID NO. 15 (GDTNPKDDWFMLGLRDGRPE). In another embodiment, the peptide fragment F3 of the peptide of the invention consists of SEQ ID NO. 15 (GDTNPKDDWFMLGLRDGRPE).

In a particular embodiment of the peptide of the invention, the peptide fragment F3 has SEQ ID NO. 15 (GDTNPKDDWF) or is a sequence variant thereof with at least 70% sequence identity with SEQ ID NO.15.

In another particular embodiment of the peptide of the invention, the peptide fragment F3 is a sequence variant thereof with at least 80% sequence identity with SEQ ID NO.15. In another particular embodiment of the peptide of the invention, the peptide fragment F3 is a sequence variant thereof with at least 90% sequence identity with SEQ ID NO.15.

The term “sequence variant of a peptide fragment” has been defined above in the context of fragment F1 and also applies herein. Thus, in a particular embodiment, the peptide fragment F3 referred in any of the embodiment herein that is a variant of SEQ ID NO. 4, substantially maintains or improves the activities of peptide fragment F3 with SEQ ID NO.4 in the peptide of the invention. Thus, in another particular embodiment, the peptide of the invention wherein fragment F3 is a sequence variant of SEQ ID NO.4, substantially maintains or improves the activity of the same peptide comprising instead of F3 as a sequence variant of SEQ ID NO.4, F3 with SEQ ID NO.4.

In another particular embodiment, the peptide fragment F3 referred in any of the embodiment herein that is a variant of SEQ ID NO. 15, substantially maintains or improves the activities of peptide fragment F3 with SEQ ID NO.15 in the peptide of the invention. Thus, in another particular embodiment, the peptide of the invention wherein fragment F3 is a sequence variant of SEQ ID NO.15, substantially maintains or improves the activity of the same peptide comprising instead of F3 as a sequence variant of SEQ ID NO.15, F3 with SEQ ID NO.15.

In an embodiment of the peptide of the invention, the peptide fragment F1 is SEQ ID NO. 9 (TWDPEGVIFY), the peptide fragment F2 is SEQ ID NO. 13 (TKTSSSFEVR) and/or the peptide fragment F3 is SEQ ID NO. 4 (GDTNPKDDWF). In a particular embodiment of the peptide of the invention, the peptide fragment F1 is SEQ ID NO. 9 (TWDPEGVIFY), the peptide fragment F2 is SEQ ID NO. 13 (TKTSSSFEVR) and the peptide fragment F3 is SEQ ID NO. 4 (GDTNPKDDWF). In a particular embodiment of the peptide of the invention, the peptide fragment F1 is SEQ ID NO. 9 (TWDPEGVIFY) and the peptide fragment F2 is SEQ ID NO. 13 (TKTSSSFEVR). In another particular embodiment of the peptide of the invention, the peptide fragment F1 is SEQ ID NO. 9 (TWDPEGVIFY) and the peptide fragment F3 is SEQ ID NO. 4 (GDTNPKDDWF).

In a particular embodiment of the peptide of the invention, the peptide of the inventions has sequence SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In a particular embodiment of the peptide of the invention, the peptide of the inventions has a length of 20-30 amino acids and sequence SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In more particular embodiment of the invention, the peptide of the invention has sequence SEQ ID NO. 16 (TKTSSSFEVRTWDPEGVIFY).

In a particular embodiment of the peptide of the invention, the peptide of the inventions has a sequence that is a variant of SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY), that is at least 70%, 75%, 85%, 85%, particularly at least 70% identical to SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In another particular embodiment, the peptide of the inventions has a sequence that is a sequence variant of SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY), and that is at least 70% identical to SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In another particular embodiment, the peptide of the inventions has a sequence that is a sequence variant of SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY), and that is at least 75% identical to SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In another particular embodiment, the peptide of the inventions has a sequence that is a sequence variant of SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY), and that is at least 80% identical to SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In another particular embodiment, the peptide of the inventions has a sequence that is a sequence variant of SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY), and that is at least 85% identical to SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In a more particular embodiment the peptide of the invention is a peptide with SEQ ID NO. 16 (TKTSSSFEVRTWDPEGVIFY) or a variant thereof at least 70%, 75%, 80%, 85%, particularly at least 90% identical to SEQ ID NO. 16 (TKTSSSFEVRTWDPEGVIFY).

In another particular embodiment, the peptide of the invention is a peptide with SEQ ID NO. 180 (ATKTSASFEVRTWDPEAVIFY), wherein each one of the amino acids is a L- or D-amino acid. In another particular embodiment, the peptide of the invention is a peptide with SEQ ID NO. 181 (TKTASAFEVRTWDPEGVIFYGDTNPKDDWF), wherein each one of the amino acids is a L- or D-amino acid.

In another particular embodiment, the peptide of the invention is a peptide with SEQ ID NO. 182 (TKTASAFEVRTWDPEGVIFY), wherein each one of the amino acids is a L- or D-amino acid.

In another particular embodiment, the peptide of the invention is a peptide with SEQ ID NO. 183 (TKTSASFEVRTWDPEGVIFYADTNPKDDWF), wherein each one of the amino acids is a L- or D-amino acid.

In a particular embodiment of the peptide of the invention, the peptide of the inventions has a sequence that is a variant of SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY), that is at least 90%, 92%, 95%, 97%, particularly at least 90% identical to SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In another particular embodiment, the peptide of the inventions has a sequence that is a sequence variant of SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY), and that is at least 92% identical to SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In another particular embodiment, the peptide of the inventions has a sequence that is a sequence variant of SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY), and that is at least 95% identical to SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In another particular embodiment, the peptide of the inventions has a sequence that is a sequence variant of SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY), and that is at least 97% identical to SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).

In a more particular embodiment the peptide of the invention is a peptide with SEQ ID NO. 16 (TKTSSSFEVRTWDPEGVIFY) or a variant thereof at least 90%, 92%, 95%, 97%, particularly at least 90% identical to SEQ ID NO. 16 (TKTSSSFEVRTWDPEGVIFY).

Peptide sequence variants are well understood to those of skill in the art and can involve amino acid sequence modifications. For example, amino acid sequence modifications typically fall into one or more of three classes: substitutional, insertional, or deletional variants. In a particular embodiment, the sequence modifications consist in the substitution of at least one amino acid or the reference amino acid sequence to an Alanine.

In the present invention the term “identical” or “identity” refers to the percentage of residues that are identical in the two sequences when the sequences are optimally aligned. If, in the optimal alignment, a position in a first sequence is occupied by the same amino acid residue as the corresponding position in the second sequence, the sequences exhibit identity with respect to that position. The percentage of identity determines the number of identical residues over a defined length in a given alignment. Thus, the level of identity between two sequences or (“percent sequence identity”) is measured as a ratio of the number of identical positions shared by the sequences with respect to the number of positions compared (i.e., percent sequence identity=(number of identical positions/total number of positions compared)×100). A gap, i.e., a position in an alignment where a residue is present in one sequence but not in the other, is regarded as a position with non-identical residues and is counted as a compared position.

A number of mathematical algorithms for rapidly obtaining the optimal alignment and calculating identity between two or more sequences are known and incorporated into a number of available software programs. For purposes of the present invention, the sequence identity between two amino acid sequences is preferably determined using algorithms based on global alignment, such as the Needleman-Wunsch algorithm (Needleman and Wunsch, 1970, J. Mol. Biol. 48: 443-453), preferably implemented in the Needle program of the EMBOSS package (EMBOSS: The European Molecular Biology Open Software Suite, Rice et al., 2000, Trends Genet. 16: 276-277); or the BLAST Global Alignment tool (Altschul et al., “Basic local alignment search tool”, 1990, J. Mol. Biol, v. 215, pages 403-410), using default settings. Local alignment also can be used when the sequences being compared are substantially the same length.

In a particular embodiment, the variant thereof substantially maintains or improves the lipolytic, anti-lipogenic, antifibrotic and anti-inflammatory activities of the reference peptide from which it derives. As used herein, by “substantially maintains or improves the lipolytic, anti-lipogenic, antifibrotic and anti-inflammatory activities” is meant that the variant thereof has at least 90%, more particularly at least 95%, or even more particularly at least 99% of the lipolytic, anti-lipogenic, antifibrotic and anti-inflammatory activities of the reference peptide. Methods to measure the lipolytic, anti-lipogenic, antifibrotic and anti-inflammatory of a peptide are provided herein below.

In an embodiment of the peptide of the invention, the peptide fragment F2 has a length of 5-10, 6-10, 7-10, 8-10, 9-10 or 10 amino acids, particularly 5-10 amino acids, each one of the amino acids being L- or D-amino acid and has sequence:

SEQ ID NO. 91:
(X¹)ₐ(X²)_{b}(X³)_{c}(X⁴)_{d}(X⁵)ₑSF(X⁶)VR

wherein:
X¹ represents T or A,
X² represents K or A,
X³ represents T or A,
X⁴ represents S or A,
X⁵ represents S or A,
X⁶ represents E or Q, and
subindexes a, b, c, d, and e are integer numbers independently selected from 0 and 1.

In another embodiment, fragment F2 has a sequence selected from the list consisting of: SEQ ID NO. 13, 92-106.

In another embodiment of the peptide of the invention, the peptide fragment F1 has a length of 7-10, 8-10, 9-10, or 10 amino acids, particularly 7-10 amino acids, each one of the amino acids being L- or D-amino acid and has sequence

SEQ ID NO. 108:
(X¹)ₐ(X²)(X³)(X⁴)_{c}(X⁵)G(V)_{d}(X⁷)(F)_{f}Y

wherein
X¹ represents T, or A,
X² represents W, N, or A,
X³ represents D or N,
X⁴ represents P, or L,
X⁵ represents E or Q,
X⁷ represents L or I, and
subindexes a, c, d and f are integer numbers independently selected from 0 and 1.

In another embodiment of the peptide of the invention, the peptide fragment F1 has a sequence selected from the group consisting of SEQ ID NO. 109-124, 107 and 179.

In a particular embodiment, the peptide of the invention has a length of 9-49, 12-35, 15-30, 15-20 amino acids, in particular 15-20 amino acids amino acids and comprises:
(i) a peptide fragment F1 with a length of 7-10, 8-10, 9-10, or 10 amino acids, each one of the amino acids being L- or D-amino acid and with sequence

SEQ ID NO. 108:
(X¹)a(X²)(X³)(X⁴)_{c}(X⁵)G(V)_{d}(X⁷)(F)_{f}Y

wherein
X¹ represents T, or A,
X² represents W, N, or A,
X³ represents D or N,
X⁴ represents P, or L,
X⁵ represents E or Q,
X⁷ represents L or I, and
subindexes a, c, d and f are integer numbers independently selected from 0 and 1, and
(ii) a peptide fragment F2 with a length of 5-10, 6-10, 7-10, 8-10, 9-10 or 10 amino acids amino acids, each one of the amino acids being L- or D-amino acid, and with sequence

SEQ ID NO. 91:
(X¹)ₐ(X²)_{b}(X³)_{c}(X⁴)_{d}(X⁵)ₑSF(X⁶)VR

wherein:
X¹ represents T or A,
X² represents K or A,
X³ represents T or A,
X⁴ represents S or A,
X⁵ represents S or A,
X⁶ represents E or Q, and
subindexes a, b, c, d, and e are integer numbers independently selected from 0 and 1.

In another embodiment, the peptide of the invention has a sequence selected from the group consisting of SEQ ID NO. 127-161.

In a particular embodiment of the peptide of the invention, peptide fragment F2 has a sequence selected from the group consisting of SEQ ID NO. 91, 13, 92-106, wherein at least one of the amino acids of the fragment is a D-amino acids. In a more particular embodiment, peptide fragment F2 has a sequence selected from the group consisting of SEQ ID NO. 91, 13, 92-106, wherein all the amino acids of the fragment are D-amino acids. In another particular embodiment of the peptide of the invention, peptide fragment F1 has a sequence selected from the group consisting of SEQ ID NO. 107-124, 179 wherein at least one of the amino acids of the fragment is a D-amino acids. In a more particular embodiment, peptide fragment F1 has a sequence selected from the group consisting of SEQ ID NO. 107-124, 179, wherein all the amino acids of the fragment are D-amino acids.

In a particular embodiment, the peptide of the invention comprises peptide fragments F1, F2 and F3. In another embodiment, the peptide of the invention comprises peptide fragments F1 and F2. In another embodiment, the peptide of the invention comprises peptide fragments F1 and F3.

In another embodiment, the peptide of the invention does not comprise fragment F2. In another embodiment, the peptide of the invention does not comprise peptide fragment F3.

In another embodiment, the peptide of the invention consists of peptide fragments F1, F2 and F3. In another embodiment, the peptide of the invention consists of peptide fragments F1 and F2. In another embodiment, the peptide of the invention consists of peptide fragments F1 and F3.

In another embodiment, each one of the amino acids of the peptide of the invention is an L- or a D-amino acid. In another embodiment, the peptide of the invention comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, preferably at least 1 D-amino acid and the rest of amino acids are L-amino acids. In another embodiment, the peptide of the invention comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, particularly 1 D-amino acid and the rest of amino acids are L-amino acids. In another embodiment, all the amino acids of the peptide of the invention are D-amino acids. In another embodiment, all the amino acids of the peptide of the invention are L-amino acids.

In another embodiment, each one of the amino acids of peptide fragment F1 as defined herein is an L- or a D-amino acid. In another embodiment peptide fragment F1 as defined herein comprises at least one, 2, 3, 4, 5, 6, 7, 8, 9, particularly at least 1 D-amino acid, and the rest of amino acids of the peptide fragment are L-amino acids. In another embodiment, peptide fragment F1 as defined herein comprises 1, 2, 3, 4, 5, 6, 7, 8, 9, particularly 1 D-amino acid, and the rest of amino acids of the peptide fragment are L-amino acids. In a particular embodiment, all the amino acids of the peptide fragment F1 as defined herein are D-amino acids. In another embodiment, all the amnio acids of peptide fragment F1 are L-amino acids.

In another embodiment, each one of the amino acids of peptide fragment F2 as defined herein is an L- or a D-amino acid. In another embodiment peptide fragment F2 as defined herein comprises at least one, 2, 3, 4, 5, 6, 7, 8, 9, 10, 11, 12, 13, 14, 15, 16, 17 or 18, particularly at least 1 D-amino acid, and the rest of amino acids of the peptide fragment are L-amino acids. In another embodiment, peptide fragment F2 as defined herein comprises 1, 2, 3, 4, 5, 6, 7, 8, 9, 10, 11, 12, 13, 14, 15, 16, 17, 18, particularly 1 D-amino acid, and the rest of amino acids of the peptide fragment are L-amino acids. In a particular embodiment, all the amino acids of the peptide fragment F2 as defined herein are D-amino acids. In another embodiment, all the amino acids of peptide fragment F2 are L-amino acids.

In another embodiment, each one of the amino acids of peptide fragment F3 as defined herein is an L- or a D-amino acid. In another embodiment peptide fragment F3 as defined herein comprises at least one, 2, 3, 4, 5, 6, 7, 8, 9, 10, 11, 12, 13, 14, 15, 16, 17, 18 or 19, particularly at least 1 D-amino acid, and the rest of amino acids of the peptide fragment are L-amino acids. In another embodiment, peptide fragment F2 as defined herein comprises 1, 2, 3, 4, 5, 6, 7, 8, 9, 10, 11, 12, 13, 14, 15, 16, 17, 18 or 19, particularly 1 D-amino acid, and the rest of amino acids of the peptide fragment are L-amino acids. In a particular embodiment, all the amino acids of the peptide fragment F3 as defined herein are D-amino acids. In another particular embodiment, all the amino acids of peptide fragment F3 are L-amino acids.

In another particular embodiment, the peptide comprises F2, F1 and F3, and each one of the amino acids of peptide fragments F2 and F3 is an L- or a D-amino acid and all amino acids in fragment Flare L-amino acids, preferably wherein fragment F1 has sequence SEQ ID NO.9 or SEQ ID NO.124. In another embodiment the peptide comprises F2, F1 and F3, and at least one amino acid is a D-amino acid in fragments F2 and F3, and all amino acids are L-amino acids in fragment F1, preferably wherein fragment F1 has sequence SEQ ID NO.9 or SEQ ID NO.124. In another particular embodiment, the peptide comprises F2 and F3, and all amino acid are a D-amino acid in fragments F2 and F3, and all amino acids are L-amino acids in fragment F1, preferably wherein fragment F1 has sequence SEQ ID NO.9 or SEQ ID NO.124.

In another particular embodiment, the peptide comprises fragments F2 and F1, and each one of the amino acids of peptide in fragments F2 is an L- or a D-amino acid, and all amino acids in fragment F1, preferably wherein fragment F1 are L-amino acids, preferably wherein fragment F1 has sequence SEQ ID NO.9 or SEQ ID NO.124. In another particular embodiment, the peptide comprises fragments F2 and F1 and at least one amino acid is a D-amino acid in fragment F2, and all amino acids are L-amino acids in fragment F1, preferably wherein fragment F1 has sequence SEQ ID NO.9 or SEQ ID NO.124. In another particular embodiment, the peptide comprises fragments F2 and F1, and all amino acid are a D-amino acid in fragment F2, and all amino acids are L-amino acids in fragment F1, preferably wherein fragment F1 has sequence SEQ ID NO 9 or SEQ ID NO.124.

In another particular embodiment, the peptide comprises fragments F3 and F1, and each one of the amino acids of peptide in fragments F3 is an L- or a D-amino acid, and all amino acids are L-amino acids in fragment F1, preferably wherein fragment F1 has sequence SEQ ID NO.9 or SEQ ID NO.124. In another particular embodiment, the peptide comprises fragments F3 and F1 and at least one amino acid is a D-amino acid in fragment F3, and all amino acids are L-amino acids in fragment F1, preferably wherein fragment F1 has sequence SEQ ID NO 9 or SEQ ID NO.124. In another particular embodiment, the peptide comprises fragments F3 and F1, and all amino acid are a D-amino acid in fragment F3, and all amino acids are L-amino acids in fragment F1, preferably wherein fragment F1 has sequence SEQ ID NO 9 or SEQ ID NO.124.

In a particular embodiment of the peptide of the invention, fragment F1 has SEQ ID NO. 1, SEQ ID NO. 5, SEQ ID NO. 6, SEQ ID NO. 7, SEQ ID NO. 8, SEQ ID NO. 9, SEQ ID NO. 10 or SEQ ID NO. 11, and at least one of the amino acids of the fragment is D-aa. In a particular embodiment of the peptide of the invention, fragment F2 has SEQ ID NO. 2, 12, 13 or 14, and and at least one of the amino acids of the fragment is D-aa. In another particular embodiment of the peptide of the invention, fragment F3 has SEQ ID NO. 4 or SEQ ID NO. 15 and at least one of the amino acids of the fragment is D-aa.

In a particular embodiment of the peptide of the invention, fragment F1 has SEQ ID NO. 1, SEQ ID NO. 5, SEQ ID NO. 6, SEQ ID NO. 7, SEQ ID NO. 8, SEQ ID NO. 9, SEQ ID NO. 10 or SEQ ID NO. 11, and all the amino acids of the fragment are D-aa. In a particular embodiment of the peptide of the invention, fragment F2 has SEQ ID NO. 2, 12, 13 or 14, and all the amino acids of the fragment are D-aa. In another particular embodiment of the peptide of the invention, fragment F3 has SEQ ID NO. 4 or SEQ ID NO. 15 and all the amino acids of the fragment are D-aa.

The term “D amino acid”, “D-aa” or “Daa” (wherein the amino acid (aa) can be any amino acid referred herein), refers to an amino acid having a D configuration, well known by an expert in the field.

The term “L amino acid”, “L-aa” or “Laa” (wherein the amino acid (aa) can be any amino acid referred herein), refers to an amino acid having an L configuration, well known by an expert in the field.

Unless otherwise stated, the one or more of the amino acids forming the peptide fragments or the peptide of the invention can have an L- or D-configuration.

In a particular embodiment, at least one amino acid of the peptide of the invention comprises a modification in its side chain. In a particular embodiment, said modification is the addition of a —C(O)R₇ group to the side chain of the peptide, being R₇ a (C₁-C₂₀)alkyl or a (C₁-C₂₀)alkenyl. In a more particular embodiment, R₇ is (C₁-C₂₀)alkyl. In an even more particularly embodiment, the modification is the addition of an acetyl group or a palmitoyl group. In another particular embodiment the modification is the addition of a —C(O)R₇ group to the side chain of the peptide, being R₇ a (C₁-C₂₀)alkenyl. In a more particular embodiment, the amino acid is a lysine or a tryptophane. In another particular embodiment, the modified amino acid is not at the N- nor in the C-terminal end position of the peptide. In a more particular embodiment, the modified amino acid is the lysine in fragment F2 with SEQ ID NO. 13 (TKTSSSFEVR). In an even more particular embodiment, the modified amino acid is the lysine in fragment F2 with SEQ ID NO. 13 (TKTSSSFEVR) and consists in the addition of an acetyl group. In another even more particular embodiment, the modified amino acid is lysine in fragment F2 with SEQ ID NO. 13 (TKTSSSFEVR) and consists in the addition of a palmitoyl group. In another embodiment, the modified amino acid is the tryptophane in fragment F1 with SEQ ID NO. 9: TWDPEGVIFY. In an even more particular embodiment, the modified amino acid is the tryptophane in fragment F1 with SEQ ID NO. 9: TWDPEGVIFY and consists in the addition of a palmitoyl group. In a yet more particular embodiment, at least one amino acid of the peptide fragment as defined in any of the embodiments in the present paragraph is a D-amino acid, even yet more particularly, all the amino acids of said fragment are a D-amino acid.

In an embodiment, the N-terminal end of the peptide of the invention corresponds to —NR₁R₂, being R₁ and R₂ independently selected from the group consisting of —H, —C(O)R₃, a triglyceride, (C₁-C₂₀)alkyl, particularly (C₁-C₁₅)alkyl, more particularly (C₁-C₁₀)alkyl and (C₁-C₂₀)alkenyl, particularly (C₁-C₁₅)alkenyl, more particularly (C₁-C₁₀)alkenyl. In a particular embodiment, the N-terminal end of the peptide of the invention corresponds to —NR₁R₂, being R₁ and R₂ independently selected from the group consisting of —H, —C(O)R₃, a triglyceride, (C₁-C₂₀)alkyl, (C₁-C₁₅)alkyl, (C₁-C₁₀)alkyl, (C₁-C₂₀)alkenyl, (C₁-C₁₅)alkenyl, and (C₁-C₁₀)alkenyl.

In one embodiment, the N terminal end of the peptide of the invention corresponds to NH₂ or —NHC(O)R₃, being R₃ a (C₁-C₂₀)alkyl, particularly being R₃ a (C₁-C₁₅)alkyl, more particularly being R₃ a (C₁-C₁₀)alkyl. In a particular embodiment, the N terminal end of the peptide of the invention corresponds to NH₂. In a particular embodiment, the N terminal end of the peptide of the invention corresponds to —NHC(O)R₃, being R₃ a (C₁-C₂₀)alkyl, preferably being R₃ a (C₁-C₁₅)alkyl, more preferably being R₃ a (C₁-C₁₀)alkyl.

In another embodiment, the N terminal end of the peptide of the invention corresponds to —NHC(O)R₃, being R₃ a (C₁-C₂₀)alkenyl, particularly being R₃ a (C₁-C₁₅)alkenyl, more particularly being R₃ a (C₁-C₁₀)alkenyl.

In another particular embodiment, the N terminal end of the peptide of the invention corresponds to —NHC(O)R₃ and R₃ is —CH₃. In another particular embodiment, the N terminal end of the peptide of the invention corresponds to —NHC(O)R₃ and R₃ is CH₃CH₂. In another particular embodiment, the N terminal end of the peptide of the invention corresponds to —NHC(O)R₃ and R₃ is CH₃(CH₂)₁₄.

In one embodiment of the first aspect of the invention, the C-terminal end of the peptide is —COOH, —C(O)NH₂ or —C(O)NHR₆, wherein R₆ is (C₁-C₂₀)alkyl or (C₁-C₂₀)alkenyl. In one embodiment of the first aspect of the invention, the C-terminal end of the peptide is —COOH or C(O)NH₂.

In one embodiment of the first aspect of the invention, the N terminal end of the peptide of the invention corresponds to NH₂ or —NHC(O)R₃, being R₃ a (C₁-C₁₀)alkyl; and the C-terminal end of the peptide is —COOH or —C(O)NH₂.

In another embodiment of the first aspect of the invention, the N terminal end of the peptide of the invention corresponds to —NHC(O)R₃, being R₃ a (C₁-C₁₀)alkenyl; and the C-terminal end of the peptide is —COOH or —C(O)NH₂.

In one embodiment of the first aspect of the invention, optionally in combination with any of the embodiments provided above or below, the N terminal end of the peptide of the invention corresponds to _{NH2}, and the C-terminal end of the peptide is —COOH, —C(O)NH₂ or —C(O)NHR₆, wherein R₆ is (C₁-C₂₀)alkyl, preferably wherein the C-terminal end of the peptide is —COOH or C(O)NH₂.

In one embodiment of the first aspect of the invention, the N terminal end of the peptide of the invention corresponds to NH₂; and the C-terminal end of the peptide is —C(O)NHR₆, wherein R₆ is (C₁-C₂₀)alkenyl.

In one embodiment of the first aspect of the invention the N terminal end of the peptide of the invention corresponds to —NHC(O)R₃, being R₃ a (C₁-C₂₀)alkyl, preferably being R₃ a (C₁-C₁₅)alkyl, more preferably being R₃ a (C₁-C₁₀)alkyl; and the C-terminal end of the peptide is —COOH, —C(O)NH₂ or —C(O)NHR₆, wherein R₆ is (C₁-C₂₀)alkyl, preferably wherein the C-terminal end of the peptide is —COOH or C(O)NH₂.

In another embodiment, the N terminal end of the peptide of the invention corresponds to —NHC(O)R₃, being R₃ a (C₁-C₂₀)alkenyl, preferably being R₃ a (C₁-C₁₅)alkenyl, more preferably being R₃ a (C₁-C₁₀)alkenyl; and the C-terminal end of the peptide is —COOH, —C(O)NH₂ or —C(O)NHR₆, wherein R₆ is (C₁-C₂₀)alkyl, preferably wherein the C-terminal end of the peptide is —COOH or C(O)NH₂.

In another embodiment, the N terminal end of the peptide of the invention corresponds to —NHC(O)R₃, being R₃ a (C₁-C₂₀)alkenyl, preferably being R₃ a (C₁-C₁₅)alkenyl, more preferably being R₃ a (C₁-C₁₀)alkenyl; and the C-terminal end of the peptide is —C(O)NHR₆, wherein R₆ is (C₁-C₂₀)alkenyl.

In one embodiment of the first aspect of the invention, the N terminal end of the peptide of the invention corresponds to —NHC(O)R₃ and R₃ is —CH₃; and the C-terminal end of the peptide is —COOH, —C(O)NH₂ or —C(O)NHR₆, wherein R₆ is (C₁-C₂₀)alkyl, preferably wherein the C-terminal end of the peptide is —COOH or C(O)NH₂.

In another embodiment, the N terminal end of the peptide of the invention corresponds to —NHC(O)R₃ and R₃ is —CH₃; and the C-terminal end of the peptide is —C(O)NHR₆, wherein R₆ is (C₁-C₂₀)alkenyl.

In one embodiment of the first aspect of the invention, the N terminal end of the peptide of the invention corresponds to —NHC(O)R₃ and R₃ is CH₃CH₂; and the C-terminal end of the peptide is —COOH, —C(O)NH₂ or —C(O)NHR₆, wherein R₆ is (C₁-C₂₀)alkyl, preferably wherein the C-terminal end of the peptide is —COOH or C(O)NH₂.

In one embodiment of the first aspect of the invention, the N terminal end of the peptide of the invention corresponds to —NHC(O)R₃ and R₃ is CH₃CH₂; and the C-terminal end of the peptide is —C(O)NHR₆, wherein R₆ is (C₁-C₂₀)alkenyl.

In one embodiment of the first aspect of the invention, optionally in combination with any of the embodiments provided above or below, the N terminal end of the peptide of the invention corresponds to —NHC(O)R₃, being R₃ CH₃(CH₂)₁₄; and the C-terminal end of the peptide is —COOH, —C(O)NH₂ or —C(O)NHR₆, wherein R₆ is (C₁-C₂₀)alkyl, preferably wherein the C-terminal end of the peptide is —COOH or C(O)NH₂.

In one embodiment of the first aspect of the invention, the N terminal end of the peptide of the invention corresponds to —NHC(O)R₃, being R₃ CH₃(CH₂)₁₄; and the C-terminal end of the peptide is —C(O)NHR₆, wherein R₆ is (C₁-C₂₀)alkenyl.

The term (C₁-C₂₀)alkyl refers to a saturated straight or branched alkyl chain having from 1 to 20 carbon atoms. Concomitantly, the terms (C₁-C₁₅)alkyl and (C₁-C₁₀)alkyl refer to saturated straight or branched alkyl chains having each from 1 to 15 carbon atoms, or 1 to 10 carbon atoms, respectively.

The term (C₁-C₂₀)alkenyl refers to mono- or poly-unsaturated straight or branched alkenyl chain having from 1 to 20 carbon atoms. Concomitantly, the terms (C₁-C₁₅)alkenyl and (C₁-C₁₀)alkenyl refer to mono- or poly-unsaturated straight or branched alkenyl chains having each from 1 to 15 carbon atoms, or 1 to 10 carbon atoms, respectively.

In a particular embodiment, the peptide of the invention has lipolytic activity.

The term “lipolytic activity” as used herein refers to the activity consisting in inducing lipolysis. Thus, a peptide with lipolytic activity, as used herein, refers to a peptide that induces lipolysis, in particular when administered to an animal body. As well known by an expert in the field, lipolysis is the enzymatic decomposition of triglycerides into glycerol and fatty acids. Methods to determine the lipolytic activity of a peptide as just defined are well-known by an expert in the field and include those disclosed in the examples of the present invention, such as in example 2-4 or 6. Non-limitative examples of such methods include adding the peptide of interest, optionally at different concentrations of for instance 1 μg/ml, 10 μg/ml, 25 μg/ml, 50 μg/ml, 75 μg/ml or 100 μg/ml, to a cell culture of differentiated adipocytes, such as mouse adipocytes, for instance differentiated 3T3-L1 cell line mouse adipocytes (Ref. JCRB9014-A) from Tebu-Bio (Tebu-bio, Barcelona, Spain) or human mature adipocytes. In parallel, a control compound is added to another cell culture of the same cell type at the same or comparable concentration as the peptide of interest has been added to the other cell culture. After a certain period of time, such as 12 or 16 hours, the supernatant of both cell cultures is collected (that with the peptide of interest and that with the control compound), and the glycerol content present in each supernatant is determined, with for instance Lipolysis Assay kit from Tebu-bio (LIP-1-NC). The amount of glycerol detected is considered to be the glycerol released by the cultured cells in the presence of the corresponding compound. If the amount of glycerol released by the cells cultured in the presence of the peptide of interest is equal or higher than that released by the cell cultured in the presence of the control compound, it is considered that the peptide of interest has lipolytic activity, in particular equal or higher than that of the control compound. In an embodiment, the control compound is isoproterenol or semaglutide.

The term “isoproterenol”, as used herein, refers to the compound with IUPAc name 4-[1-hydroxy-2-(propan-2-ylamino)ethyl]benzene-1,2-diol.

The term “semaglutide”, as used herein, refers to the compound with CAS number 910463-68-2.

In a particular embodiment, the peptide of the invention has lipolytic activity, wherein the lipolytic activity of the peptide is equal or higher than that of a control compound. In a particular embodiment, the control compound is is isoproterenol or semaglutide, particularly isoproterenol.

In a particular embodiment, the peptide of the invention has a lipolytic activity that is equal to that of a control compound if it is higher or lower than that of the control compound in less than 30%, 25%, 20%, 15%, 12%, 10%, 7%, 5%, 3%, 1% of that of a control compound.

In a particular embodiment, the peptide of the invention has a lipolytic activity that is equal to that of a control compound if the lipolytic activity of the peptide is at least 70%, 75%, 80%, 85%, 90%, 95%, 100%, particularly at least 80% of the lipolytic activity of the control compound.

In a particular embodiment, the peptide of the invention has a lipolytic activity higher than that of a control compound if it is at least 5%, 10%, 15%, 20%, 25%, 30%, 35%, 40%, 45%, 60%, 70%, 80%, 90%, 100%, 120%, 150%, 200%, 250%, 300%, 350%, 400%, 450%, 500%, 550%, 600%, particularly at least 30%, higher than that of said control compound.

Methods to determine if a peptide has lipolytic activity have been provided above in the definition of lipolytic activity.

In a particular embodiment, the lipolytic activity as referred herein for the peptide of the invention is measured by a method that determines the amount of glycerol released by differentiated adipocytes in culture with a certain concentration of the peptide of the invention, such as 100 μg/ml, for a period of time of 2 h-16 h, preferably 16 h, and compares said amount of glycerol released with that released by a cell culture of the same cell type in culture with the same amount of a control compound for the same amount of time, wherein if the amount of glycerol released by the cells in culture with the peptide of the invention is equal or higher than that released by the cells in culture with the control compound, the peptide is considered to have lipolytic activity. In a particular embodiment, the control compound is isoproterenol or semaglutide, particularly isoproterenol. In a particular embodiment, the amount of glycerol released by the cells in culture with the peptide of the invention equal or higher than that released by the cells in culture with the control compound is any of the percentages indicated above for an equal or higher lipolytic activity of the peptide as compared to a control peptide, wherein the lipolytic activity is substituted by the amount of glycerol released by the corresponding cells.

In a particular embodiment, the peptide of the invention has anti-lipogenic activity.

The term “anti-lipogenic activity”, or “inhibition of the lipogenesis” as used herein, refers to the activity that consist in avoiding the formation of triglycerides from the enzymatic condensation of glycerol and fatty acids, in particular in an animal body. Thus, a peptide with anti-lipogenic activity, as used herein, refers to a peptide that avoids, or inhibits, the formation of triglycerides from the enzymatic condensation of glycerol and fatty acids, in particular in an animal body. Methods to determine the anti-lipogenic activity of a peptide as just defined are well known by an expert in the field and include those disclosed in the examples of the present invention, such as in examples 2, 3, 4 or 6 of the present invention. A non-limitative example of a method to determine if a peptide has anti-lipogenic activity includes adding the peptide of interest, optionally at different concentrations of for instance 1 μg/ml, 10 μg/ml, 25 μg/ml, 50 μg/ml, 75 μg/ml or 100 μg/ml, to a cell culture of HepG2 cells treated with fructose to induce lipogenesis (such as 30 mM of fructose). In parallel, a first control cell culture of HepG2 cells treated with fructose is also prepared, and a second control cell culture of HepG2 cells treated with fructose is also prepared in which a control compound is added at the same or comparable concentration as the peptide of interest has been added to the other cell culture. The control compound can be for instance telmisartan or semaglutide. After a certain period of time, such as 5 days, the expression level, particularly in serum, of key proteins involved in lipogenesis, such as PPARγ transcription factor, Acetyl-CoA carboxylase (ACC) enzymes is determined in the cells of each of the cell cultures. If the expression level of said enzyme/s is/are reduced in the cell culture in the presence of the peptide of the invention at an equal or higher level than in the cell culture in the presence of the control compound, as compared to in the first control cell culture, it is considered that the peptide has anti-lipogenic activity. Methods to determine the expression level of specific proteins in the cells are well-known by an expert in the field and include RT-PCR, or RNA sequencing. Other methods include detection of the level of protein in the cells using specific antibodies against said protein, for instance in a western blot, ELISA or by mass spectrometry.

The term “telmisartan” as used herein, refers to the compound with CAS number 144701-48-4.

In a particular embodiment, the peptide of the invention has anti-lipogenic activity, wherein the anti-lipogenic activity of the peptide is equal or higher than that of a control compound. In a particular embodiment, the control compound is telmisartan or semaglutide, particularly selmaglutide. In another particular embodiment, the control compound is telmisartan.

In a particular embodiment, the peptide of the invention has an anti-lipogenic activity that is equal to that of a control compound if it is higher or lower than that of said control compound in less than 30%, 25%, 20%, 15%, 12%, 10%, 7%, 5%, 3%, 1% of that of a control compound.

In a particular embodiment, the peptide of the invention has an anti-lipogenic activity that is equal to that of a control compound if the anti-lipogenic activity of the peptide is at least 70%, 75%, 80%, 85%, 90%, 95%, 100%, particularly at least 80% of the anti-lipogenic activity of the control compound.

In a particular embodiment, the peptide of the invention has an anti-lipogenic activity higher than that of a control compound if it is at least 5%, 10%, 15%, 20%, 25%, 30%, 35%, 40%, 45%, 60%, 70%, 80%, 90%, 100%, 120%, 150%, 200%, 250%, 300%, 350%, 400%, 450%, 500%, 550%, 600%, 700%, 1000% preferably at least 50%, higher than that of said control compound. Methods to determine the anti-lipogenic activity of a peptide or compound have been provided above in the definition of “anti-lipogenic activity”.

In a particular embodiment, the anti-lipogenic activity as referred herein for the peptide of the invention is measured by a method that compares the expression level of anti-lipogenic proteins, such as PPARγ, ACC and/or FAS in cells, particularly in HEPG2 cells in culture for 2-7 days, particularly 5 days, with a certain concentration of the peptide of the invention, such as 100 μg/ml, and with induced lipogenesis, particularly by adding fructose, more particularly 30 mM of fructose, into the cell culture, with the expression level of said protein/s in cells in culture with a control compound for the same amount of time with induced lipogenesis as well, wherein if the expression level and if the expression level of said proteins is lower in the cells in culture with the peptide of the invention than in in the cells in culture with the control compound, it is considered that the peptide of the invention has anti-lipogenic activity. In a particular embodiment, the term “expression level of said proteins lower than” or “expression level of any of said proteins is reduced as compared to”, as used herein, refers to an expression level that is at least 5%, 10%, 15%, 20%, 25%, 30%, 35%, 40%, 45%, 60%, 70%, 80%, 90%, 100%, 120%, 150%, 200%, 250%, 300%, 350%, 400%, 450%, 500%, 550%, 600%, 700%, 1000% preferably at least 50%, lower than the corresponding reference level.

Methods to determine the expression level of a protein in cells have been provided above in the definition of “anti-lipogenic activity”.

In an embodiment, the peptide of the invention has antifibrotic activity.

The term “antifibrotic”, as used herein, refers to the activity consisting in reducing or inhibiting fibrosis.

Fibrosis is defined by the overgrowth, hardening, and/or scarring of various tissues and is attributed to excess deposition of extracellular matrix components including collagen. Repeated injuries, chronic inflammation and repair are susceptible to fibrosis, where an accidental excessive accumulation of extracellular matrix components, such as collagen, is produced by fibroblasts, leading to the formation of a permanent fibrotic scar. Indeed, fibrosis is a pathological feature of most chronic inflammatory diseases.

Thus, antifibrotic activity, in the context of a compound or the peptide of the invention, refers to the capacity of said compound or peptide to reduce the formation of fibrous tissue, or the excess deposition of extracellular matrix components including collagen in a tissue. In a particular embodiment, the antifibrotic activity refers to the inhibition of the formation of the fibrotic tissue, or of the excess deposition of extracellular components, such as collagen. In another embodiment, the antifibrotic activity refers to the reduction in the amount of fibrotic tissue formed, or of the excessive extracellular components deposited, such as of collagen.

Methods to determine if a peptide has antifibrotic activity are well known by an expert in the filed and include those disclosed in the examples of the present invention, such as in examples 2-6 of the present invention. Non-limitative examples of said methods include determining, upon subcutaneous administration of the peptide of interest for a certain time (for instance 20 mg/kg/day of the peptide of the invention during 10 weeks) to a mouse model of fibrosis (for instance a mouse model of fibrosis wherein fibrosis is induced by twice a week intraperitoneal injection of CCl4 (1 ml/Kg diluted in olive oil 1:5) during a 4 weeks period), the expression level in serum of collagen. In case the expression level of collagen is reduced upon administration of the peptide as compared to the lack of administration of the peptide, or to the administration of a negative control (such as the vehicle used for the administration of the peptide), it is considered that the peptide has antifibrotic activity. Methods to determine the expression level of specific proteins in the serum of mouse are well-known by an expert in the field and include determining the mRNA expression level in serum samples isolated from the treated and control mice by RT-PCR or RNA sequencing. Other methods include detection of the level of protein in serum using specific antibodies against said protein, for instance in a western blot, ELISA or by mass spectrometry.

Alternative or additional methods include comparing the collagen deposition in the fibrotic tissue formed in the fibrosis mouse model treated with the negative control (or not treated with the peptide of the invention), and the collagen deposited in the same tissue in the mouse treated with the peptide of interest. Histological studies using picrosirius staining (collagen staining) can be used for that purpose. If there is a reduction in the picrosirius staining intensity or in the picrosirius stained surface, it is considered that the peptide of the invention has antifibrotic activity.

In a particular embodiment, the antifibrotic activity as referred herein for the peptide of the invention is measured by a method that compares the expression level of collagen in an isolated serum sample from a mouse model of fibrosis, particularly in a mouse with fibrosis induced fibrosis induced by injection of CCl4, particularly a twice a week intraperitoneal injection of CCl4 (1 ml/Kg diluted in olive oil 1:5) during a 4 weeks period, and that has received subcutaneous administration of 20 mg/kg/day of the peptide of the invention during 8 weeks to that in an isolated serum sample from a corresponding control mouse that has not received the peptide of the invention, or that has received a negative control (such as the vehicle used in the administration of the peptide of the invention), and if the expression level of collagen is lower in the serum sample of the mouse treated with the peptide of the invention than in the serum sample of the mouse not treated with the peptide of the invention (or treated with the negative control), it is considered that the peptide of the invention has antifibrotic activity. In a particular embodiment, the expression “expression level of collagen lower than”, as used herein, refers to an expression level that is at least 5%, 10%, 15%, 20%, 25%, 30%, 35%, 40%, 45%, 60%, 70%, 80%, 90%, 100%, 120%, 150%, 200%, 250%, 300%, 350%, 400%, 450%, 500%, 550%, 600%, preferably at least 20%, lower than the corresponding reference level. Methods to determine the expression level of a protein in a serum sample have been provided above in the definition of “anti-lipogenic activity”.

In an embodiment, the peptide of the invention has anti-inflammatory activity.

The term “anti-inflammatory activity” as used herein, refers to the activity consisting in reducing inflammation or swelling in an animal body. Inflammation is part of the complex biological response of body tissues to harmful stimuli, such as damaged cells, irritants, or pathogens, and is a protective response involving immune cells, blood vessels, and molecular mediators. The five cardinal signs are heat, pain, redness, swelling, and loss of function. The function of inflammation is to eliminate the initial cause of cell injury, clear out necrotic cells and tissues damaged from the original insult and the inflammatory process, and initiate tissue repair. Too little inflammation could lead to progressive tissue destruction by the harmful stimulus (e.g. bacteria) and compromise the survival of the organism. In contrast, too much inflammation, in the form of chronic inflammation, is associated with various diseases, such as hay fever, periodontal disease, atherosclerosis, and osteoarthritis.

Thus, a peptide with anti-inflammatory activity as used herein, refers to a peptide that reduces an inflammatory response of the body, in particular, the inflammation signs in a part of the body, such as heat, pain, redness, swelling, and loss of function. Methods to determine if a peptide has anti-inflammatory activity are well-known by an expert in the field and include those disclosed in the examples of the invention, such as in examples 4 or 6 of the present invention. Non-limitative examples of such methods include determining, upon subcutaneous administration of the peptide of interest for a certain time (for instance 20 mg/kg/day of the peptide of the invention during 10 weeks) to a mouse model of a disease that is associated to inflammation (such as of obesity or fatty liver disease, induced for instance by high fat diet feeding), the expression level in serum of protein markers of inflammation, such as F480. In case hepatic inflammation is to be determined, the hepatic expression of proinflammatory cytokines such as TNFα, IL1β and/or IL6 is determined. In case adipose tissue inflammation is to be determined, the expression level of IL1β, leptin and/or adiponectin are determined.

In case the expression level of said proteins is reduced, or the expression level of adiponectin is increased, upon administration of the peptide as compared to the lack of administration of the peptide, or to the administration of a negative control (such as the vehicle used for the administration of the peptide), it is considered that the peptide has anti-inflammatory activity. Methods to determine the expression level of a protein in a serum sample have been provided above in the definition of anti-lipogenic activity.

In a particular embodiment, the anti-inflammatory activity as referred herein for the peptide of the invention is measured by a method that compares the expression level of protein markers of inflammation, such as F480, TNFα, IL1β, IL6, leptin and/or adiponectin, in an isolated serum sample from a mouse model of a diseases associated to inflammation, particularly obesity or fatty liver, more particularly induced by high fat diet feeding, that has received subcutaneous administration of 20 mg/kg/day of the peptide of the invention during 8 weeks to that in an isolated serum sample from a corresponding control mouse that has not received the peptide of the invention, or that has received a negative control (such as the vehicle used in the administration of the peptide of the invention), and if the expression level of said protein F480, TNFα, IL1β, IL6, and/or leptin is lower, and/or the expression level of adiponectin is higher, in the serum sample of the mouse treated with the peptide of the invention than in the serum sample of the mouse not treated with the peptide of the invention (or treated with the negative control), it is considered that the peptide of the invention has anti-inflammatory activity. In a particular embodiment, the expression “expression level of said proteins lower than”, as used herein, refers to an expression level that is at least 5%, 10%, 15%, 20%, 25%, 30%, 35%, 40%, 45%, 60%, 70%, 80%, 90%, 100%, 120%, 150%, 200%, 250%, 300%, 350%, 400%, 450%, 500%, 550%, 600%, preferably at least 20%, lower than the corresponding reference level. In another embodiment, the expression “expression level of adiponectin is higher than”, as used herein, refers to an expression level that is at least 5%, 10%, 15%, 20%, 25%, 30%, 35%, 40%, 45%, 60%, 70%, 80%, 90%, 100%, 120%, 150%, 200%, 250%, 300%, 350%, 400%, 450%, 500%, 550%, 600%, preferably at least 20%, higher than the corresponding reference level. Methods to determine the expression level of a protein in a serum sample have been provided above in the definition of “anti-lipogenic activity”.

In a particular embodiment, the peptide of the invention has lipolytic activity, anti-lipogenic activity, anti-fibrotic and/or anti-inflammatory activity, as just defined. In a more particular embodiment, the peptide of the invention has lipolytic activity, anti-lipogenic activity, anti-fibrotic and/or anti-inflammatory activity. In another particular embodiment, the peptide of the invention has anti-lipogenic, anti-fibrotic and anti-inflammatory activity.

In a particular embodiment, the peptide of the invention has a lipolytic activity equal or higher than that of a control compound, and anti-lipogenic activity equal or higher than a control compound. In another particular embodiment, the peptide of the invention has a lipolytic activity equal or higher than that of a control compound, wherein said control compound is isoproterenol or semaglutide, more particularly isoproterenol, and anti-lipogenic activity equal or higher than a control compound, wherein said control compound is telmisartan or semaglutide, more particularly semaglutide.

In a particular embodiment, all the definitions and embodiments addressed to the peptide of the invention also apply to the pharmaceutically acceptable salt thereof as defined above, or the pharmaceutically acceptable salt of the peptide of the invention, or the pharmaceutically acceptable salt of the invention.

### II—Pharmaceutical Composition of the Invention

In a second aspect, the invention relates to a pharmaceutical composition comprising a therapeutical effective amount of the peptide, or pharmaceutically acceptable salt thereof, of the first aspect of the invention and one or more therapeutically acceptable carriers.

The term pharmaceutically acceptable excipient”, or “pharmaceutically acceptable carrier”, “pharmaceutically acceptable diluent”, or “pharmaceutically acceptable vehicle”, as used interchangeably herein, refers to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc., must also be “acceptable” in the sense of being compatible with the other ingredients of the formulation. Suitable carriers, excipients, etc. can be found in standard pharmaceutical texts, and include, as a way of example preservatives, agglutinants, humectants, emollients, and antioxidants.

The election of the pharmaceutical formulation will depend upon the nature of the active compound and its route of administration. Any route of administration may be used. In some embodiments, the route of administration is parenteral, and the composition is then appropriate for parenteral administration. In a particular embodiment, the route of administration is by injection. In a more particular embodiment, the route of administration is systemic, for example, by intramuscular, intravenous, intraarterial, intraperitoneal, subcutaneous, or transdermal injection. Topical administration is also contemplated, such that the pharmaceutical composition may be a topical composition.

The pharmaceutical compositions may be in any form, including, among others, tablets, pellets, capsules, aqueous or oily solutions, suspensions, emulsions, aerosols, or dry powdered forms suitable for reconstitution with water or other suitable liquid medium before use, for immediate or retarded release.

The term “therapeutically effective amount” as used herein, means an amount of an active agent high enough to deliver the desired benefit (either the treatment or prevention of the illness), but low enough to avoid serious side effects within the scope of medical judgment.

In a certain embodiment, the definitions and embodiments described in the first aspect of the invention are equally applicable to the second aspect of the invention.

### III—Medical Use of the Invention

In a third aspect, the invention relates to the peptide, or a pharmaceutically acceptable salt thereof, according to the first aspect of the invention, or the pharmaceutical composition according to the second aspect of the invention, for use in therapy.

This aspect of the invention can also be formulated as the use of the peptide, or a pharmaceutically acceptable salt thereof, of the first aspect of the invention, or the pharmaceutical composition according to the second aspect of the invention, for the preparation of a medicament. The present invention also relates to a method of treatment and/or prevention of a condition or disease comprising administering a therapeutically effective amount of the peptide of the invention, particularly together with pharmaceutically acceptable excipients and/or carriers, or the pharmaceutical composition of the invention to a subject in need thereof. In a particular embodiment, the subject is a mammal, more particularly a human.

In a fourth aspect, the invention relates to the peptide, or a pharmaceutically acceptable salt thereof, of the first aspect of the invention, or the pharmaceutical composition according to the second aspect of the invention, for use in the treatment and/or prevention of a condition or disease caused or associated to the accumulation of fat in an organ or tissue, particularly in an organ, inflammation and/or fibrosis, in particular in a mammal, more particularly in a human.

This aspect of the invention can also be formulated as the use of the peptide, or a pharmaceutically acceptable salt thereof, of the first aspect of the invention, or the pharmaceutical composition according to the second aspect of the invention, for the preparation of a medicament for the treatment and/or prevention of a condition or disease caused or associated to the accumulation of fat in an organ or tissue, inflammation and/or fibrosis. The present invention also relates to a method of treatment and/or prevention of a condition or disease caused or associated to the accumulation of fat in an organ or tissue, inflammation and/or fibrosis comprising administering a therapeutically effective amount of the peptide of the invention, particularly together with pharmaceutically acceptable excipients and/or carriers, or the pharmaceutical composition of the invention to a subject in need thereof. In a particular embodiment, the subject is a mammal, more particularly a human,

The term “disease or condition caused or associated to the accumulation of fat in an organ”, as used herein, refers to any disease characterized in that lipids are accumulated in an organ, in particular, associated to the accumulation of visceral fat. The term “disease or condition caused or associated to the accumulation of fat in a tissue”, refers to any disease characterized in that lipids are accumulated in a tissue, particularly selected from the list consisting of connective tissue, epithelial tissue, muscle tissue. In a particular embodiment, the disease or condition caused or associated to the accumulation of fat in an organ is characterized in an increased in the amount of visceral adipose tissue (VAT) in a subject.

In a particular embodiment, the disease or condition caused or associated to the accumulation of fat in an organ is selected from the list consisting of NAFLD, obesity, overweight, diabetes, lipohyperdystrophyand cardiovascular disease.

The term “NALFD” or “non-alcoholic fatty liver disease” as used herein, refers to a range of conditions caused by too much fat stored in the liver in people who drink little or no alcohol. Healthy liver cells should contain little or no fat. An essential criterion for the histological definition of NAFLD is the presence of lipid droplets in >5% of hepatocytes. NAFLD is also defined when more than 5% of liver weight or volume corresponds to intrahepatic fat.

The main stages of NAFLD are (i) fatty liver (steatosis), (ii) non-alcoholic steatohepatitis (NASH), (iii) hepatic fibrosis, (iv) cirrhosis.

The term “steatosis” or “fatty liver”, as used herein is a condition characterized in that the liver cells start to build-up fat, although there is no inflammation or scarring at this stage. There are often no symptoms in this early stage. Approximately 20% of subjects with fatty liver, will develop non-alcoholic steatohepatitis or NASH.

The term “non-alcoholic steatohepatitis” or “NASH”, as used herein, is a condition corresponding to the stage of NAFLD in which the build-up of fat in the liver cells is accompanied with inflammation. Inflammation occurs when the liver is repairing damaged tissue. If the amount of damaged tissue increases, the liver may eventually not be able to repair it efficiently and the inflamed tissue will be modified into a scar, giving rise to hepatic fibrosis.

The term “hepatic fibrosis”, as used herein, is a condition characterized persistent inflammation that leads to scar tissue formation around the liver and nearby blood vessels. In this stage the liver is still able to function appropriately. Removing or treating the cause of the inflammation may prevent further progression or even reverse some of the damage. However, if over time, the scar tissue starts to replace a significant part of the normal liver tissue, the liver function ends up being affected, which ultimately leads to cirrhosis.

The term “cirrhosis”, as used herein is the most severe stage of NAFLD, occurring after years of inflammation, where the liver becomes scarred and and permanently damaged; this permanent damage and can lead to liver failure (where the liver stops working properly) and liver cancer.

Thus, in a particular embodiment of the fourth aspect, the disease or condition caused or associated to the accumulation of fat in an organ is NAFLD, in particular steatosis, NASH, hepatic fibrosis or cirrhosis, more particularly NASH. In a particular embodiment, the disease or condition caused or associated to the accumulation of fat in an organ is NASH.

The term “obesity”, as used herein refers to an abnormal or excessive fat accumulation in a subject, in particular a mammal, more in particular a human, that presents a risk to health. The BMI associated to obesity in humans is of 30 or more. Obesity is frequently subdivided into 3 categories:
Class 1 obesity: BMI of 30 to <35
Class 2 obesity: BMI of 35 to <40
Class 3 obesity: BMI of 40 or higher. Class 3 obesity is sometimes categorized as “severe” obesity.

The term “body Mass Index” or “BMI”, as used herein refers to a subject's weight in kilograms (or pounds) divided by the square of height in meters (or feet).

In a particular embodiment of the fourth aspect, the disease or condition caused or associated to the accumulation of fat in an organ is obesity, in particular class 1 obesity, class 2 obesity or class 3 obesity. In a particular embodiment, the disease is class 1 obesity. In another embodiment, the disease is class 2 obesity. In another embodiment, the disease is class 3 obesity. In another embodiment, the disease is class 1 or class 2 obesity. In another embodiment, the disease is class 2 or class 3 obesity.

The term “overweight”, as used herein, refers to an abnormal or excessive fat accumulation in a subject, in particular a mammal, more in particular a human, that presents a risk to health, that is associated in humans to a BMI as defined above of between 25 and 30.

In a particular embodiment of the fourth aspect, the disease or condition caused or associated to the accumulation of fat in an organ is overweight.

In a particular embodiment of the fourth aspect, the disease or condition caused or associated to the accumulation of fat in an organ is NAFLD, obesity or overweight, preferably NASH, cirrhosis, obesity or overweight. In another particular embodiment of the medical use of the invention, the disease or condition caused or associated to the accumulation of fat in an organ is NAFLD or obesity, particularly NASH, cirrhosis, or obesity.

The term “diabetes”, or “diabetes mellitus”, as used herein, refers to is a chronic disease that occurs either when the pancreas does not produce enough insulin or when the body cannot effectively use the insulin it produces. Insulin is a hormone that regulates blood glucose. Hyperglycaemia, also called raised blood glucose or raised blood sugar, is a common effect of uncontrolled diabetes and over time leads to serious damage to many of the body's systems, especially the nerves and blood vessels. The most common form of diabetes is diabetes type 2. The term “type 2 diabetes”, as used herein, refers to a diabetes type characterized by insulin resistance, which may be combined with relatively reduced insulin secretion by the pancreas. In a particular embodiment of the fourth aspect, the disease or condition caused or associated to the accumulation of fat in an organ is diabetes. In a more particular embodiment, it is diabetes type 2.

The term “cardiovascular disease”, as used herein, refers to conditions affecting the heart or blood vessels. It's usually associated with a build-up of fatty deposits inside the arteries (atherosclerosis) and an increased risk of blood clots.

The term “lipohyperdistrophy”, as used herein, refers to localized abnormal fat accumulation, most commonly of the intra-abdominal compartment (visceral adipose tissue [VAT]), breasts, dorso-cervical area (buffalo hump), and/or as discrete accumulations under the skin (lipomas).

The term “disease caused or associated to inflammation”, as used herein, refers to a disease characterized in that inflammation occurs in a part of the body, wherein inflammation is as defined above in the first aspect of the invention, and does not disappear, or the body does not return to a healthy non-inflamed state. Non-limitative examples of inflammatory diseases include type 2 diabetes and rheumatoid arthritis.

The term “Rheumatoid arthritis” or “RA”, as used herein, refers to a long-term autoimmune disorder that primarily affects joints that typically results in warm, swollen, and painful joints. Pain and stiffness often worsen following rest. Most commonly, the wrist and hands are involved, with the same joints typically involved on both sides of the body. The disease may also affect other parts of the body, including skin, eyes, lungs, heart, nerves and blood. This may result in a low red blood cell count, inflammation around the lungs, and inflammation around the heart.[1] Fever and low energy may also be present. Often, symptoms come on gradually over weeks to months.

The term “a disease associated or caused by fibrosis”, or “fibrosis”, as used herein, refers to a disease characterized in the appearance of fibrotic tissue, as indicated in the definition of “fibrosis” in the first aspect of the invention. Non-limitative examples of fibrotic diseases include hepatic fibrosis, adipose tissue fibrosis, pulmonary fibrosis, liver cirrhosis, systemic sclerosis, kidney fibrosis, progressive kidney disease, and cardiovascular fibrosis. Thus, in a particular embodiment of the fourth aspect of the invention, the disease associated to or caused by fibrosis is hepatic fibrosis, adipose tissue fibrosis, pulmonary fibrosis, liver cirrhosis, systemic sclerosis, progressive kidney disease, kidney fibrosis, and cardiovascular fibrosis. In a more particular embodiment in a particular embodiment of the fourth aspect of the invention, the disease associated to or caused by fibrosis is hepatic fibrosis, adipose tissue fibrosis, pulmonary fibrosis kidney fibrosis, or cardiovascular fibrosis. In an even more particular embodiment, the disease associated to or caused by fibrosis is hepatic fibrosis or adipose tissue fibrosis, yet more particularly, hepatic fibrosis.

As well known by an expert in the field, fibrotic diseases can be associated with aging. In the young subjects, scars resolve over time and are replaced by new tissue. However, in older subjects, the scars do not always heal properly, and scar tissue can build up. Thus, in a particular embodiment of the fourth aspect of the invention, the disease associated to or caused by fibrosis is associated to aging. The term “is associated to aging”, as used herein, refers to the fact that it is the consequence of or results from the process of aging in a subject. Thus, in a particular embodiment, the disease associated to or caused by fibrosis, as defined in any of the embodiments above, results from the process of aging. In a more particular embodiment, the disease associated to or caused by fibrosis is hepatic fibrosis, adipose tissue fibrosis, pulmonary fibrosis, kidney fibrosis, or cardiovascular fibrosis, even more particularly hepatic fibrosis, and results from the process of aging. In a particular embodiment, the definitions and embodiments of the first and second aspects of the invention are equally applicable to the third and fourth aspects of the invention.

### IV— Cosmetic Composition of the Invention and Cosmetic Use of the Invention

In a fifth aspect, the invention relates to a cosmetic composition comprising the peptide, or pharmaceutically acceptable salt thereof, of the first aspect of the invention, and one or more cosmetically acceptable carriers.

The term “cosmetic composition” in accordance with the present invention relates to a formulation that can be used for cosmetic purposes. As well understood by a skilled person, cosmetic purposes or uses refer to any purpose or uses for improving the bodily appearance of an animal body, preferably of a mammal, more preferably a human. Said purposes and uses are thus non-therapeutic.

The term “cosmetically acceptable” refers to excipients or carriers that are appropriate for use in contact with human skin without inappropriate allergic response, instability, incompatibility, or toxicity among others.

In one embodiment, the cosmetic composition is a topical composition or an oral composition, particularly a topical composition.

In another particular embodiment, the form of the cosmetic composition is any of those indicated for the pharmaceutical composition in the second aspect of the invention.

In a sixth aspect, the invention relates to the use of the peptide, or a pharmaceutically acceptable salt thereof, of the first aspect of the invention, or of the cosmetic composition according to the fifth aspect of the invention, in cosmetics.

The cosmetic use, or the use in cosmetics, of the peptides and cosmetic composition according to the invention is to be applied to mammals, in particular humans, which are healthy subjects that need not have any diseases. That is, they can be subjects without a disease or condition associated with the accumulation of fat as defined above. Thus, the cosmetic use is only for esthetical purposes aiming to improve the bodily appearance, and not to treat any disease. In general terms, the cosmetically effective amounts are different than therapeutically effective amounts, since in a cosmetic use the peptide has only to reach the fat accumulated or stored at subcutaneous level.

In an embodiment, the cosmetic use of the invention is non-therapeutic. In another embodiment, the cosmetic use of the invention is non-surgical. In another embodiment of the invention, the cosmetic use of the invention is non-therapeutic and non-surgical.

In an embodiment, the cosmetic effect of the peptides and cosmetic compositions of the invention, derive from the lipolytic and/or anti-lipogenic effect provided by them. Therefore, the topical or dermal or oral, particularly dermal, administration of the peptides of the invention leads to the reduction of the subcutaneous fat herniated or accumulated within the fibrous connective tissue, such as cellulite. Subcutaneous fat is found just beneath the skin, as opposed to visceral fat, which is found in the peritoneal cavity. Subcutaneous fat can be measured using body fat calipers giving a rough estimate of total body adiposity. This fat aids in the process of homeostasis, by forming a layer of insulation to slow heat loss.

In particular embodiment, the cosmetic use of the invention is for reducing the subcutaneous fat in a mammal, preferably accumulated within the fibrous connective tissue under the skin of the mammal. In a particular embodiment, the cosmetic use of the invention is for reducing at least 5%, 10%, 20%, 30%, 40%, 50%, 60%, 70%, 80%, 90%, preferably at least 10% of the volume of the subcutaneous fat in a mammal, preferably accumulated within the fibrous connective tissue under the skin of the mammal. Methods to determine the volume of fat in a particular region of a body are well known by an expert in the field. Non-limitative examples of such methods include Magnetic resonance imaging (MRI), which allows to estimate the changes in adipose tissue volume before and after a treatment (such as the cosmetic non-therapeutic treatment of the invention). Subcutaneous fat can also be measured using body fat calipers.

The term “cellulite”, as used herein, refers to persistent subcutaneous fat causing dimpling of the skin or nodularity, often on the pelvic region (specifically the buttocks), lower limbs, and abdomen in females.

In another particular embodiment, the cosmetic use of the invention is for reducing cellulite in a mammal, preferably at least 5%, 10%, 20%, 30%, 40%, 50%, 60%, 70%, 80%, 90%, preferably at least 10% of the volume of the cellulite on a mammal. In a particular embodiment, the cellulite reduced is that located in any part of the body, such as the pelvic region (specifically the buttocks), lower limbs, or abdomen. Methods to determine the amount of cellulite in a region of the body are commonly known to an expert in the field and also include Magnetic resonance imaging (MRI) to determine the adipose tissue volume in a part of the body, allowing to estimate the changes in cellulite volume before and after a treatment (such as the cosmetic non-therapeutic treatment of the invention), or the use of body fat calipers.

In another particular aspect, the invention relates to a non-therapeutic method for reducing body weight in a subject with overweight, that comprises administering the peptide of the first aspect of the invention or the cosmetic composition of the fifth aspect of the invention, to said subject, wherein the subject is in particular a mammal, even more in particular a human. In a particular embodiment, the human is an adult human. In another particular embodiment, the human is a non-adult human. In another particular embodiment, the forms of the administered composition are as those disclosed in the second aspect of the invention for the pharmaceutical composition of the invention. In another particular embodiment, the route of administration is oral or dermal.

In a particular embodiment, reducing overweight in a subject with overweight involves reducing the BMI of the subject at least 5%, at least 10%, at least 15%, at least 20%, at least 25%, at least 30%, particularly at least 5%. In another particular embodiment, reducing overweight in a subject with overweight involves reducing the BMI of the subject at least 1, at least 2, at least 3, at least 4, at least 5, at least 6, at least 7 points, particularly at least 1 point.

In a particular embodiment, the definitions and embodiments of the first, second, third and fourth aspects of the invention are equally applicable to the fifth aspect of the invention.

### V— Kits and Devices of the Invention

In a seventh aspect, the invention relates to a kit of parts comprising:
a peptide, or pharmaceutically acceptable salt thereof, according to the first aspect of the invention, the pharmaceutical composition according to the second aspect of the invention, or the cosmetic composition according to the fifth aspect of the invention, and
a device selected from the group consisting of an injection device, an implant, or a biodegradable container.

The term “kit” as used herein, refers to a product comprising the products and/or compositions of the invention, packed so as to allow their transport and storage. In addition, the kits of the invention can contain instructions far the simultaneous, sequential or separate use of the different components which are in the kit. Said instructions can be in the form of printed material or in the form of an electronic support capable of storing instructions susceptible of being read or understood, such as, far example, electronic storage media (e.g. magnetic disks, tapes), or optical media (e.g. CD-ROM, DVD), or audio materials. Additionally or alternatively, the media can contain internet addresses that provide said instructions.

In an eighth aspect of the invention, the invention relates to a device, such as an injection device, a patch, or a biodegradable container, comprising the peptide or pharmaceutically acceptable salt thereof according to he first aspect, the pharmaceutical composition according to the second aspect of the invention, or the cosmetic composition according to the fifth aspect of the invention.

The term “injection device”, as used herein, refers to a medical device designed to deliver a dose of a particular drug. The injectors were initially designed to overcome the hesitation associated with self-administration of a needle-based drug delivery device. In a particular embodiment, the injection device is syringe, or one or more micro-needles.

The term “implant”, as used herein, refers to devices or tissues that are placed inside or on the surface of the body. As used herein, said devices allow delivering medication in the body region where they are placed.

In a particular embodiment, the implant is in the form of a patch.

The term “biodegradable container”, as used herein, refers to a device comprising degradable molecules in a more or less long time, often a few weeks to several months or even years after introduction into the skin.

In a particular embodiment, the definitions and embodiments of the first, second, third, fourth, fifth and sixth aspects of the invention are equally applicable to the seventh and eighth aspects of the invention.

Throughout the description and claims the word “comprise” and variations of the word, are not intended to exclude other technical features, additives, components, or steps. Furthermore, the word “comprise” encompasses the case of “consisting of”.

Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.
For completeness, the present description is also disclosed in the following numbered embodiments:
1—A peptide, or a pharmaceutically acceptable salt thereof, wherein the peptide has a length of 9 to 49 amino acids, each one of the amino acids being a L- or D-amino acid, and comprises:
a peptide fragment F1 with a length of 4-10 amino acids, each one of the amino acids being L- or D-amino acid, and with sequence

SEQ ID NO. 1:
(X¹)ₐ(X²)_{b}(X³)(X⁴)_{c}(X⁵)G(X⁶)_{d}(X⁷)ₑ(X⁸)_{f}(Y)_{g}

wherein:
X¹ represents T, or A,
X² represents W, N, Q, K or A,
X³ represents D or N,
X⁴ represents P, N, a basic amino acid or a hydrophobic amino acid, preferably P, N, L, V, or A,
X⁵ represents E or Q,
X⁶ and X⁷ are the same or different and represent A, V, L or I,
X⁸ represents F or P,
subindexes “a”, “b”, “c”, “d”, “e”, “f” and “g” are integer numbers independently selected from 0 and 1 and at least two of them represent 1,
wherein the peptide further comprises:
   i) a peptide fragment F2 at the N-terminal end of peptide fragment F1, with a length of 5 to 19 amino acids, each one of the amino acids being L- or D-amino acid, and that comprises sequence SEQ ID NO.2 (SF(X⁹)VR), wherein X⁹ represents E or Q,
   and/or
   ii) a peptide fragment F3 at the C-terminal end of peptide fragment F1, with a length of 10 to 20 amino acids, each one of the amino acids being L- or D-amino acid, and that comprises SEQ ID NO. 4 (GDTNPKDDWF).
2—The peptide or pharmaceutically acceptable salt thereof according to embodiment 1 wherein the peptide fragment F1 has a sequence SEQ ID NO. 5: (X¹)ₐ(X²)_{b}D (X⁴)E G(X⁶)_{d}(X⁷)ₑ(X⁸)_{f}(Y)_{g},
   wherein:
   X¹ represents T, or A,
   X² represents W, N, Q, K or A;
   X⁴ represents P, N or L,
   X⁶ and X⁷ are the same or different and represent A, V, L or I;
   X⁸ represents F or P,
   subindexes “a”, “b”, “d”, “e”, “f” and “g”” are integer numbers independently selected from 0 and 1.
3—The peptide or pharmaceutically acceptable salt thereof according to any one of embodiments 1-2, wherein fragment F1 has a sequence SEQ ID NO. 6: (X¹)ₐ(X²)_{b}D P E G(X⁶)_{d}(X⁷)ₑ(X⁸)_{f}(Y)_{g}
   wherein:
   X¹ represents T, or A,
   X² represents W, N, Q, K or A,
   X⁶ and X⁷ are the same or different and represent A, V, L or I,
   X⁸ represents F or P,
   Subindexes “a”, “b”, “d”, “e”, “f” and “g” are integer numbers independently selected from 0 and 1.
4—The peptide or pharmaceutically acceptable salt thereof according to any one of embodiments 1-2 wherein the peptide fragment F1 has a sequence SEQ ID NO. 7: (X¹)ₐ(X²)_{b}DLE G(X⁶)_{d}(X⁷)ₑ(X⁸)_{f}(Y)_{g},
   wherein:
   X¹ represents T, or A,
   X² represents W, N, Q, K or A;
   X⁶ and X⁷ are the same or different and represent A, V, L or I;
   X⁸ represents F or P,
   subindexes “a”, “b”, “d”, “e”, “f” and “g” are integer numbers independently selected from 0 and 1.
5—The peptide or pharmaceutically acceptable salt thereof according to any on of embodiments 1-2 wherein the peptide fragment F1 has a sequence SEQ ID NO. 8: (X¹)ₐ(X²)_{b}DNE G(X⁶)_{d}(X⁷)ₑ(X⁸)_{f}(Y)_{g},
   wherein:
   X¹ represents T, or A,
   X² represents W, N, Q K or A;
   X⁶ and X⁷ are the same or different and represent A, V, L or I;
   X⁸ represents F or P,
   subindexes “a”, “b”, “d”, “e”, “f” and “g” are integer numbers independently selected from 0 and 1.
6—The peptide, or pharmaceutically acceptable salt thereof according to any one of embodiments 1-3, wherein the peptide fragment F1 has sequence SEQ ID NO. 9: TWDPEGVIFY.
7—The peptide, or pharmaceutically acceptable salt thereof according to embodiment 1, wherein the peptide fragment F1 has a sequence selected from the group consisting of SEQ ID NO. 78-82.
8—The peptide, or pharmaceutically acceptable salt thereof according to embodiment 1, wherein the peptide fragment F1 has a length of 5 to 9 amino acids, each one of the amino acids being a L- or D-amino acid, and sequence SEQ ID NO.10 (T)ₐ(X²)_{b}D(X⁴)_{c}EG(X⁶)_{d}(X⁷)ₑ(X⁸)_{f}(Y)_{g}
   wherein:
   X² represents W, N, Q or K
   X⁴ represents P, N, a basic amino acid or a hydrophobic amino acid, preferably P, N, L, V, or A,
   X⁶ and X⁷ are the same or different and represent A, V, L or I,
   X⁸ represents F or P,
   subindexes “a”, “b”, “c”, “d” “e”, “f” and “g” are integer numbers independently selected from 0 and 1; provided that two, three, four, five or six of the subindexes “a”, “b”, “c” “d”, “e”, “f” and “g” represent 1; and the remaining subindexes represent zero.
9—The peptide, or pharmaceutically acceptable salt thereof, according to embodiment 8, wherein:
   X² represents N,
   X⁴ represents P, L, V, or A,
   X⁶ and X⁷ are the same or different and represent L, V, or A, and/or
   X⁸ represents F or P.
10—The peptide, or pharmaceutically acceptable salt thereof, according to any one of embodiments 1, 8-9, wherein the peptide fragment F1 has a sequence selected from the group consisting of SEQ ID NO. 67-76.
11—The peptide, or pharmaceutically acceptable salt thereof, according to any one of embodiments 1, or 8 wherein the peptide fragment F1 has a sequence from 5 to 9 amino acids, each one of the amino acids being L- or D-amino acid, and sequence SEQ ID NO.11

(T)ₐ(X²)_{b}D(X⁴)EG(X⁶)_{d}(X⁷)ₑ(F)_{f}(Y)_{g}

wherein:
X² represents W, N, Q or K,
X⁴ represents P, N or a basic amino acid,
X⁶ and X⁷ are the same or different and represent A, V, L or I,
subindexes “a”, “b”, “d” “e”, “f”, and “g” are integer numbers independently selected from 0 and 1; provided that one, two, three, four or five of the subindexes “a”, “b”, “d” “e”, “f”, and “g” represent 1; and the remaining subindexes represent zero.
12—The peptide, or pharmaceutically acceptable salt thereof according to embodiment 11, wherein:
   X² represents W or N,
   X⁴ represents P or N, and/or
   X⁶ or X⁷ represents V, L or I.
13—The peptide, or pharmaceutically acceptable salt thereof, according to any one of embodiments 1, 8, or 11-12 wherein the peptide fragment F1 has a sequence selected from the group consisting of: SEQ ID NO. 23-63.
14—The peptide, or pharmaceutically acceptable salt thereof, according to embodiment 1, wherein the peptide fragment F1 has SEQ ID NO. 108, particularly, has a sequence selected from the group consisting of SEQ ID NO. 107, 109-124, 179.
15—The peptide, or pharmaceutically acceptable salt thereof, according to any one of the previous embodiments, wherein the peptide fragment F2 comprises sequence SEQ ID NO. 12 (SFEVR).
16—The peptide, or pharmaceutically acceptable salt thereof, according to any one of the previous embodiments, wherein peptide fragment F2 comprises SEQ ID NO. 13 (TKTSSSFEVR).
17—The peptide, or pharmaceutically acceptable salt thereof, according to any one of the previous embodiments, wherein peptide fragment F2 comprises SEQ ID NO. 14 (VMTFDLTKITKTSSSFEVR).
18—The peptide, or pharmaceutically acceptable salt thereof, according to any one of the previous embodiments wherein the peptide fragment F2 consists of SEQ ID NO. 12 (SFEVR).
19—The peptide, or pharmaceutically acceptable salt thereof, according to any one of embodiments 1-14 wherein the peptide fragment F2 consists of SEQ ID NO. 13 (TKTSSSFEVR).
20—The peptide, or pharmaceutically acceptable salt thereof, according to any one of embodiments 1-17 wherein the peptide fragment F2 consists of SEQ ID NO. 14 (VMTFDLTKITKTSSSFEVR).
21—The peptide, or pharmaceutically acceptable salt thereof, according to anyone of embodiments 1-14, wherein the peptide fragment F2 has SEQ ID NO. 91, particularly, wherein the peptide fragment F2 has a sequence selected from the group consisting of SEQ ID NO. 13, 92-106.
22—The peptide, or pharmaceutically acceptable salt thereof, according to any one of the previous embodiments, wherein the peptide fragment F3 comprises SEQ ID NO. 15 (GDTNPKDDWFMLGLRDGRPE).
23—The peptide, or pharmaceutically acceptable salt thereof, according to any one of the previous embodiments, wherein the peptide fragment F3 consists of SEQ ID NO. 4 (GDTNPKDDWF).
24—The peptide, or pharmaceutically acceptable salt thereof, according to any one of the previous embodiments, wherein the peptide fragment F3 consists of SEQ ID NO. 15 (GDTNPKDDWFMLGLRDGRPE).
25—The peptide, or pharmaceutically acceptable salt thereof, according to any one of the previous embodiments wherein the peptide fragment F1 is SEQ ID NO. 9 (TWDPEGVIFY), the peptide fragment F2 is SEQ ID NO. 13 (TKTSSSFEVR) and/or the peptide fragment F3 is SEQ ID NO. 4 (GDTNPKDDWF).
26—The peptide, or pharmaceutically acceptable salt thereof according to any one of the previous embodiments that does not comprise a peptide fragment F3.
27—The peptide, or pharmaceutically acceptable salt thereof according to any one of the previous embodiments that has sequence SEQ ID NO. 16 (TKTSSSFEVRTWDPEGVIFY), SEQ ID NO. 17 (TWDPEGVIFYGDTNPKDDWF), SEQ ID NO. 18 (TKTSSSFEVRTWDPEGVIFYGDTNPKDDWF), SEQ ID NO. 19 (TWDPEGVIFYGDTNPKDDWFMLGLRDGRPE), or SEQ ID NO. 20 (VMTFDLTKITKTSSSFEVRTWDPEGVIFY).
28—The peptide, or pharmaceutically acceptable salt thereof, according to any one of the previous embodiments that has sequence SEQ ID NO. 16 (TKTSSSFEVRTWDPEGVIFY).
29—The peptide, or pharmaceutically acceptable salt thereof according to any one of the previous embodiments, wherein at least one amino acid of the peptide is a D-amino acid.
30—The peptide, or pharmaceutically acceptable salt thereof according to any one of the previous embodiments, wherein at least one amino acid of the peptide fragment F1 is a D-amino acid.
31—The peptide, or pharmaceutically acceptable salt thereof according to any one of the previous embodiments, wherein at least one amino acid of the peptide fragment F2 is a D-amino acid.
32—The peptide, or pharmaceutically acceptable salt thereof according to any one of the previous embodiments, wherein at least one amino acid of the peptide fragment F3 is a D-amino acid.
33—The peptide, or pharmaceutically acceptable salt thereof according to any one of the previous embodiments, wherein all the amino acids of the peptide fragment F1 are D-amino acids.
34—The peptide, or pharmaceutically acceptable salt thereof according to any one of the previous embodiments, wherein all the amino acids of the peptide fragment F2 are D-amino acids.
35—The peptide, or pharmaceutically acceptable salt thereof according to any one of the previous embodiments, wherein all the amino acids of the peptide fragment F3 are D-amino acids.
36—The peptide, or pharmaceutically acceptable salt thereof according to any one of the previous embodiments, wherein all the amino acids of the peptide are D-amino acids.
37—The peptide, or pharmaceutically acceptable salt thereof according to any one of the previous embodiments, wherein:
   (i) the N-terminal end of the peptide corresponds to —NR₁R₂, being R₁ and R₂ independently selected from the group consisting of —H, —C(O)R₃, a triglyceride, (C₁-C₂₀)alkyl and (C₁-C₂₀)alkenyl and R₃ is —H, (C₁-C₂₀)alkyl or (C₁-C₂₀)alkenyl; and
   (ii) the C-terminal end of the peptide corresponds to —COOR₄ or —C(O)NR₅R₆ wherein R₄ to R₆ are radicals independently selected from the group consisting of: —H and (C₁-C₂₀)alkyl.
38—The peptide, or pharmaceutically acceptable salt thereof according to any one of the previous embodiments, wherein the N-terminal end is —NH₂
39—The peptide, or pharmaceutically acceptable salt thereof according to any one of the previous embodiments, wherein the N-terminal end is —NHC(O)R₃, being R₃ a (C₁-C₂₀)alkyl or (C₁-C₂₀)alkenyl.
40—The peptide, or pharmaceutically acceptable salt thereof according to any one of the previous embodiments, wherein the N-terminal end is —NHC(O)R₃, being R₃ a (C₁-C₁₅)alkyl or (C₁-C₁₅)alkenyl.
41—The peptide, or pharmaceutically acceptable salt thereof according to any one of the previous embodiments, wherein the N-terminal end is —NHC(O)CH₃.
42—The peptide, or pharmaceutically acceptable salt thereof according to any one of the previous embodiments, wherein the C-terminal end is —COOH, —C(O)NH₂ or —C(O)NHR₆, wherein R₆ is (C₁-C₂₀)alkyl.
43—The peptide, or pharmaceutically acceptable salt thereof according to any one of the previous embodiments, wherein the C-terminal end is —COOH.
44—The peptide, or pharmaceutically acceptable salt thereof according to any one of the previous embodiments, wherein the C-terminal end is —C(O)NH₂
45—The peptide, or pharmaceutically acceptable salt thereof according to any one of the previous embodiments, wherein the C-terminal end is —C(O)NHR₆, wherein R₆ is (C₁-C_{2K})alkyl.
46—The peptide, or pharmaceutically acceptable salt thereof according to any one of the previous embodiments, wherein the C-terminal end is —C(O)NHCH₃.
47—The peptide, or pharmaceutically acceptable salt thereof according to any one of the previous embodiments, wherein the peptide, or pharmaceutically acceptable salt thereof has lipolytic activity.
48—The peptide, or pharmaceutically acceptable salt thereof according to any one of the previous embodiments, wherein the peptide, or pharmaceutically acceptable salt thereof has anti-lipogenic activity.
49—The peptide, or pharmaceutically acceptable salt thereof according to any one of the previous embodiments, wherein the peptide, or pharmaceutically acceptable salt thereof has antifibrotic activity.
50—The peptide, or pharmaceutically acceptable salt thereof according to any one of the previous embodiments, wherein the peptide, or pharmaceutically acceptable salt thereof has an anti-inflammatory activity.
51—A pharmaceutical composition comprising a therapeutical effective amount of a peptide, or pharmaceutically acceptable salt thereof, as defined in any one of the previous embodiments and one or more therapeutically acceptable carriers.
52—The peptide, or pharmaceutically acceptable salt thereof as defined in any one of embodiments 1-50, or the pharmaceutical composition as defined in embodiment 51, for use in therapy.
53—The peptide, or pharmaceutically acceptable salt thereof according to any one of embodiments 1-50, or the pharmaceutical composition according to embodiments 51, for use in the treatment and/or prevention of a condition or disease caused or associated to the accumulation of fat in an organ or tissue, inflammation and/or fibrosis.
54—The peptide, or pharmaceutically acceptable salt thereof, or the pharmaceutical composition for use according to embodiment 53, wherein the condition or disease is selected from the group consisting of non-alcoholic steatohepatitis (NASH), hepatic fibrosis, cirrhosis, hepatic steatosis, overweight, obesity, inflammatory disease, lipohyperdystrophy, diabetes and cardiovascular disease.
55—The peptide, or pharmaceutically acceptable salt thereof, or the pharmaceutical composition for use according to any one of embodiments 53-54, wherein the disease is non-alcoholic steatohepatitis (NASH).
56—A cosmetic composition comprising the peptide, or pharmaceutically acceptable salt thereof, according to any one of embodiments 1-50 and one or more cosmetically acceptable carriers.
57—Use of the peptide or pharmaceutically acceptable salt thereof according to any one of embodiments 1-50 or the cosmetic composition according to embodiment 56, in cosmetics.
58—The use according to embodiment 57 for reducing the subcutaneous fat in a mammal, preferably accumulated within the fibrous connective tissue under the skin of the mammal.
59—The use according to any one of embodiments 57 or 58 for reducing cellulite in a mammal.
60—A kit of parts comprising:
   a peptide or pharmaceutically acceptable salt thereof as defined in any one of embodiments 1-50, the pharmaceutical composition according to embodiment 51, or the cosmetic composition according to embodiment 56, and
   a device selected from the group consisting of an injection device, an implant, or a biodegradable container.
61—A device, such as an injection device, an implant, or a biodegradable container, comprising the peptide or pharmaceutically acceptable salt thereof as defined in any one of embodiments 1-50, the pharmaceutical composition as defined in embodiment 51 or the cosmetic composition as defined in embodiment 56.

### EXAMPLES

### Example 1. Synthesis of the Peptides of the Invention

### Synthesis:

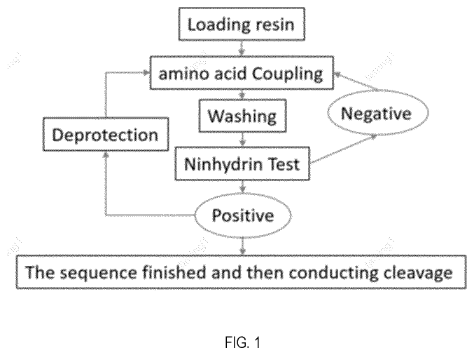

The method for peptide production is solid phase peptide synthesis (SPPS). The Fmoc chemistry is always used. SPPS is to sequentially add amino acids to the resin to build a peptide chain. When the synthesis is finished, the Fmoc group at N-terminal is first de-protected, and then, the side chain protection group de-protected, and the peptide is cleaved off from the Resin. The specific procedure is as follows (see FIG. 1):

### 1. Coupling the First Amino Acid.

Take the appropriate amount of modified Chloride resin, add the amino acids solution which were prepared in advance into the resin and react for a period time.

### 2. Removing the Fmoc

Add pip/DMF solution for a period time and then remove the solvent via vacuum filtration.

3. Washing after Removing the Fmoc

Add DMF to the resin (washing step), and then remove the solvent via vacuum filtration.

### 4. Detecting the Resin

Detection reagent and a spot of resin were taken into one test tube. And then the test tube was put into high temperature water for seconds to check if the color of the resin was changed. If the color of the resin changed, the Fmoc groups were removed successfully.

### 5. Coupling

Add the amino acid solution which were prepared in advance into the resin. And then add coupling reagent and shake for a period time, and then remove the solvent by vacuum filtration.

### 6. Coupling/Deprotection Cycle

Repeat Step 2-5, until the last amino acid synthesis is finished.

### Cleavage:

Adding proper amount of cleavage reagents, the peptide is cleaved off from the Resin.

### Purification:

### HPLC:

1 MS detection shows the product is correct
2: dissolve the peptide sample (0.5 mg), use HPLC (10-100% gradient) to get the real time of peak.

TABLE 1

| Column: | Kromasil C18, 4.6*250 mm, 5 μm | | |
|---|---|---|---|
| Solvent A | A: 0.1% Trifluoroacetic Acid in 100% Acetonitrile | | |
| Solvent B | B: 0.1% Trifluoroacetic Acid in 100% Water | | |
| Gradient: | | A | B |
| | 0.0 min | 10% | 90% |
| | 25.0 min | 100% | 0% |
| | 25.1 min | 100% | 0% |

| | |
|---|---|
| 30.0 min | Stop |

| | |
|---|---|
| Volume: | 20 μl |
| Wavelength | 220 nm |
| Flow rate: | 1.0 ml/min |

3 Make the gradient accordingly (ACN:H₂O=1:7 gradient=12-22%)

TABLE 2

| Column: | Hanbang DAC-HB100 | | |
|---|---|---|---|
| Solvent A | A: 0.1% Trifluoroacetic Acid in 100% Acetonitrile | | |
| Solvent B | B: 0.1% Trifluoroacetic Acid in 100% Water | | |
| Gradient: | | A | B |
| | 0.0 min | 12% | 88% |
| | 25.0 min | 22% | 78% |
| | 25.1 min | 100% | 0% |

| | |
|---|---|
| 30.0 min | Stop |

| | |
|---|---|
| Volume: | 300 ml |
| Wavelengt: | 220 nm |
| Flow rate: | 180.0 ml/min |

4. Purity detection: collect correct sample according to the detection

TABLE 3

| Column: | Kromasil C18, 4.6*250 mm, 5 μm | | |
|---|---|---|---|
| Solvent A | A: 0.1% Trifluoroacetic Acid in 100% Acetonitrile | | |
| Solvent B | B: 0.1% Trifluoroacetic Acid in 100% Water | | |
| Gradient: | | A | B |
| | 0.0 min | 15% | 85% |
| | 25.0 min | 40% | 60% |
| | 25.1 min | 100% | 0% |

| | |
|---|---|
| 30.0 min | Stop |

| | |
|---|---|
| Volume: | 20 μl |
| Wavelength: | 220 nm |
| Flow rate: | 1.0 ml/min |

5: rotary evaporate the collected correct product
6: lyophilization at −50° C.
7: Retest using HPLC to test the final purity of the dried product.

TABLE 4

| Column: | Kromasil C18, 4.6*250 mm, 5 μm | | |
|---|---|---|---|
| Solvent A | A: 0.1% Trifluoroacetic Acid in 100% Acetonitrile | | |
| Solvent B | B: 0.1% Trifluoroacetic Acid in 100% Water | | |
| Gradient: | | A | B |
| | 0.0 min | 15% | 85% |
| | 25.0 min | 40% | 60% |
| | 25.1 min | 100% | 0% |

| | |
|---|---|
| 30.0 min | Stop |

| | |
|---|---|
| Volume: | 20 μl |
| Wavelength | 220 nm |
| Flow rate: | 1.0 ml/min |

### MS:

1: take crude product 0.1 mg, dissolve it into ACN:H2O=2:1(looks transparent)
2: Wash the product using B-2060 sonic washer (30 seconds)
3: Centrifuge using RJ-TGL-16B centrifugal machine (1 min)
4. inject 0.5 ul into LCMS-2010EV MS detector to test the peak.

The characterization data of the peptides are provided in table 5 bellow.

### Fmoc-SolidPhase Synthesis Route for GP6: TKTSSSFEVRTWDPEGVIFY (all D Amino Acids)

1. Swelled the Fmoc-D-Tyr(tbu)-Resin in DCM for 30 mins
2. Washed with DMF three times; DBLK deprotection treatment for 15 mins; Washed with DMF six times
3. Coupled 5.8 g of Fmoc-D-Phe-OH, reacted for 30 mins
4. Tested qualified (solution was bright yellow, and resin transparent); repeated step 2
5. Coupled 5.3 g of Fmoc-D-Ile-OH, 5.1 g of Fmoc-D-Val-OH, 4.5 g of Fmoc-Gly-OH, 6.4 g of, Fmoc-D-Glu(otbu)-OH, 5.1 g of Fmoc-D-Pro-OH, 6.2 g of Fmoc-D-Asp(otbu)-OH, 7.9 g of Fmoc-D-Trp(boc)-OH, 6.0 g of Fmoc-D-Thr(tbu)-OH, 9.8 g of Fmoc-D-Arg(pbf)-OH, 5.1 g of Fmoc-D-Val-OH, 6.4 g of Fmoc-D-Glu(otbu)-OH, 5.8 g of Fmoc-D-Phe-OH, 5.8 g of Fmoc-D-Ser(tbu)-OH, 5.8 g of Fmoc-D-Ser(tbu)-OH, 5.8 g of Fmoc-D-Ser(tbu)-OH, 6.0 g of Fmoc-D-Thr(tbu)-OH, 7.0 g of Fmoc-D-Lys(boc)-OH, sequentially, each reaction allowed 30 mins and followed with step 2
6. Coupled the last AA, 6.0 g of Fmoc-D-Thr(tbu)-OH, reacted for 30 mins, Tested qualified, Washed with DMF three times; DBLK deprotection treatment for 15 mins;
7. Washed with DMF, DCM and MeOH separately, each three times, then cleaved, and followed with purification and lyophilization.

### Fmoc-SolidPhase Synthesis Route for GP8: TKTSSSFEVRTWDPEGVIFYGDTNPKDDWF (all D Amino Acids)

1. Swelled the Fmoc-D-Phe-Resin in DCM for 30 mins
2. Washed with DMF three times; DBLK deprotection treatment for 15 mins; Washed with DMF six times
3. Coupled 0.18 g of Fmoc-D-Trp(Boc)-OH, reacted for 30 mins
4. Tested qualified (solution was bright yellow, and resin transparent); repeated step 2
5. Coupled 0.14 g of Fmoc-D-Asp(OtBu)-OH, 0.14 g of Fmoc-D-Asp(OtBu)-OH, 0.16 g of Fmoc-D-Lys(Boc)-OH, 0.12 g of, Fmoc-D-Pro-OH, 0.21 g of Fmoc-D-Asn(Trt)-OH, 0.14 g of Fmoc-D-Thr(tbu)-OH, 0.14 g of Fmoc-D-Asp(OtBu)-OH, 0.10 g of Fmoc-Gly-OH, 0.16 g of Fmoc-D-Tyr(tBu)-OH, 0.14 g of Fmoc-D-Phe-OH, 0.12 g of Fmoc-D-Allo-Ile-OH, 0.12 g of Fmoc-D-Val-OH, 0.10 g of Fmoc-Gly-OH, 0.15 g of Fmoc-D-Glu(OtBu)-OH, 0.12 g of Fmoc-D-Pro-OH, 0.14 g of Fmoc-D-Asp(OtBu)-OH, 0.18 g of Fmoc-D-Trp(Boc)-OH, 0.14 g of Fmoc-D-Thr(tBu)-OH, 0.23 g of Fmoc-D-Arg(Pbf)-OH, 0.12 g of Fmoc-D-Val-OH, 0.15 g of Fmoc-D-Glu(OtBu)-OH, 0.14 g of Fmoc-D-Phe-OH, 0.13 g of Fmoc-D-Ser(tbu)-OH, 0.13 g of Fmoc-D-Ser(tbu)-OH, 0.13 g of Fmoc-D-Ser(tbu)-OH, 0.14 g of Fmoc-D-Thr(tbu)-OH, 0.16 g of Fmoc-D-Lys(Boc)-OH, sequentially, each reaction allowed 30 mins and followed with step 2
6. Coupled the last AA, 0.14 g of Fmoc-D-Thr(tbu)-OH, reacted for 30 mins, Tested qualified, Washed with DMF three times; DBLK deprotection treatment for 15 mins;
7. Washed with DMF, DCM and MeOH separately, each three times, then cleaved, and followed with purification and lyophilization.

Peptides other than GP8 where synthesized following the synthesis route indicated above for peptide GP6. The synthesized peptides are provided in table 5 bellow.

| SEQ | Product | | | | | Nr. | HPLC | |
|---|---|---|---|---|---|---|---|---|
| ID | Name | Peptide Sequence (1 letter code) | N-term | C-Term | Modif. Seq | AA | purity | MS |
| 20 | GP4 | VMTFDLTKITKTSSSFEVRTWDPEGVIFY | NH₂ | COOH | (I = D-Ile; All D amino acid) | 29 | 96.08% | 3398.83 |
| 16 | GP6 | TKTSSSFEVRTWDPEGVIFY | NH₂ | COOH | (I = D-Ile; All D amino acid) | 20 | 95.84% | 2349.75 |
| 17 | GP7 | TWDPEGVIFYGDTNPKDDWF | NH₂ | COOH | (I = D-Ile; All D amino acid) | 20 | 96.01% | 2402.58 |
| 18 | GP8 | TKTSSSFEVRTWDPEGVIFYGDTNPKDDWF | NH₂ | COOH | (I = D-Ile; All D amino acid) | 30 | 95.44% | 3525.74 |
| 19 | GP9 | TWDPEGVIFYGDTNPKDDWFMLGLRDGRPE | NH₂ | COOH | (I = D-Ile; All D amino acid) | 30 | 95.18% | 3527.9 |

| SEQ | | | | | Nr. |
|---|---|---|---|---|---|
| ID | Peptide Sequence (1 letter code) | N-term | C-Term | Modif. Seq | AA |
| 127 | Ac-TKTSSSFEVRTWDPEGVIFY | CH3CONH | COOH | (I = D-Ile; All D amino acid) | 20 |
| 128 | Palmitoyl-TKTSSSFEVRTWDPEGVIFY | CH3(CH2)14CONH | COOH | (I = D-Ile; All D amino acid) | 20 |
| 129 | TKTSSSFEVRTWDPEGVIFY-CONH2 | NH2 | CONH2 | (I = D-Ile; All D amino acid) | 20 |
| 130 | TKTSSSFEVRTWDPEGVIFY-CONHCH3 | NH2 | CONHCH3 | (I = D-Ile; All D amino acid) | 20 |
| 131 | TKTSSSFEVRTWNPEGVIFY | NH2 | COOH | (I = D-Ile; All D amino acid) | 20 |
| 132 | TKTSSSFEVRTWDPQGVIFY | NH2 | COOH | (I = D-Ile; All D amino acid) | 20 |
| 133 | TKTSSSFEVRTNDPEGVIFY | NH2 | COOH | (I = D-Ile; All D amino acid) | 20 |
| 134 | TKTSSSFQVRTWDPEGVIFY | NH2 | COOH | (I = D-Ile; All D amino acid) | 20 |
| 135 | T(Ac-)KTSSSFEVRTWDPEGVIFY | NH2 | COOH | (I = D-Ile; All D amino acid) | 20 |
| 136 | TKTSSSFEVRT(Ac-)WDPEGVIFY | NH2 | COOH | (I = D-Ile; All D amino acid) | 20 |
| 137 | T(Palmitoyl-)KTSSSFEVRTWDPEGVIFY | NH2 | COOH | (I = D-Ile; All D amino acid) | 20 |
| 138 | TKTSSSFEVRT(Palmitoyl-)WDPEGVIFY | NH2 | COOH | (I = D-Ile; All D amino acid) | 20 |
| 139 | TKTSSSFEVRTWDPEGVLFY | NH2 | COOH | (All D amino acid) | 20 |
| 140 | KTSSSFEVRTWDPEGVIFY | NH2 | COOH | (I = D-Ile; All D amino acid) | 19 |
| 141 | TSSSFEVRTWDPEGVIFY | NH2 | COOH | (I = D-Ile; All D amino acid) | 18 |
| 142 | SSSFEVRTWDPEGVIFY | NH2 | COOH | (I = D-Ile; All D amino acid) | 17 |
| 143 | SSFEVRTWDPEGVIFY | NH2 | COOH | (I = D-Ile; All D amino acid) | 16 |
| 144 | SFEVRTWDPEGVIFY | NH2 | COOH | (I = D-Ile; All D amino acid) | 15 |
| 145 | AKTSSSFEVRTWDPEGVIFY | NH2 | COOH | (I = D-Ile; All D amino acid) | 20 |
| 146 | TATSSSFEVRTWDPEGVIFY | NH2 | COOH | (I = D-Ile; All D amino acid) | 20 |
| 147 | TKASSSFEVRTWDPEGVIFY | NH2 | COOH | (I = D-Ile; All D amino acid) | 20 |
| 148 | TKTASSFEVRTWDPEGVIFY | NH2 | COOH | (I = D-Ile; All D amino acid) | 20 |
| 149 | TKTSASFEVRTWDPEGVIFY | NH2 | COOH | (I = D-Ile; All D amino acid) | 20 |
| 150 | TKTSSSFEVRTWDPEGVLFY | NH2 | COOH | (All D amino acid) | 20 |
| 151 | TKTSSSFEVRITIWIDIPIEIGIVILIFIY | NH2 | COOH | (All L amino acid) | 20 |
| 152 | TKTSSSFEVRAWDPEGVLFY | NH2 | COOH | (All D amino acid) | 20 |
| 153 | TKTSSSFEVRTADPEGVLFY | NH2 | COOH | (All D amino acid) | 20 |
| 154 | TKTSSSFEVRTNDPEGVLFY | NH2 | COOH | (All D amino acid) | 20 |
| 155 | TKTSSSFEVRAWDPEGVLFY | NH2 | COOH | (All D amino acid) | 20 |
| 156 | TKTSSSFEVRANDPEGVLFY | NH2 | COOH | (All D amino acid) | 20 |
| 157 | TKTSSSFEVRNDPEGVLFY | NH2 | COOH | (All D amino acid) | 19 |
| 158 | TKTSSSFEVRNDEGLFY | NH2 | COOH | (All D amino acid) | 17 |
| 159 | TKTSSSFEVRNDPEGLY | NH2 | COOH | (All D amino acid) | 17 |
| 160 | TKTSSSFEVRNDLEGLY | NH2 | COOH | (All D amino acid) | 17 |
| 161 | TKTSSSFEVRINIDILIEIGILIY | NH2 | COOH | (All L amino acid) | 17 |

TABLE 6

| SEQ ID NO. | Peptide sequence (1 letter code) | Modif. Seq. |
|---|---|---|
| 23 | TWDPEGVIFY | (I = D-Ile; All D amino acid) |
| 37 | TDPEGVIY | (i = Alo-Ile, All D aa) |
| 38 | TDPEGVIF | (i = Alo-Ile, All D aa) |
| 35 | WDPEGVIY | (i = Alo-Ile, All D aa) |
| 36 | WDPEGVIF | (i = Alo-Ile, All D aa) |
| 39 | TWDPEGVI | (i = Alo-Ile, All D aa) |
| 24 | WDPEGVIFY | (i = Alo-Ile, All D aa) |
| 25 | TDPEGVIF | (i = Alo-Ile, All D aa) |
| 26 | TWPEGVIFY | (i = Alo-Ile, All D aa) |
| 27 | TWDEGVIFY | (i = Alo-Ile, All D aa) |
| 32 | TWDPEGVIY | (i = Alo-Ile, All D aa) |
| 28 | TWDPGVIFY | (i = Alo-Ile, All D aa) |
| 29 | TWDPEVIFY | (i = Alo-Ile, All D aa) |
| 30 | TWDPEGIFY | (i = Alo-Ile, All D aa) |
| 31 | TWDPEGVFY | (i = Alo-Ile, All D aa) |
| 33 | TWDPEGVIF | (i = Alo-Ile, All D aa) |
| 46 | DPEGVI | (I = D-Ile; All D aa) |
| 41 | DPEGVIY | (I = D-Ile; All D aa) |
| 40 | DPEGVFY | (I = D-Ile; All D aa) |
| 42 | DPEGVIF | (I = D-Ile; All D aa) |
| 34 | DPEGVIFY | (I = D-Ile; All D aa) |
| 49 | WDPEG | (All D amino acid) |
| 47 | WDPEGV | (All D amino acid) |
| 48 | TWDPEG | (All D amino acid) |
| 43 | WDPEGVI | (I = D-Ile; All D amino acid) |
| 44 | WDPEGVY | (All D amino acid) |
| 45 | WDPEGVF | (All D amino acid) |
| 50 | WDPEGLY | (All D amino acid) |
| 51 | DPEGLFY | (All D amino acid) |
| 52 | DPEGVLY | (All D amino acid) |
| 53 | WDNEGLY | (All D amino acid) |
| 54 | NDPEGLY | (All D amino acid) |
| 55 | NDPEGVIY | (I = D-Ile; All D amino acid) |
| 67 | NDVEGLY | (All D amino acid) |
| 68 | NDAEGLY | (All D amino acid) |
| 69 | NDLEGVY | (All D amino acid) |
| 70 | NDLEGAY | (All D amino acid) |
| 71 | NDLEGPY | (All D amino acid) |
| 57 | LN-LD-LP-LE-LG-LL-LY | (All L amino acid) |
| 58 | DN-LD-LP-LE-G-LL-LY | L,D-amino acid scan |
| 59 | LN-DD-LP-LE-G-LL-LY | L,D-amino acid scan |
| 60 | LN-LD-DP-LE-G-LL-LY | L,D-amino acid scan |
| 61 | LN-LD-LP-DE-G-LL-LY | L,D-amino acid scan |
| 62 | LN-LD-LP-LE-G-DL-LY | L,D-amino acid scan |
| 63 | LN-LD-LP-LE-G-LL-DY | L,D-amino acid scan |
| 72 | NDEGLFY | (All D amino acid) |
| 56 | NDPEGY | (All D amino acid) |
| 73 | NDLEGLY | (All D amino acid) |
| 74 | NDEGLY | (All D amino acid) |
| 75 | NDEGLY | (All D amino acid) |
| 76 | NDEGLY | (All D amino acid) |
| 78 | TWPEGVIFY | each amnino acid L- or D-aa |
| 79 | TWDEGVIFY | each amnino acid L- or D-aa |
| 80 | TWDPGVIFY | each amnino acid L- or D-aa |
| 81 | TWDPEVIFY | each amnino acid L- or D-aa |
| 82 | TWDPEGVIFY | each amino acid L- or D-aa provided |
| | | at least one is D-aa, includes |
| | | amidation in each amino acid |

### Example 2. Effects of Peptides GP4. GP7. GP8 and GP9 (Peptides with SEQ ID NO. 20. SEQ ID NO. 17. SEQ ID NO. 18. SEQ ID NO. 19, Respectively) in Hepatic Lipogenesis in HepG2 Cells, Fibrosis in LX-2 Cells and in Lipolysis in Human Mature Adipocytes

### Material and Methods

### Cell Culture Experiments

Cell culture reagents were from Life Technologies Inc (Invitrogen SA, Barcelona, Spain). HepG2 hepatoblastoma cells (cat. no. HB-8065; ATCC, LGC Standards SLU, Barcelona, Spain) were maintained in Dulbecco's modified Eagle medium (DMEM) supplemented with 10% fetal bovine serum and antibiotics. HepG2 cells were treated with vehicle or 10 μM of GP4, GP7, GP8 and GP9 for 24 h. LX-2 human hepatic stellate cells (SCC064) were maintained in DMEM supplemented with 2% fetal bovine serum and antibiotics. For experiments, LX-2 were cultured for 24 h in DMEM 0.2% FBS and treated for 24 h with vehicle or TGFβ1 (20 ng/ml) in the absence or presence of 8 μM of GP4, GP7, GP8 and GP9 for 24 h. At the end of the experiment's cells were harvested for gene expression analysis.

Human preadipocytes and cell culture reagents were from purchased from Tebu-Bio (Tebu-bio, Barcelona, Spain) and differentiation protocol was done following the manufacturer's instructions. Human adipocytes were maintained in a humidified incubator with 5% CO2 at 37° C. (Sex hormone-binding globulin overexpression protects against high-fat diet-induced obesity in transgenic male mice. Saez-Lopez C, Villena J A, Simo R, Selva D M. J Nutr Biochem. 2020 November; 85:108480. doi: 10.1016/j.jnutbio.2020.108480).

### Lipolysis Assay

Lipolysis assays were performed using a Lipolysis Kit (Tebu-Bio) following the manufacturer's instructions as previously described.

### RNA Analysis

Total RNA was extracted from HepG2 and LX-2 cells using TRIzol reagent (Invitrogen SA). Reverse transcription was performed at 42° C. for 50 minutes using 3 mg of total RNA, 200 U of Superscript II, and an oligo-dT primer and reagents provided by Invitrogen. An aliquot of the reverse transcription product was amplified in a 25-mL reaction using SYBRGreen (Invitrogen SA) with appropriate oligonucleotide primer pairs corresponding to human ACC, FAS, ACLY, Col1A1 and 18S (shown in table 7). Results were analyzed using the 7000 Sequence Detection System program (Applied Biosystems, Thermo Fisher Scientific, Barcelona, Spain) (Sex Hormone-Binding Globulin Reduction in Metabolic Disorders May Play a Role in NAFLD Development.Saez-Lopez C, Barbosa-Desongles A, Hernandez C, Dyer R A, Innis S M, Simo R, Selva D M. Endocrinology. 2017 Mar. 1; 158(3):545-559. doi: 10.1210/en.2016-1668).

All the results are shown as Mean±SEM in the figures. One-way ANOVA and Bonferroni's post-hoc test compared to Control and/or Vehicle group were performed depending on the experiment. * p<0.05 and **p<0.01

### Results

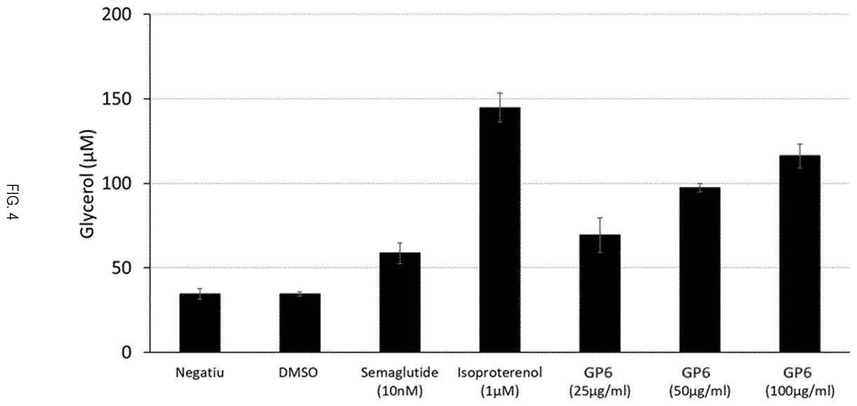

Effects of GP4, GP7, GP8 and GP9 in hepatic lipogenesis in HepG2 cells GP4, GP5, GP8 and GP9 showed different activities in regulating key hepatic lipogenic enzymes such as Acetyl-CoA Carboxylase (ACC), Fatty Acid Synthase (FAS) and ATP-Citrate Lyase (ACLY) in HepG2 cells (FIG. 5). GP4 treatment did not alter ACLY, reduced ACC and increased FAS mRNA levels. GP7 treatment reduced ACC and ACLY but increased FAS mRNA levels. GP8 reduced ACC, FAS and ACLY mRNA levels and GP9 did not alter ACC and ACLY but reduced FAS mRNA levels (FIG. 5). Therefore, all four peptides (GP4, GP7, GP8 and GP9) could be used potentially to reduce lipogenesis in the liver since they all reduce the expression of at least one of the three key enzymes of hepatic lipogenesis.

### Effects of GP8 and GP9 in Fibrosis in LX-2 Cells

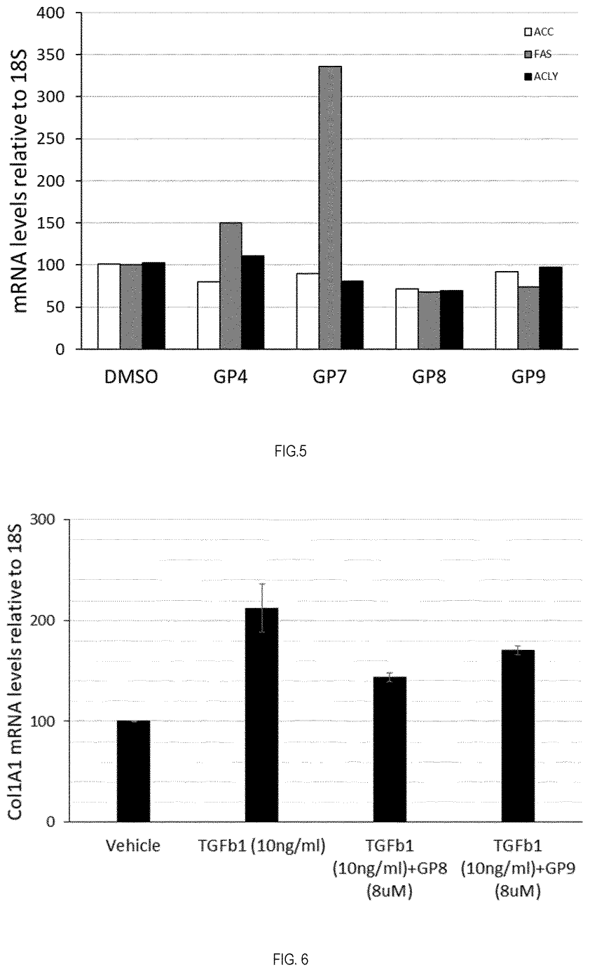

GP8 and GP9 showed different activities in regulating collagen 1A1 expression induced by TGFβ1 in human hepatic stellate cell line (LX-2). Col1A1 mRNA levels were reduced by treatment with GP8 (31%) and GP9 (21%) (FIG. 6).

### Effects of GP4, GP8 and GP9 in Lipolysis in Human Mature Adipocytes

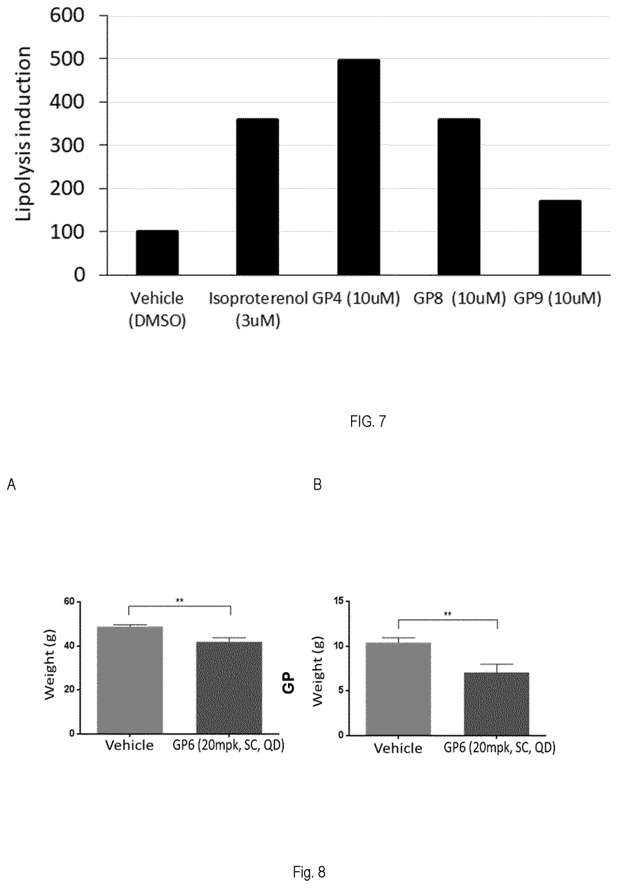

GP4, GP8 and GP9 treatment induced lipolysis measured by glycerol production after 16 h treatment in human mature adipocytes (FIG. 7). The lipolysis positive control (isoproterenol) increased glycerol concentration in a 370% with respect with the control while GP4 was able to increase glycerol production by 500%, GP8 by 370% and GP9 by 170% (FIG. 7) These in vitro assays with peptides GP4, GP7, GP8 and GP9 allow formulating the following conclusions:
We have designed several peptides that have activities in terms of inhibiting lipogenesis in hepatocytes, inhibiting fibrosis in hepatic stellate cells and stimulating lipolysis in human mature adipocytes.

### Example 3. Peptide GP6 (Peptide with SEQ ID NO. 16) Reduces Hepatic Lipogenesis in HepG2 Cells, Fibrosis in LX-2 Cells and Induces Lipolysis in Human Mature Adipocytes

### Materials and Methods

### Cell Culture Experiments

Cell culture reagents were from Life Technologies Inc (Invitrogen SA, Barcelona, Spain). HepG2 hepatoblastoma cells (cat. no. HB-8065; ATCC, LGC Standards SLU, Barcelona, Spain) were maintained in Dulbecco's modified Eagle medium (DMEM) supplemented with 10% fetal bovine serum and antibiotics. HepG2 cells were cultured for 5 day in the absence or presence of 30 mM fructose. Cells cultured in 30 mM fructose were daily treated with vehicle, Telmisartan, Semaglutide or GP6. LX-2 human hepatic stellate cells (SCC064) were maintained in DMEM supplemented with 2% fetal bovine serum and antibiotics. For experiments, LX-2 were cultured for 24 h in DMEM 0.2% FBS and treated for 24 h with vehicle or TGFβ1 (20 ng/ml) in the absence or presence of GP6 (10 or 40 μg/ml). At the end of the experiment's cells were harvested for gene expression analysis.

Human preadipocytes and cell culture reagents were from purchased from Tebu-Bio (Tebu-bio, Barcelona, Spain) and differentiation protocol was done following the manufacturer's instructions. Human adipocytes were maintained in a humidified incubator with 5% CO2 at 37° C. (Sex hormone-binding globulin overexpression protects against high-fat diet-induced obesity in transgenic male mice. Saez-Lopez C, Villena J A, Simo R, Selva D M. J Nutr Biochem. 2020 November; 85:108480. doi: 10.1016/j.jnutbio.2020.108480).

### Lipolysis Assay

Lipolysis assays were performed using a Lipolysis Kit (Tebu-Bio) following the manufacturer's instructions as previously described by Saez-Lopez and collaborators (Sex hormone-binding globulin overexpression protects against high-fat diet-induced obesity in transgenic male mice. Saez-Lopez C, Villena J A, Simo R, Selva D M. J Nutr Biochem. 2020 November; 85:108480. doi: 10.1016/j.jnutbio.2020.108480.)

### RNA Analysis

Total RNA was extracted from HepG2 and LX-2 cells using TRIzol reagent (Invitrogen SA). Reverse transcription was performed at 42° C. for 50 minutes using 3 mg of total RNA, 200 U of Superscript II, and an oligo-dT primer and reagents provided by Invitrogen. An aliquot of the reverse transcription product was amplified in a 25-mL reaction using SYBRGreen (Invitrogen SA) with appropriate oligonucleotide primer pairs corresponding to human ACC and PPARG, CoI1A1 and 18S (shown in table 7). Results were analyzed using the 7000 Sequence Detection System program (Applied Biosystems, Thermo Fisher Scientific, Barcelona, Spain) (Sex Hormone-Binding Globulin Reduction in Metabolic Disorders May Play a Role in NAFLD Development.Saez-Lopez C, Barbosa-Desongles A, Hernandez C, Dyer R A, Innis S M, Simo R, Selva D M. Endocrinology. 2017 Mar. 1; 158(3):545-559. doi: 10.1210/en.2016-1668).

TABLE. 7
Primers used in the examples.

| Gene | Forward | Reverse |
|---|---|---|
| ACCa | GCCATTGGTATTGGGGCTTAC | CCCGACCAAGGACTTTGTTG (SEQ |
| | (SEQ ID NO. 3) | ID NO. 21) |
| FAS | GGCATCATTGGGCACTCCTT (SEQ | GCTGCAAGCACAGCCTCTCT (SEQ |
| | ID NO. 22) | ID NO. 64 |
| ACLY | GAATGCTGGACAACATCCTG (SEQ | GTTCATTAGACATGCCTCCTG (SEQ |
| | ID NO. 65) | ID NO. 66) |
| PPARg | TCTTAACTGCCGGATCCACAA (SEQ | GCCCAAACCTGATGGCATT (SEQ |
| | ID NO. 77) | ID NO. 162) |
| TNFa | CATCTTCTCAAAATTCGAGTGACAA | TGGGAGTAGACAAGGTACAACCC |
| | (SEQ ID NO. 163) | (SEQ ID NO. 164) |
| TGFb | AGGGCTACCATGCCAACTTC (SEQ | CCACGTAGTAGACGATGGG (SEQ I |
| | ID NO. 165) | NO. 166) |
| IL6 | TCGTGGAAATGAGAAAAGAGTTG | AGTGCATCATCGTTGTTCATACA |
| | (SEQ ID NO. 167) | (SEQ ID NO. 168) |
| IL1b | CTGGTGTGTGACGTTCCCATTA | CCGACAGCACGAGGCTTT (SEQ ID |
| | (SEQ ID NO. 169) | NO. 170) |
| F4/80 | CTTTGGCTATGGGCTTCCAGTC | GCAAGGAGGACAGAGTTTATCGTG |
| | (SEQ ID NO. 171) | (SEQ ID NO. 172) |
| COL1A1 | GCTCCTCTTAGGGGCCACT (SEQ | ATTGGGGACCCTTAGGCCAT (SEQ |
| | ID NO. 173) | ID NO. 174) |
| Leptin | GAGACCCCTGTGTCGGTTC (SEQ | CTGCGTGTGTGAAATGTCATTG |
| | ID NO. 175) | (SEQ ID NO. 176) |
| Adipoq | TGTTCCTCTTAATCCTGCCCA (SEQ | CCAACCTGCACAAGTTCCCTT (SEQ |
| | ID NO. 177) | ID NO. 178) |
| 18s | AGGGTTCGATTCCGGAGAGG (SEQ | CAACTTTAATATACGCTATTGG |
| | ID NO. 125) | (SEQ ID NO. 126) |

All the results are shown as Mean±SEM in the figures. One-way ANOVA and Bonferroni's post-hoc test compared to Control and/or Vehicle group were performed depending on the experiment. * p<0.05 and **p<0.01.

### Results

### GP6 Reduces Hepatic Lipogenesis in HepG2 Cells

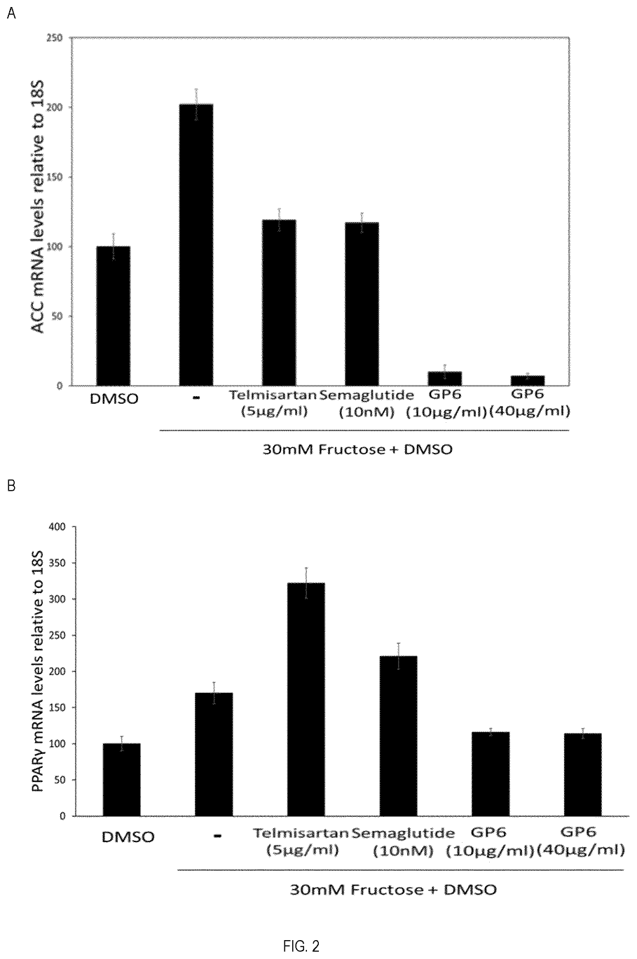

Daily treatment with GP6 reduced significantly hepatic lipogenesis induced by 30 mM fructose via reducing mRNA levels of a key hepatic lipogenic enzyme such as Acetyl-CoA Carboxylase (ACC) and a key transcription factor regulating several lipogenic enzymes such as PPARγ in HepG2 cells (FIG. 2). GP6 treatment was significantly much more effective than Telmisartan and Semaglutide, used as positive controls, in reducing the induced expression of ACC and PPARγ by 30 mM fructose treatment (FIG. 2).

### GP6 Reduces Fibrosis in LX-2 Cells

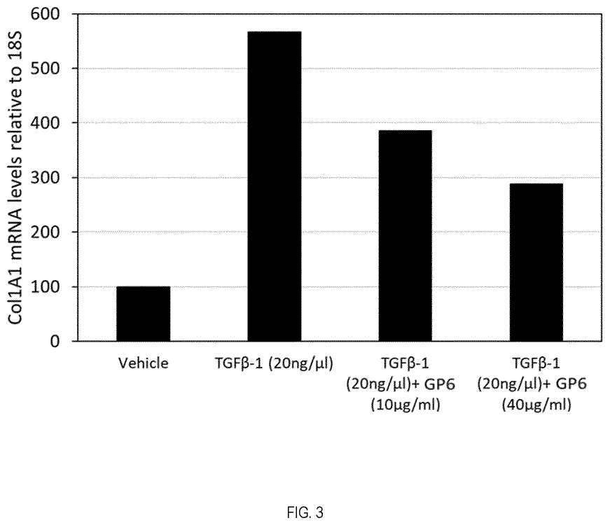

GP6 treatment reduced in 28% (10 μg/ml) and 45% (40 μg/ml) the mRNA levels of collagen 1A1 induced by TGFβ1 in human hepatic stellate cell line (LX-2) (FIG. 3).

GP6 induces lipolysis in human mature adipocytes GP6 induced lipolysis measured by glycerol production after 16 h treatment in human mature adipocytes (FIG. 4). The lipolysis positive control (isoproterenol) increased glycerol concentration in a 400% with respect with the controls while GP6 was able to increase glycerol production in a dose dependent manner by 200% (25 μg/ml), 270% (50 μg/ml) and 315% (100 μg/ml) performing more efficiently than Semaglutide (165% increase) (FIG. 4).

These in vitro assays with peptide GP6 allow formulating the following conclusions:
GP6 has anti-lipogenic activity by inhibiting the expression of ACC and PPARγ, two key players of hepatic lipogenesis in human liver cells (HepG2 cells).
GP6 has anti-fibrotic activity by inhibiting the expression of collagen 1A1 in human hepatic stellate cells (LX-2 cells).
GP6 has lipolytic activity by increasing lipolysis in human mature adipocytes.

### Example 4. Preventive Effects of Peptide GP6 (SEQ ID NO. 16) in an Obesity and Fatty Liver Disease Mouse Model

### Materials and Methods

### Animals

C57BL6/J male mice were purchased and kept for quarantine and acclimatization for a period of about 7 days before initiation of every experiment. Mice were housed in clean and sterilized mice cages and maintained in a controlled environment with 22+3° C. temperature, 50+20% humidity, a light/dark cycle of 12 hours each and 15-20 fresh air changes per hour.

### In Vivo Experiments

For Obesity and NAFLD induction, the animals were fed ad libitum, either with chow diet (150SP-10, Safe Diets) or 45% high-fat diet (TD.190179, Envigo) plus 30% fructose (F0127, Sigma Aldrich) in drinking water for 8 weeks in the daily presence of vehicle or a subcutaneous injection of GP6 (20 mg/kg/day). A group of mice (n=18) was randomly divided in two groups of 9 mice each. Both groups were fed with high fat diet and 30% fructose in the drinking water for 8 weeks to develop obesity and fatty liver disease. One group was treated with daily subcutaneous injections of vehicle (water) and the other with daily subcutaneous injection of GP6 (20 mg/kg/day) during the 8 weeks that last the experiment.

At the end of the experiment mice were sacrificed, serum samples were extracted, tissues were weighted and part was fixed in paraformaldehyde for histology studies and part immediately frozen at −80 C for mRNA and protein expression studies as well as assessment of triglycerides.

All experimental procedures were approved by the Institutional Animal Use Subcommittees of Vall Hebron University Hospital Research Institute and the Autonomous University of Barcelona.

### Histology

For histological studies, livers and adipose tissue were fixed in 4% paraformaldehyde for 24 hours and embedded in paraffin. Serial 5-mm-thick sections were used for histological examination and stained with hematoxylin eosin (H&E) and Picrosirius red for histological examination following standard procedures. Briefly, sections were alcohol-xylol hydrated at room temperature, and subsequently, they were stained at room temperature in a solution of 0.1% Direct Red 80 (Sigma-Aldrich) in a saturated aqueous solution of picric acid (Sigma-Aldrich) for 1 h. Following that, sections were rinsed twice in 0.5% acetic acid or 5 min, alcohol-xylol dehydrated and covered with Eukitt mounting medium (Sigma-Aldrich). These sections were examined with an Olympus BX61 microscope. Six random fields at 4× magnification from each slice were quantified using ImageJ software to determine the liver collagen deposition as the red stained area in the image. The stage of liver steatosis and/or fibrosis was assessed blinded by an experienced pathologist according to the NASH-clinical research network scoring system (Design and validation of a histological scoring system for nonalcoholic fatty liver disease. Kleiner D E, Brunt E M, Van Natta M, Behling C, Contos MJ, Cummings O W, Ferrell L D, Liu Y C, Torbenson M S, Unalp-Arida A, Yeh M, McCullough A J, Sanyal AJ; Nonalcoholic Steatohepatitis Clinical Research Network. Hepatology. 2005 June; 41(6):1313-21. doi: 10.1002/hep.20701; Transforming growth factor-beta 1: A new factor reducing hepatic SHBG production in liver fibrosis. Briansó-Llort L, Fuertes-Rioja L, Ramos-Perez L, Salcedo-Allende M T, Hernandez C, Simo R, Selva D M. J Cell Physiol. 2022 September; 237(9):3598-3613. doi: 10.1002/jcp.30818).

### Aspartate Transaminase (AST) and Alanine Transaminase (ALT) Measurements

Serum levels of AST and ALT were measured using MAK055 and MAK052 from Sigma-Aldrich respectively following the manufacture's instructions.

### Triglyceride (TG) Measurements

Hepatic TG content was measured from liver extracts using a Triglyceride Assay Kit (ab65336) following the manufacture's instructions.

### RNA Analysis

Total RNA was extracted from mouse tissues using TRIzol reagent (Invitrogen SA). Reverse transcription was performed at 42° C. for 50 minutes using 3 mg of total RNA, 200 U of Superscript II, and an oligo-dT primer and reagents provided by Invitrogen. An aliquot of the reverse transcription product was amplified in a 25-mL reaction using SYBRGreen (Invitrogen SA) with appropriate oligonucleotide primer pairs corresponding mouse ACC, FAS, ACLY, PPARG, Col1A1, F4/80, TNFα, IL1β, IL6, TGFβ1, Leptin, Adiponectin and 18S (shown in table 7). Results were analyzed using the 7000 Sequence Detection System program (Applied Biosystems, Thermo Fisher Scientific, Barcelona, Spain) (Sex Hormone-Binding Globulin Reduction in Metabolic Disorders May Play a Role in NAFLD Development.Saez-Lopez C, Barbosa-Desongles A, Hernandez C, Dyer R A, Innis S M, Simo R, Selva D M. Endocrinology. 2017 Mar. 1; 158(3):545-559. doi: 10.1210/en.2016-1668).

All the results are shown as Mean±SEM in the figures. One-way ANOVA and Bonferroni's post-hoc test compared to Control and/or Vehicle group were performed depending on the experiment. * p<0.05 and **p<0.01

### Results

### GP6 Treatment Reduces Total Body Weight and Body Weight

Total body weight and weight increment (weight at week 8-weight at week 0) was significantly reduced by daily subcutaneous treatment with GP6 (20 mg/kg/day) when compared with vehicle treated mice (FIG. 8).

### GP6 Treatment Reduces Total Liver and Adipose Tissue Weight

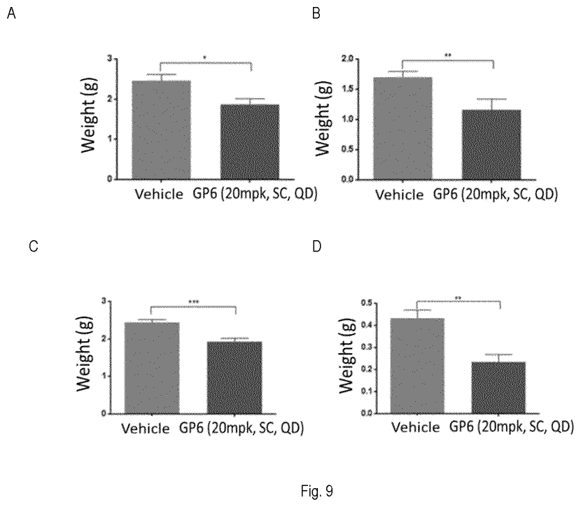

Daily subcutaneous treatment with GP6 (20 mg/kg/day) significantly reduced total liver weight and weight of subcutaneous and visceral adipose tissue as well as brown adipose tissue when compared with vehicle treated mice (FIG. 9).

### GP6 Treatment Reduces Hepatic Lipid Accumulation

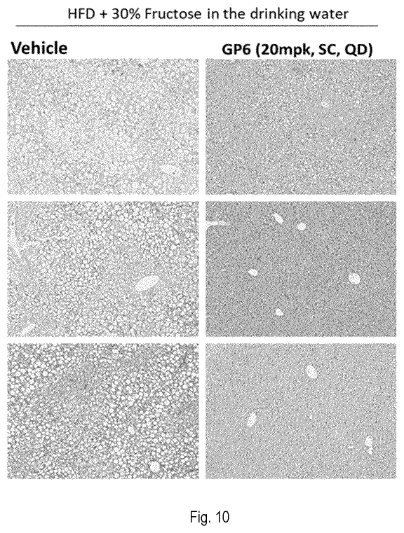

Histology studies showed that daily subcutaneous treatment with GP6 (20 mg/kg/day) reduced significantly hepatic lipid accumulation when compared with vehicle treated mice (FIG. 10).

The results obtained in the above histological studies were confirmed by measuring hepatic triglyceride content of the mice (Liver TAG) and by the blind analysis of an expert liver pathologist (Dr. Maria Teresa Salcedo Allende) from the Pathology Department of the Vall d'Hebron Hospital assessing the Steatosis and Brunt Scores [Brunt Scores include in all the examples herein the following parameters: Amount of fat (graded 1 to 3 according to the percentage of fatty droplets: 1 0%-33%; 2 34-66%; 3 67-100%). Fibrosis: graded 0 (absent) to 4 (1, perisinusoidal/pericellular fibrosis; 2, periportal fibrosis; 3, bridging fibrosis; 4, cirrhosis)] as well as the percentage of steatosis (FIG. 11).

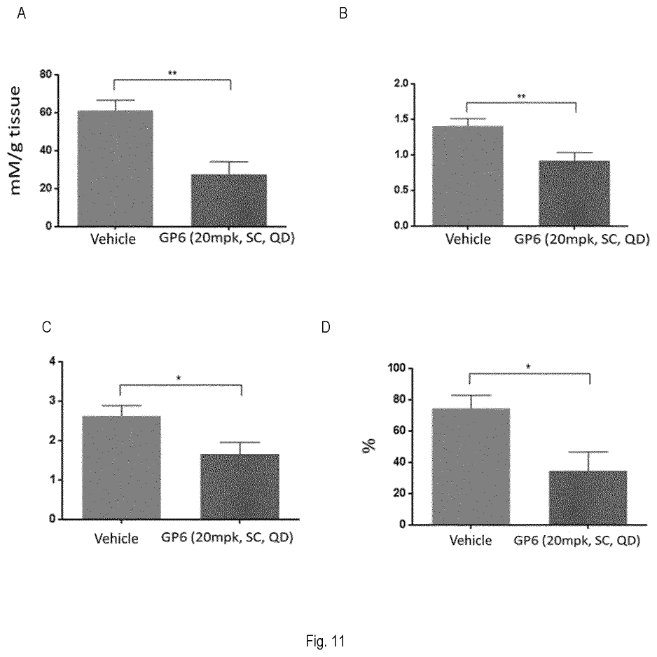

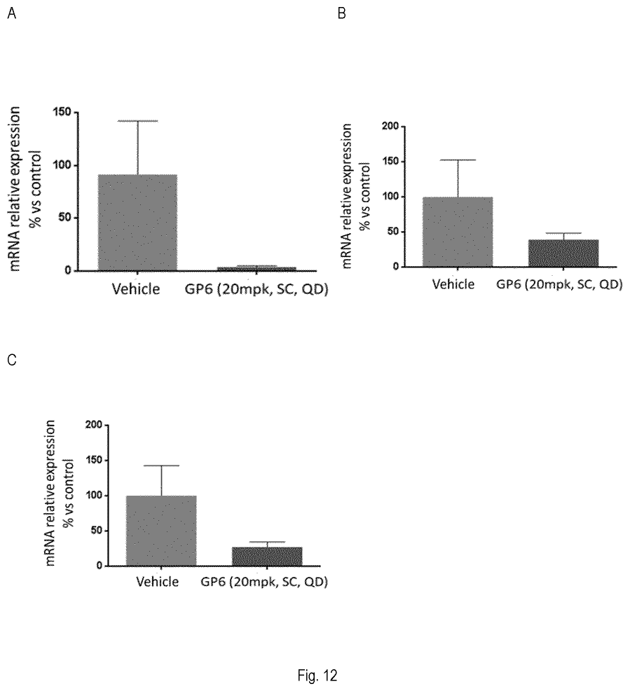

The reduction of the hepatic lipogenesis and reduced hepatic lipid accumulation was also corroborated by measuring PPARY mRNA levels in hepatic tissue, which is the major transcription factor regulating the expression of key hepatic lipogenic enzymes (ACC and FAS). As it can be seen below, daily subcutaneous treatment with GP6 (20 mg/kg/day) reduced the mRNA levels of PPARY, ACC and FAS when compared with vehicle treated mice (FIG. 12).

### GP6 Treatment Reduces Hepatic Inflammation

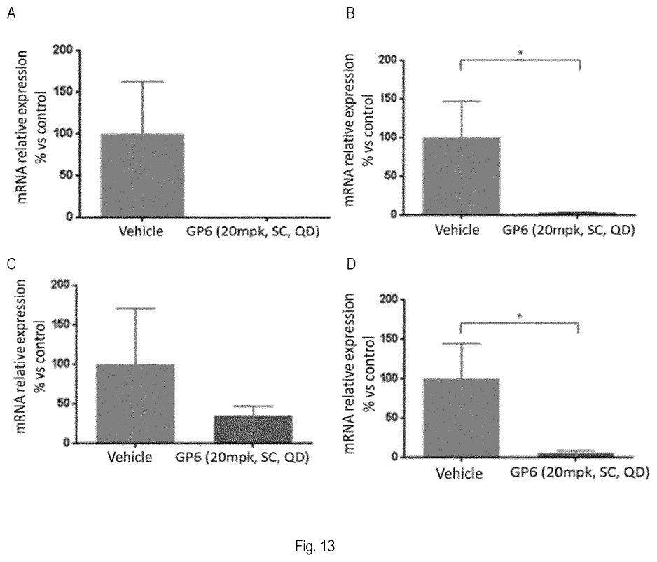

Daily subcutaneous injection with GP6 (20 mg/kg/day) reduced hepatic inflammation when compared with vehicle treated mice. This was evident since GP6 treatment reduced expression of F480 mRNA levels in the corresponding hepatic tissue a marker for macrophage infiltration. In addition, GP6 treatment reduced the hepatic expression of proinflammatory cytokines such as TNFα, IL1P and IL6 when compared with vehicle treated mice (FIG. 13).

### GP6 Treatment Reduces Adipose Tissue Inflammation

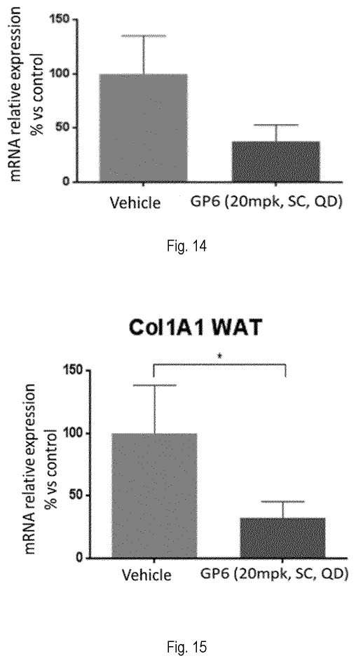

Daily subcutaneous injection with GP6 (20 mg/kg/day) reduced adipose tissue inflammation when compared with vehicle treated mice. This was evident since GP6 treatment reduced expression of F480 mRNA levels in adipose tissue (a marker for macrophage infiltration) when compared with vehicle treated mice (FIG. 14).

### GP6 Treatment Reduces Adipose Tissue Fibrosis

Daily subcutaneous injection with GP6 (20 mg/kg/day) reduced adipose tissue fibrosis when compared with vehicle treated mice. This was evident since GP6 treatment Col1A1 mRNA expression in adipose tissue when compared with vehicle treated mice (FIG. 15).

These assays in an obesity mouse model of obesity and NAFLD with peptide GP6 allow formulating the following conclusions:
GP6 protects against obesity development by reducing total body weight gain and subcutaneous and visceral adipose tissue weight induced by a high fat diet feeding and 30% fructose in the drinking water.
GP6 protects against fatty liver disease development by reducing hepatic total liver weight and lipid accumulation induced by a high fat diet feeding and 30% fructose in the drinking water. Mechanistically,
GP6 reduced the hepatic lipogenesis by inhibiting the expression PPARY mRNA levels, which is the major transcription factor regulating hepatic lipogenesis and by reducing the expression of key hepatic lipogenic enzymes such as ACC and FAS.
GP6 protects against hepatic and adipose tissue inflammation induced by a high fat diet feeding and 30% fructose in the drinking water by reducing the expression of F480 mRNA levels, a marker for macrophage infiltration in liver and adipose tissue as well as by reducing the hepatic expression of proinflammatory cytokines such as TNFα, IL1β and IL6.
GP6 protects against adipose tissue fibrosis development induced by a high fat diet feeding and 30% fructose in the drinking water by reducing the expression of Cola1a1 mRNA levels.

### Example 5. Preventive Effects of Peptide GP6 (SEQ ID NO. 16) in a CCl4-Induced Fibrosis Mouse Model

### Materials and Methods

### Animals

C57BL6/J male mice were purchased and kept for quarantine and acclimatization for a period of about 7 days before initiation of every experiment. Mice were housed in clean and sterilized mice cages and maintained in a controlled environment with 22+3° C. temperature, 50+20% humidity, a light/dark cycle of 12 hours each and 15-20 fresh air changes per hour.

### In Vivo Experiments

Preventive effect of a daily subcutaneous injection of GP6 (20 mg/kg/day) in a mouse model of fibrosis induced by twice a week intraperitoneal injection of CCl₄ (1 ml/Kg diluted in olive oil 1:5) during a 4 weeks period. A group of control mice (n=6) was treated with daily subcutaneous injections of vehicle (water) and the other group (n=10) with daily subcutaneous injection of GP6 (20 mg/kg/day) during 10 weeks.

At the end of the experiment mice were sacrificed, serum samples were extracted, tissues were weighted and part was fixed in paraformaldehyde for histology studies and part immediately frozen at −80 C for mRNA and protein expression studies.

All experimental procedures were approved by the Institutional Animal Use Subcommittees of Vall Hebron University Hospital Research Institute and the Autonomous University of Barcelona.

### Histology, AST and ALT Measurements, RNA Analysis

The Histology studies, AST and ALT measurements as well the RNA analysis were performed as described in example 4.

All the results are shown as Mean±SEM in the figures. One-way ANOVA and Bonferroni's post-hoc test compared to Control and/or Vehicle group were performed depending on the experiment. * p<0.05 and **p<0.01

### Results

### GP6 Treatment Partially Reduces Total Body Weight Lost Induced by CCl₄ Treatment

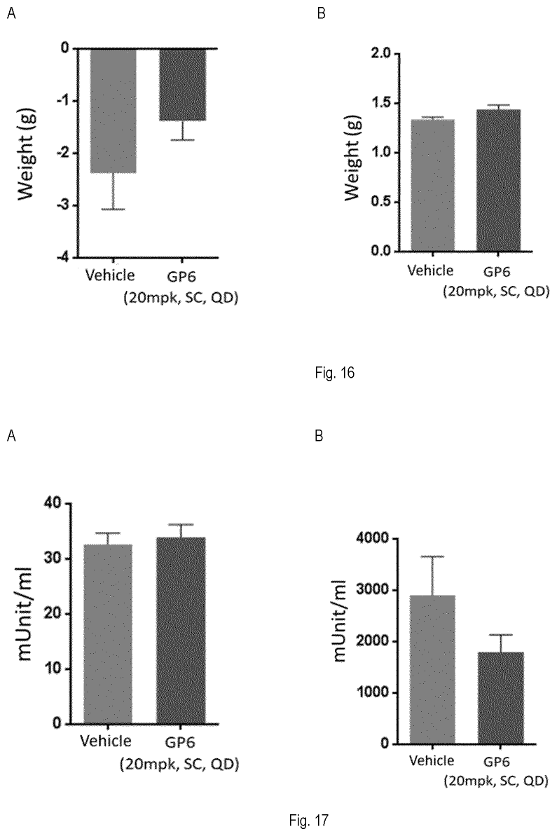

Total body weight was monitored once a week during the 4-week experiment. As shown below, mice treated with vehicle lost more body weight when compared with GP6 treated mice. Three mice in the vehicle group were sacrificed before the end of the experiment because their total weight lost exceeded 12.5% of the initial total body weight. Mice liver weight did not show any significance between groups (FIG. 16).

### GP6 Treatment Reduces ALT Serum Levels

Serum analysis showed that daily subcutaneous treatment with GP6 (20 mg/kg/day) reduced the ALT serum levels (approximately by 50%) but not the AST serum levels when compared with vehicle treated mice (FIG. 17).

### GP6 Treatment Reduces Hepatic Fibrosis

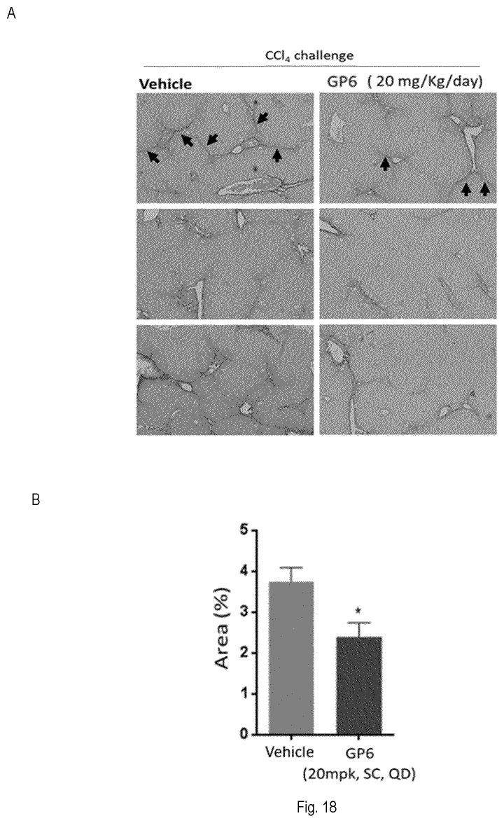

Histology studies using picrosirius staining (collagen staining) showed that CCl₄ treatment was effective in producing fibrosis in all mice. The pathologist confirmed that all mice showed stage 3 (F3) fibrosis. However, daily subcutaneous treatment with GP6 (20 mg/kg/day) reduced significantly hepatic fibrosis by approximately 37% when compared with vehicle treated mice as shown in the images and after quantification of all mice using ImageJ program (minimum of 3 pictures per section at 10×) (FIG. 18).

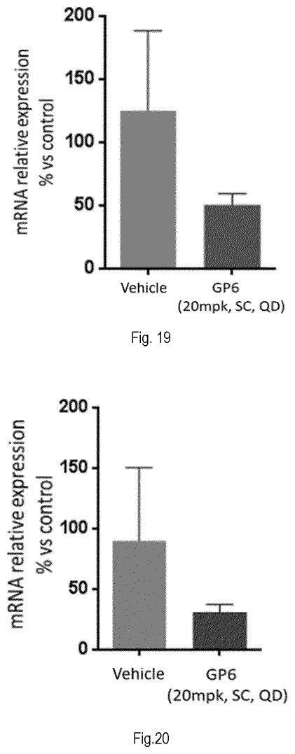

The reduction of fibrosis was also corroborated at expression level. Daily subcutaneous treatment with GP6 (20 mg/kg/day) reduced Collagen (Col1A1) expression in hepatic tissue by more than 50% when compared with vehicle treated mice (FIG. 19).

### GP6 Treatment Reduces Hepatic Inflammation

Daily subcutaneous treatment with GP6 (20 mg/kg/day) reduced significantly hepatic inflammation by more than 50% when compared with vehicle treated mice as shown by the reduction in F4/80 mRNA levels in the corresponding hepatic tissue, a well-known marker of macrophages infiltration during inflammation (FIG. 20).

These assays with peptide GP6 (SEQ ID NO. 16) in a CCl4-induced fibrosis mouse model allow formulating the following conclusions:
GP6 protects against hepatic fibrosis development induced by CCl₄ treatment by reducing hepatic collagen accumulation. Mechanistically, GP6 reduced the expression of hepatic Cola1a1 mRNA levels.
GP6 protects against hepatic inflammation induced by CCl₄ treatment by reducing the expression of F480 mRNA levels, a marker for macrophage infiltration in liver.

### Example 6. Therapeutic Effects of Peptide GP6 (SEQ ID NO. 16) in an Obesity and Fatty Liver Disease Mouse Model

### Materials and Methods

### Animals

C57BL6/J male mice were purchased and kept for quarantine and acclimatization for a period of about 7 days before initiation of every experiment. Mice were housed in clean and sterilized mice cages and maintained in a controlled environment with 22+3° C. temperature, 50+20% humidity, a light/dark cycle of 12 hours each and 15-20 fresh air changes per hour.

### In Vivo Experiments

For Obesity and NAFLD induction, the animals were fed ad libitum, either with chow diet (150SP-10, Safe Diets) or 45% high-fat diet (TD.190179, Envigo) plus 30% fructose (F0127, Sigma Aldrich) in drinking water for 20 weeks. The therapeutic effect of a daily subcutaneous injection of GP6 (20 mg/kg/day) in this mouse model was assessed for 10 weeks, starting the treatment on week 10 of the experiment until week 20.

A group of control mice (n=10) was fed with control diet and regular drinking water and a group of mice (n=30) obesity and fatty liver disease was induced for 10 weeks. At this point, mice were randomly divided in three groups of n=10, the first group was treated with daily subcutaneous injections of vehicle (water), the second group was treated with Telmisartan (5 mg/kg/day) in the drinking water and a subcutaneous injection of vehicle, and the third group with daily subcutaneous injection of GP6 (20 mg/kg/day) during 10 weeks.

At the end of the experiment mice were sacrificed, serum samples were extracted, tissues were weighted and part was fixed in paraformaldehyde for histology studies and part immediately frozen at −80 C for mRNA and protein expression studies as well as assessment of triglycerides.

All experimental procedures were approved by the Institutional Animal Use Subcommittees of Vall Hebron University Hospital Research Institute and the Autonomous University of Barcelona.

### Histology, AST and ALT Measurements, TG Measurements, RNA Analysis

The histology studies, AST and ALT measurements, TG measurements, and the RNA analysis were performed as described in Example 4.

All the results are shown as Mean±SEM in the figures. One-way ANOVA and Bonferroni's post-hoc test compared to Control and/or Vehicle group were performed depending on the experiment. * p<0.05 and **p<0.01.

### Results

### GP6 Treatment Reduces Total Body Weight and Liver

Total body weight was significantly increased by high fat diet and 30% fructose in the drinking water in the vehicle and Telmisartan (as positive control) treated group when compared with control mice, while GP6 treated mice were not significantly heavier than control mice at the end of the experiment.

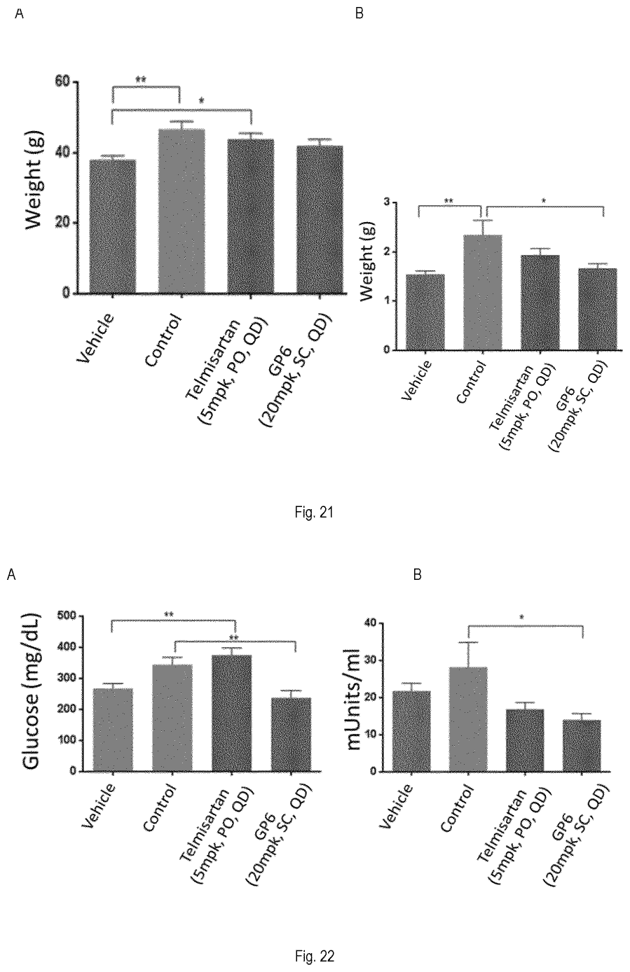

Results also showed liver weight was increased by high fat diet and 30% fructose in the drinking water in the vehicle treated mice when compared with control mice. While Telmisartan did not reduced significantly the liver weight, GP6 treatment reduced significantly when compared with vehicle treated mice (FIG. 21).

### GP6 Treatment Reduces Glucose and AST Levels

Serum analysis showed that daily subcutaneous treatment with GP6 (20 mg/kg/day) reduced the increase glucose levels induced by high fat diet and 30% fructose in the drinking water when compared with vehicle treated mice while Telmisartan did not have any effect when compared with vehicle treated mice. AST serum levels were also reduced significantly by the GP6 treatment (FIG. 22).

### GP6 Treatment Reduces Hepatic Lipid Accumulation

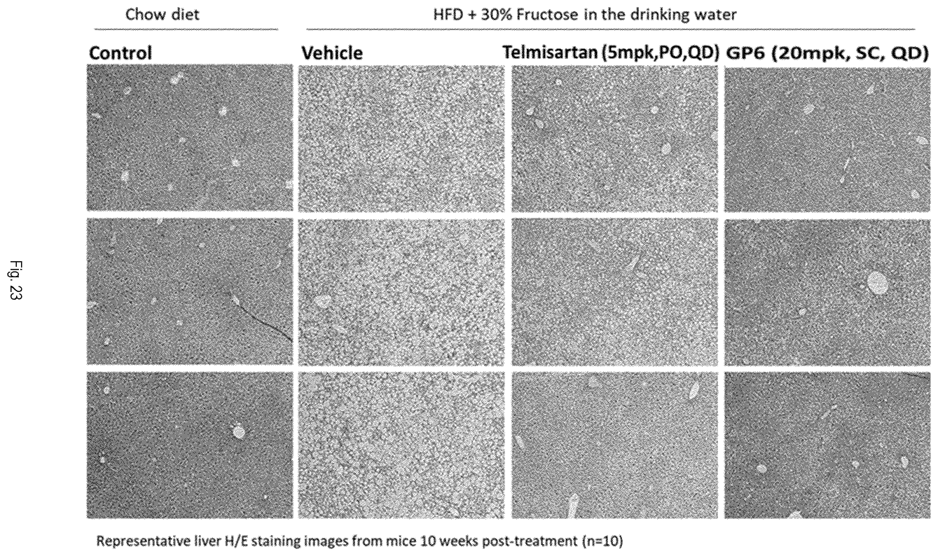

Histology studies showed that daily subcutaneous treatment with GP6 (20 mg/kg/day) reduced significantly hepatic lipid accumulation when compared with vehicle treated mice and to a greater extend than Telmisartan treatment (FIG. 23).

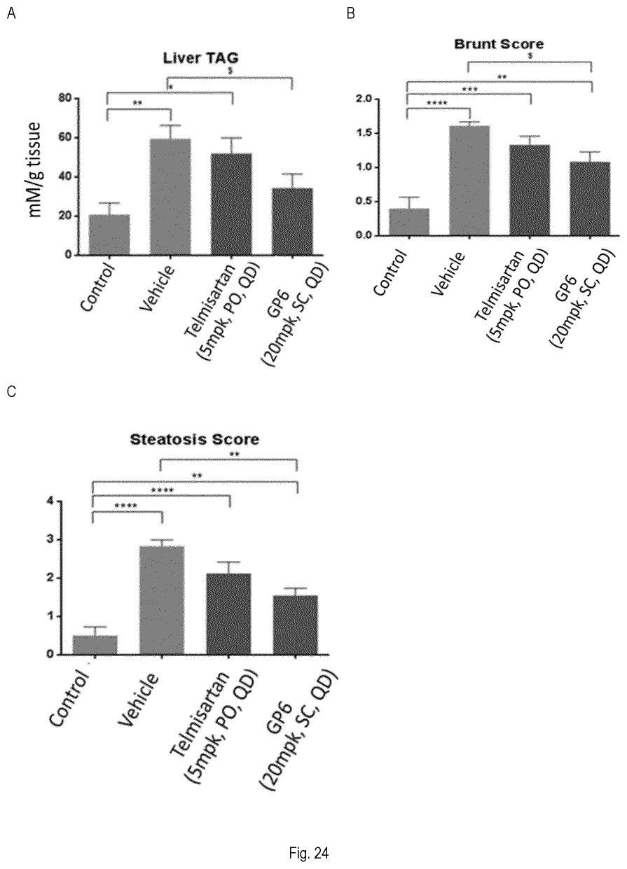

The results obtained in the above histological studies were confirmed by measuring hepatic triglyceride content of the mice (Liver TAG) and by the blind analysis of an expert liver pathologist (Dr. Maria Teresa Salcedo Allende) from the Pathology Department of the Vall d'Hebron Hospital assessing the Steatosis and Brunt Scores. As it can be seen below, GP6 treatment reduced significantly the lipid content increased induced by high fat diet and 30% fructose in the drinking water observed in the vehicle treated mice while Telmisartan treatment failed to decrease TAG liver content. These results were further corroborated by the pathologist using the Brunt and Steatosis scores (FIG. 24).

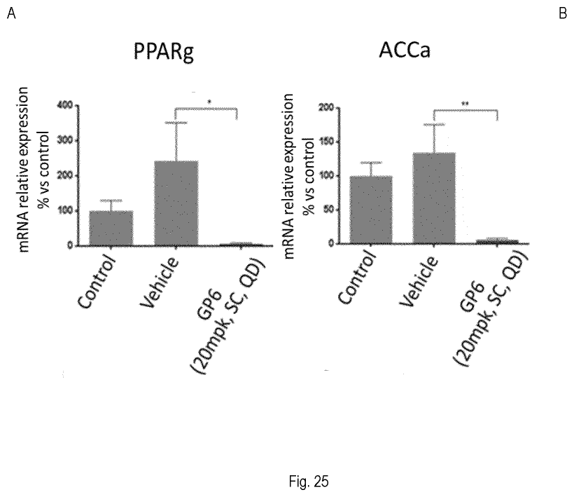

The antilipogenic activity of GP6 was also corroborated and supported by measuring PPARY mRNA levels, which is the major transcription factor regulating the expression of a key hepatic lipogenic enzymes such as ACC. As it can be seen below, daily subcutaneous treatment with GP6 (20 mg/kg/day) reduced the mRNA levels of PPARY and ACC in hepatic tissue when compared with vehicle treated mice (FIG. 25).

### GP6 Treatment Reduces Hepatic Fibrosis

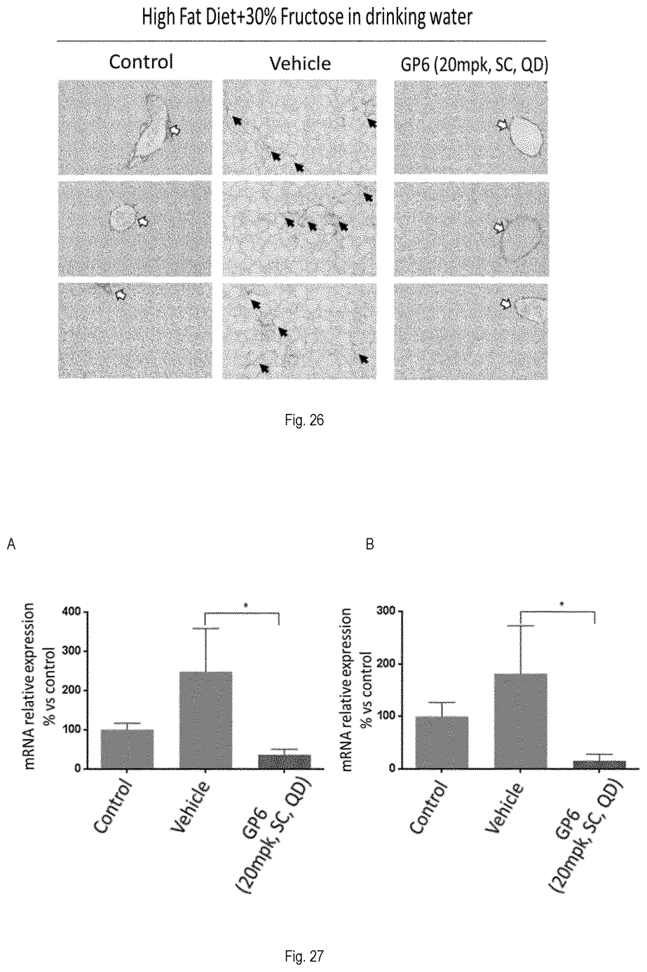

Mice treated with vehicle started developing hepatic fibrosis as seen in histological studies using picrosirius staining (collagen staining). Mice treated daily with subcutaneous injections of GP6 (20 mg/kg/day) did not showed hepatic fibrosis (FIG. 26).

The reduction in fibrosis appearance was further corroborated in liver samples assessing TGFβ1 and Col1A1 mRNA expression. TGFβ1 is the main factor activating hepatic stellate cells which will produce collagen in fibrosis development. Mice treated daily with subcutaneous injections of GP6 (20 mg/kg/day) showed reduced expression levels of TGFβ1 and Col1A1 in hepatic tissue when compared with vehicle treated mice(FIG. 27).

### GP6 Treatment Reduces Hepatic Inflammation

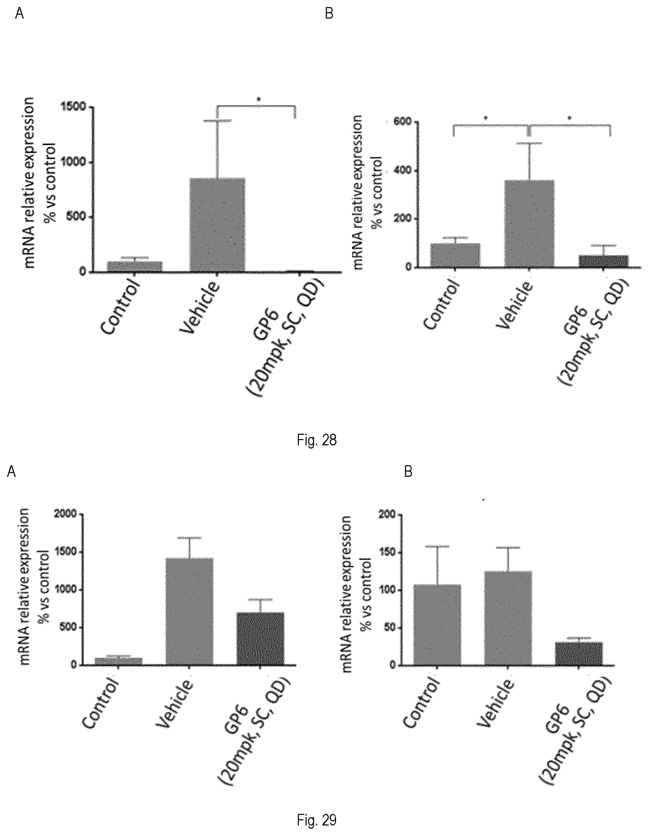

Daily subcutaneous injection with GP6 (20 mg/kg/day) reduced hepatic inflammation when compared with vehicle treated mice. This was evident since GP6 treatment reduced expression of F480 mRNA levels in hepatic tissue, a marker for macrophage infiltration. In addition, GP6 treatment reduced the hepatic expression of TNFα, a proinflammatory cytokine when compared with vehicle treated mice (FIG. 28).

### GP6 Treatment Reduces Adipose Tissue Inflammation

Daily subcutaneous injection with GP6 (20 mg/kg/day) reduced adipose tissue inflammation when compared with vehicle treated mice. This was evident since GP6 treatment reduced expression of F480 mRNA levels (a marker for macrophage infiltration) and IL1β (a proinflammatory cytokines) when compared with vehicle treated mice (FIG. 29).

### GP6 Treatment Reduces Adipose Tissue Fibrosis

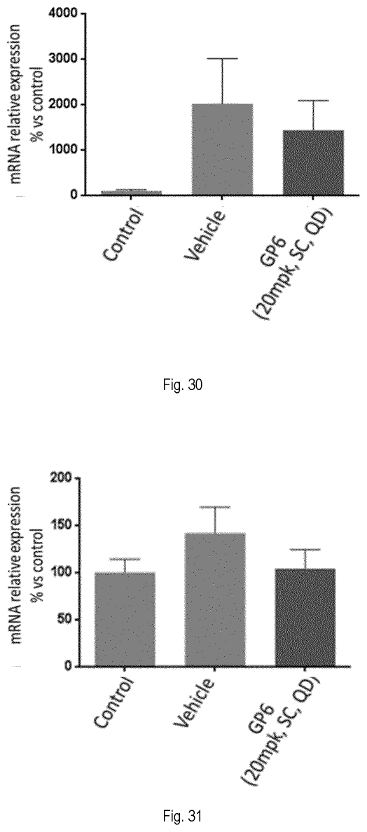

Daily subcutaneous injection with GP6 (20 mg/kg/day) reduced adipose tissue fibrosis when compared with vehicle treated mice. This was evident since GP6 treatment Col1A mRNA expression when compared with vehicle treated mice (FIG. 30).

GP6 treatment reduces heart fibrosis Daily subcutaneous injection with GP6 (20 mg/kg/day) reduced heart fibrosis when compared with vehicle treated mice. This was evident since GP6 treatment Col1A1 mRNA expression when compared with vehicle treated mice (FIG. 31).

These assays with peptide GP6 (SEQ ID NO. 16) in an obesity and fatty liver disease mouse model allow formulating the following conclusions:

GP6 protects against obesity development by reducing total body weight gain induced by a high fat diet feeding and 30% fructose in the drinking water.
GP6 protects against hyperglycemia induced by a high fat diet feeding and 30% fructose in the drinking water.
GP6 reduces fatty liver disease by reducing hepatic lipid accumulation induced by a high fat diet feeding and 30% fructose in the drinking water. Mechanistically, GP6 reduced the hepatic lipogenesis by inhibiting the expression PPARY mRNA levels, which is the major transcription factor regulating hepatic lipogenesis and by reducing the expression of key hepatic lipogenic enzyme such as ACC.
GP6 protects against hepatic fibrosis development induced by a high fat diet feeding and 30% fructose in the drinking water by reducing the expression of Cola1a1 and TGFβ-1 mRNA levels.
GP6 reduces hepatic and adipose tissue inflammation induced by a high fat diet feeding and 30% fructose in the drinking water by reducing the expression of F480 mRNA levels, a marker for macrophage infiltration in liver and adipose tissue as well as by reducing the hepatic expression of proinflammatory cytokines such as TNFα and IL1β.
GP6 protects against adipose tissue and heart fibrosis development induced by a high fat diet feeding and 30% fructose in the drinking water by reducing the expression of Cola1a1 mRNA levels.

Results as described in examples 2-6 were also obtained with the rest of peptides shown in table 5.

### Example 7

### Materials and Methods

### Peptide Synthesis

Peptides GP10 with SEQ ID NO.180: ATKTSASFEVRTWDPEAVIFY, GP11 with SEQ ID NO. 181: TKTASAFEVRTWDPEGVIFYGDTNPKDDWF, GP12 with SEQ ID NO. 182: TKTASAFEVRTWDPEGVIFY and GP13 with SEQ ID NO. 183: TKTSASFEVRTWDPEGVIFYADTNPKDDWF, where obtained following the synthesis route indicated above for peptide GP6.

### Cell Culture Experiments

Cell culture reagents were from Life Technologies Inc (Invitrogen SA, Barcelona, Spain). HepG2 hepatoblastoma cells (cat. no. HB-8065; ATCC, LGC Standards SLU, Barcelona, Spain) were maintained in Dulbecco's modified Eagle medium (DMEM) supplemented with 10% fetal bovine serum and antibiotics.

### HepG2 Cell Experiments

HepG2 cells were cultured for 5 day in the absence or presence of 30 mM fructose. Cells cultured in 30 mM fructose were daily treated with vehicle, Telmisartan, Semaglutide or EDL6D.
HepG2 cells were cultured for 24 h in the absence or presence of GP6 (40 μg/ml), GP10 (40 μg/ml), GP11 (40 μg/ml), GP12 (40 μg/ml), GP13 (40 μg/ml) or SHBG (20 nM).
HepG2 cells were cultured in the absence or presence of 30 mM fructose and treated for 2 h with vehicle or GP4 (40 μg/ml), GP6 (40 μg/ml), GP7 (40 μg/ml), GP8 (40 μg/ml), GP9 (40 μg/ml), GP10 (40 μg/ml), GP11 (40 μg/ml), GP12 (40 μg/ml), GP13 (40 μg/ml) or SHBG (20 nM).

### LX-2 Cell Experiments

LX-2 human hepatic stellate cells (SCC064) were maintained in DMEM supplemented with 2% fetal bovine serum and antibiotics. For experiments, LX-2 were cultured for 24 h in DMEM 0.2% FBS and treated for 24 h with vehicle or TGFβ1 (20 ng/ml) in the absence or presence of EDL6D (10 or 40 μg/ml). At the end of the experiment's cells were harvested for gene expression analysis.

### Preadipocyte Cell Experiments

Human preadipocytes and cell culture reagents were from purchased from Tebu-Bio (Tebu-bio, Barcelona, Spain) and differentiation protocol was done following the manufacturer's instructions. Human adipocytes were maintained in a humidified incubator with 5% CO2 at 37° C. (Shez-Lopez et al Journal Nutritional Biochemistry 2020).

### Lipolysis Assay

Lipolysis assays were performed using a Lipolysis Kit (Tebu-Bio) following the manufacturer's instructions (Shez-Lopez et al Journal Nutritional Biochemistry 2020).

### RNA Analysis

Total RNA was extracted from HepG2 and LX-2 cells using TRIzol reagent (Invitrogen SA). Reverse transcription was performed at 42° C. for 50 minutes using 3 mg of total RNA, 200 U of Superscript II, and an oligo-dT primer and reagents provided by Invitrogen. An aliquot of the reverse transcription product was amplified in a 25-mL reaction using SYBRGreen (Invitrogen SA) with appropriate oligonucleotide primer pairs corresponding to human SHBG, ACC, FAS, ACLY, PPARG, and 18S and mouse ACC, FAS, ACLY, PPARG, Col1A1 and 18S. Results were analyzed using the 7000 Sequence Detection System program (Applied Biosystems, Thermo Fisher Scientific, Barcelona, Spain) (Shez-Lopez et al Endocrinology 2017).

### Histology

For histological studies, livers and adipose tissue were fixed in 4% paraformaldehyde for 24 hours and embedded in paraffin. Serial 5-mm-thick sections were used for histological examination and stained with hematoxylin eosin (H&E) and Picrosirius red for histological examination following standard procedures. Briefly, sections were alcohol-xylol hydrated at room temperature, and subsequently, they were stained at room temperature in a solution of 0.1% Direct Red 80 (Sigma-Aldrich) in a saturated aqueous solution of picric acid (Sigma-Aldrich) for 1 h. Following that, sections were rinsed twice in 0.5% acetic acid or 5 min, alcohol-xylol dehydrated and covered with Eukitt mounting medium (Sigma-Aldrich). These sections were examined with an Olympus BX61 microscope. Six random fields at 4× magnification from each slice were quantified using ImageJ software to determine the liver collagen deposition as the red stained area in the image. The stage of liver steatosis and/or fibrosis was assessed blinded by an experienced pathologist according to the NASH-clinical research network scoring system (Kleiner et al., 2005). (Briansó-Llort et al Journal Cellular Physiology 2022).

### AST and ALT Measurements

Serum levels of AST and ALT were measured using MAK055 and MAK052 from Sigma-Aldrich respectively following the manufacture's instructions.

### TG Measurements

Hepatic TG content was measured from liver extracts using a Triglyceride Assay Kit (ab65336) following the manufacture's instructions.

### Results

### Effect of GP6 and Modified Peptides on Hepatic Lipogenesis in HepG2 Cells.

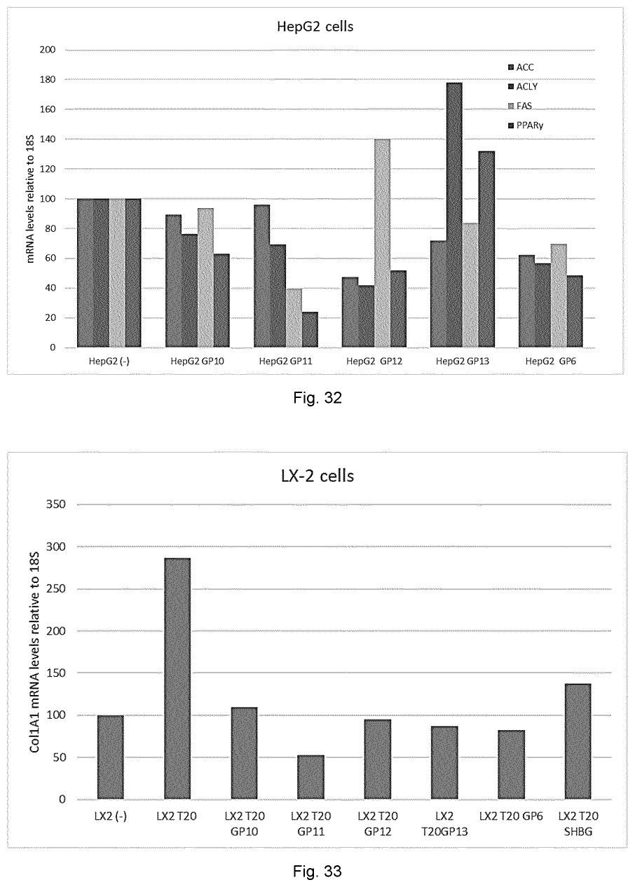

HepG2 cells were cultured for 24 h in the absence or presence of GP10, GP11, GP12, GP13 or GP6. Results showed that GP10, GP11 and GP6 reduced the mRNA levels of a key hepatic lipogenic enzyme such as Acetyl-CoA Carboxylase (ACC), ATP Citrate Lyase (ACLY) and Fatty Acid Synthase (FAS) and a key transcription factor regulating several lipogenic enzymes such as PPARγ when compared with vehicle treated HepG2 cells (FIG. 32). GP12 treatment reduced all key lipogenic genes except FAS and GP13 was able to reduce ACC and FAS mRNA levels but no ACLY and PPARγ.

### Effect of Different Peptides and SHBG on Fibrosis in LX-2 Cells

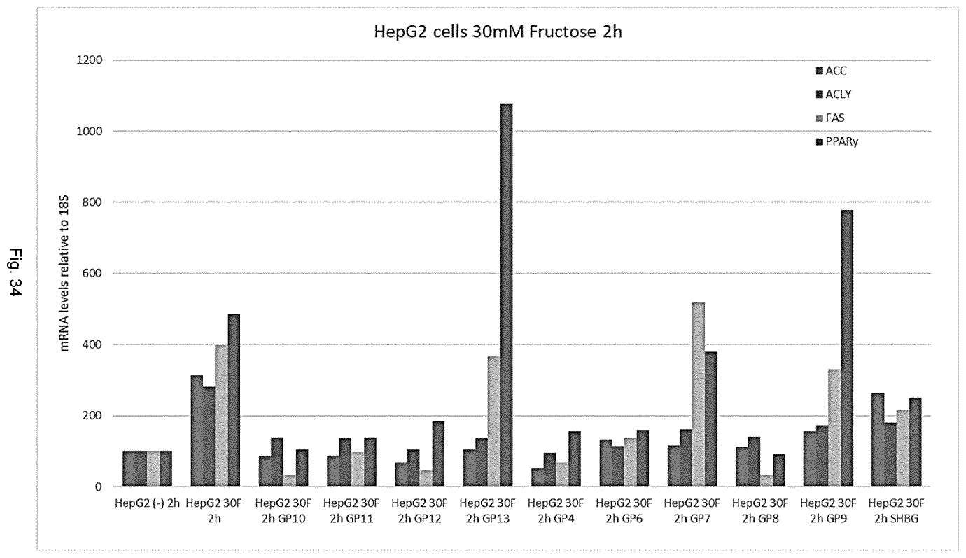

GP6 treatment reduced the mRNA levels of collagen 1A1 induced by TGFβ1 in human hepatic stellate cell line (LX-2). GP10, GP11, GP12 and GP13 carrying 2 aa substitutions had similar results than GP6 treatment. All peptides were more effective than SHBG in reducing Col1A1 expression (FIG. 33).

### Effects of Different Peptides and SHBG in Reducing Hepatic Lipogenic Key Enzymes in the Presence of 30 mM Fructose.

HepG2 cells were cultured in the absence or presence of 30 mM fructose for 2 h and treated with vehicle or GP10, GP11, GP12, GP13, GP4, GP6, GP7, GP8, GP9 or SHBG. Results showed that treatment with GP10, GP11, GP12, GP4, GP6, GP8 or SHBG was able to reduce the fructose-induced increase expression of key lipogenic enzymes (ACC, ACLY, FAS and PPARγ). GP13 and GP9 treatments were able to reduce the expression of ACC and ACLY more effectively than SHBG but had no effect on FAS and increased PPARγ expression. GP7 treatment was able to reduce the expression of ACC and ACLY more effectively than SHBG but had no effect on FAS and PPARγ expression (FIG. 34).

### CITATION LIST

EP 3003351 A1Altschul et al., “Basic local alignment search tool”, 1990, J. Mol. Biol, v. 215, pages 403-410.
Sex hormone-binding globulin overexpression protects against high-fat diet-induced obesity in transgenic male mice. Saez-Lopez C, Villena J A, Simo R, Selva D M. J Nutr Biochem. 2020 November; 85:108480; doi: 10.1016/j.jnutbio.2020.108480 Sex Hormone-Binding Globulin Reduction in Metabolic Disorders May Play a Role in NAFLD Development.Saez-Lopez C, Barbosa-Desongles A, Hernandez C, Dyer R A, Innis S M, Simo R, Selva D M. Endocrinology. 2017 Mar. 1; 158(3):545-559. doi: 10.1210/en.2016-1668.
Design and validation of a histological scoring system for nonalcoholic fatty liver disease. Kleiner D E, Brunt E M, Van Natta M, Behling C, Contos M J, Cummings O W, Ferrell L D, Liu Y C, Torbenson M S, Unalp-Arida A, Yeh M, McCullough A J, Sanyal AJ; Nonalcoholic Steatohepatitis Clinical Research Network. Hepatology. 2005 June; 41(6):1313-21. doi: 10.1002/hep.20701.
Transforming growth factor-beta 1: A new factor reducing hepatic SHBG production in liver fibrosis. Briansó-Llort L, Fuertes-Rioja L, Ramos-Perez L, Salcedo-Allende M T, Hernandez C, Simo R, Selva D M. J Cell Physiol. 2022 September; 237(9):3598-3613. doi: 10.1002/jcp.30818.

What is claimed is:

1. A peptide, or a pharmaceutically acceptable salt thereof, wherein the peptide has a length of 10 to 40 amino acids, each one of the amino acids being a L- or D-amino acid, and consists of a peptide fragment F1 and, additionally: of a peptide fragment F2 at the N-terminal end of peptide fragment F1 and/or of a peptide fragment F3 at the C-terminal end of peptide fragment F1, wherein,
i) peptide fragment F1 has SEQ ID NO. 9: TWDPEGVIFY or is a sequence variant thereof that has at least 80% sequence identity with SEQ ID NO.9, wherein each one of the amino acids of peptide fragment F1 is a L- or D-amino acid,
ii) peptide fragment F2 has SEQ ID NO. 13 (TKTSSSFEVR) or is a sequence variant thereof with at least 80% sequence identity with SEQ ID NO.13; or has SEQ ID NO. 14 (VMTFDLTKITKTSSSFEVR) or is a sequence variant thereof with at least 80% sequence identity with SEQ ID NO.14; and wherein each one of the amino acids of peptide fragment F2 is a L- or D-amino acid;
and
iii) peptide fragment F3 has SEQ ID NO. 4 (GDTNPKDDWF) or is a sequence variant thereof with at least 80% sequence identity with SEQ ID NO.4; or has SEQ ID NO. 15 (GDTNPKDDWFMLGLRDGRPE) or is a sequence variant thereof with at least 80% sequence identity with SEQ ID NO.15; and wherein each one of the amino acids of peptide fragment F3 is a L- or D-amino acid.

2. The peptide, or pharmaceutically acceptable salt thereof, according to claim 1 wherein:
the peptide fragment F1 that is a sequence variant of SEQ ID NO. 9 substantially maintains or improves the activity of peptide fragment F1 with SEQ ID NO. 9 in the peptide,
the peptide with F1 being a sequence variant of SEQ ID NO. 9 substantially maintains or improves the activity of the same peptide comprising instead of F1 as sequence variant of SEQ ID NO.9, F1 with SEQ ID NO.9, or
the combination thereof.

3. (canceled)

4. The peptide or pharmaceutically acceptable salt thereof, according to claim claim 1, wherein:
the peptide fragment F2 that is a sequence variant of SEQ ID NO.13 substantially maintains or improves the activity of peptide fragment F2 with SEQ ID NO. 13 in the peptide,
the peptide with F2 being a sequence variant of SEQ ID NO. 13 substantially maintains or improves the activity of the same peptide comprising instead of F2 as sequence variant of SEQ ID NO.13, F2 with SEQ ID NO.13, or
the combination thereof.

5. (canceled)

6. The peptide or pharmaceutically acceptable salt thereof, according to claim 1, wherein:
the peptide fragment F2 that is a sequence variant of SEQ ID NO.14, substantially maintains or improves the activity of peptide fragment F2 with SEQ ID NO. 14 in the peptide,
the peptide with F2 being a sequence variant of SEQ ID NO. 14 substantially maintains or improves the activity of the same peptide comprising instead of F2 as sequence variant of SEQ ID NO.14, F2 with SEQ ID NO.14, or
the combination thereof.

7. (canceled)

8. The peptide or pharmaceutically acceptable salt thereof, according to claim claim 1, wherein:
the peptide fragment F3 that is a sequence variant of SEQ ID NO.4 substantially maintains or improves the activity of peptide fragment F3 with SEQ ID NO. 4 in the peptide,
the peptide with F3 being a sequence variant of SEQ ID NO. 4 substantially maintains or improves the activity of the same peptide comprising instead of F3 as sequence variant of SEQ ID NO.4, F3 with SEQ ID NO.4, or
the combination thereof.

9. (canceled)

10. The peptide or pharmaceutically acceptable salt thereof, according to claim claim 1, wherein:
the peptide fragment F3 that is a sequence variant of SEQ ID NO.15 substantially maintains or improves the activity of peptide fragment F3 with SEQ ID NO. 15 in the peptide,
the peptide with F3 being a sequence variant of SEQ ID NO. 15 substantially maintains or improves the activity of the same peptide comprising instead of F3 as sequence variant of SEQ ID NO.15, F3 with SEQ ID NO.13,
or the combination thereof.

11. (canceled)

12. The peptide, or pharmaceutically acceptable salt thereof, according to claim 1 wherein the peptide has a sequence selected from the list consisting of:
SEQ ID NO. 16 (TKTSSSFEVRTWDPEGVIFY), or a sequence variant thereof at least 90% identical to SEQ ID NO.16, that substantially maintains or improves the activity of SEQ ID NO.16,
SEQ ID NO. 17 (TWDPEGVIFYGDTNPKDDWF), or a sequence variant thereof at least 90% identical to SEQ ID NO.17 that substantially maintains or improves the activity of SEQ ID NO.17,
SEQ ID NO. 18 (TKTSSSFEVRTWDPEGVIFYGDTNPKDDWF), or a sequence variant thereof at least 90% identical to SEQ ID NO.18 that substantially maintains or improves the activity of SEQ ID NO.18,
SEQ ID NO. 19 (TWDPEGVIFYGDTNPKDDWFMLGLRDGRPE), or a sequence variant thereof at least 90% identical to SEQ ID NO.19 that substantially maintains or improves the activity of SEQ ID NO.19, and
SEQ ID NO. 20 (VMTFDLTKITKTSSSFEVRTWDPEGVIFY), or a sequence variant thereof at least 90% identical to SEQ ID NO.20 that substantially maintains or improves the activity of SEQ ID NO. 20.

13.-16. (canceled)

17. The peptide or pharmaceutically acceptable salt thereof, according to claim 1, wherein:
F1 has SEQ ID NO.9,
F2 has SEQ ID NO. 13 or SEQ ID NO.14, and
F3 has SEQ ID NO. 4 or SEQ ID NO.15.

18.-22. (canceled)

23. The peptide, or pharmaceutically acceptable salt thereof, according to claim 1 wherein the peptide has a sequence selected from the group consisting of:
SEQ ID NO. 180
(ATKTSASFEVRTWDPEAVIFY).
SEQ ID NO. 181
(TKTASAFEVRTWDPEGVIFYGDTNPKDDWF),
SEQ ID NO. 182
(TKTASAFEVRTWDPEGVIFY),
and
SEQ ID NO. 183
(TKTSASFEVRTWDPEGVIFYADTNPKDDWF).

24.-26. (canceled)

27. The peptide, or pharmaceutically acceptable salt thereof according to claim 1, wherein:
(i) at least one amino acid of the peptide is a D-amino acid, preferably all the amino acids of the peptide are D-amino acids, and/or
(ii) the N-terminal end of the peptide corresponds to —NR₁R₂, being R₁ and R₂ independently selected from the group consisting of —H, —C(O)R₃, a triglyceride, (C₁-C₂₀)alkyl and (C₁-C₂₀)alkenyl and R₃ is —H, (C₁-C_{2M})alkyl, or (C₁-C₂₀)alkenyl; and
(iii) the C-terminal end of the peptide corresponds to —COOR₄ or —C(O)NR₅R₆ wherein R₄ to R₆ are radicals independently selected from the group consisting of: —H and (C₁-C_{2M})alkyl.

28. A pharmaceutical composition comprising a therapeutical effective amount of a peptide, or pharmaceutically acceptable salt thereof, as defined in claim 1 and one or more therapeutically acceptable carriers.

29. (canceled)

30. A method for treating a condition or disease caused or associated to the accumulation of fat in an organ or tissue, inflammation and/or fibrosis the method comprising administering a therapeutically effective amount of a peptide as defined in claim 1 to a subject in need thereof.

31. The method according to claim 30, wherein the disease is selected from the group consisting of non-alcoholic steatohepatitis (NASH), hepatic fibrosis, cirrhosis, hepatic steatosis, overweight, obesity, inflammatory disease, lipohyperdystrophy, diabetes, and cardiovascular disease.

32. The method according to claim 31, wherein the disease is non-alcoholic steatohepatitis (NASH).

33. A cosmetic composition comprising the peptide, or pharmaceutically acceptable salt thereof, according to claim 1 and one or more cosmetically acceptable carriers.

34. (canceled)

35. A cosmetic method for reducing the subcutaneous fat in a mammal, preferably accumulated within the fibrous connective tissue under the skin of the mammal, such as cellulite, the method comprising the topical, dermal or oral administration of the peptide as defined in claim 1.

36. (canceled)

37. A kit of parts comprising:
a peptide or pharmaceutically acceptable salt thereof as defined in claim 1, and
a device selected from the group consisting of an injection device, an implant, or a biodegradable container.

38. A device selected from the group consisting of an injection device, an implant, and a biodegradable container, the device comprising the peptide as defined in claim 1.

39. A method for treating a condition or disease caused or associated to the accumulation of fat in an organ or tissue, inflammation and/or fibrosis, the method comprising administering a therapeutically effective amount of a pharmaceutical composition as defined in claim 11 to a subject in need thereof.

40. A method for treating a condition or disease selected from the group consisting of non-alcoholic steatohepatitis (NASH), hepatic fibrosis, cirrhosis, hepatic steatosis, overweight, obesity, inflammatory disease, lipohyperdystrophy, diabetes, and cardiovascular disease, the method comprising administering a therapeutically effective amount of a pharmaceutical composition as defined in claim 28 to a subject in need thereof.

41. A kit of parts comprising:
a pharmaceutical composition as defined in claim 28,
a device selected from the group consisting of an injection device, an implant, or a biodegradable container.
